SEQUENCE LISTING (27 sequences)

SEQ ID NO: 1 - protein, 8 aa, synthetic construct
GFPFSDYY

SEQ ID NO: 2 - protein, 8 aa, synthetic construct
INGDGGTT

SEQ ID NO: 3 - protein, 14 aa, synthetic construct
VRGRRITGTGAIDY

SEQ ID NO: 4 - protein, 121 aa, synthetic construct
DVKLVESGGGLVKVGGSLKISCAASGFPFSDYYMAWVGQTPEKRLELVAAINGDGGTTYYPDTVKGRFIISRDNAKNTLYLQMSSLRSEDTALYYCVRGRRITGTGAIDYWGQGTSVTVSS

SEQ ID NO: 5 - DNA, 363 bp, synthetic construct
  source 1..363: mol_type = other DNA
gacgtgaagctcgtggagtctgggggaggcttagtgaaggttggagggtccctaaaaatttcatgtgcagcctctggattccccttcagtgactattatatggcttgggttggccagactccagagaagaggctggagttggtcgcagccattaatggtgatggtggtaccacctactatccagacactgtgaagggccgattcatcatttccagagacaatgccaagaacaccctgtacctgcaaatgagcagtctgaggtctgaggatacagccttgtattattgtgtaagagggagaaggataactggaactggggctatagactactggggtcaaggaacctcagtcaccgtctcctca

SEQ ID NO: 6 - protein, 5 aa, synthetic construct
SNISY

SEQ ID NO: 7 - protein, 9 aa, synthetic construct
QIYHSYPYT

SEQ ID NO: 8 - protein, 106 aa, synthetic construct
QIFLTQSPVILSASPGEKVTMTCSATSNISYMYRYQQKPGSSPKPWIYGTSNLASRVPARFSGSGSGTSYSLTISNMEAEDAATYYCQIYHSYPYTFGGGTKLEIK

SEQ ID NO: 9 - DNA, 318 bp, synthetic construct
  source 1..318: mol_type = other DNA
caaatttttctcacccagtctccagttatcctgtctgcatctccaggggagaaggtcaccatgacctgcagtgccacctcaaatataagttacatgtacaggtaccagcagaagccaggatcctcacccaaaccctggatttatggcacatccaacctggcttctcgagtccctgctcgcttcagtggcagtggatctgggacctcttattctctcacaatcagcaacatggaggctgaagatgctgccacttattactgccagatatatcatagttacccgtacacgttcggaggggggaccaagctggaaataaaa

SEQ ID NO: 10 - protein, 8 aa, synthetic construct
GYSFTDYN

SEQ ID NO: 11 - protein, 8 aa, synthetic construct
IDPYNGGT

SEQ ID NO: 12 - protein, 13 aa, synthetic construct
AKGGYRYDAWFAY

SEQ ID NO: 13 - protein, 120 aa, synthetic construct
EIQLQQSGPELVKPGASVKVSCKASGYSFTDYNMYWVKQSHGKSLEWVGYIDPYNGGTTYNQKFKGKATLTVDKSSSTAFMHLNSLTSEDSAVYYCAKGGYRYDAWFAYWGQGTLVTVSA

SEQ ID NO: 14 - DNA, 360 bp, synthetic construct
  source 1..360: mol_type = other DNA
gagatccagctgcagcagtctggacctgagctggtgaagcctggggcttcagtgaaggtatcctgcaaggcttctggttactcattcactgactacaacatgtactgggtgaagcagagccatggaaagagccttgagtgggttggatatattgatccttacaatggtggtactacctacaaccagaagttcaagggcaaggccacattgactgttgacaagtcctccagcacagccttcatgcatctcaacagcctgacatctgaggactctgcagtctattactgtgcaaagggcggctataggtacgacgcctggtttgcttactggggccaagggactctggtcactgtctctgca

SEQ ID NO: 15 - protein, 11 aa, synthetic construct
QSLENNNGNTY

SEQ ID NO: 16 - protein, 9 aa, synthetic construct
LQVTHVPWT

SEQ ID NO: 17 - protein, 112 aa, synthetic construct
DAVMTQTPLSLPVSLGDHASISCRSSQSLENNNGNTYLNWYLQKPGQSPQLLIYRVSNRFSGVLDRFSGSGSGTDFTLKISRVEAEDLGVYFCLQVTHVPWTFGGGTKLEIK

SEQ ID NO: 18 - DNA, 336 bp, synthetic construct
  source 1..336: mol_type = other DNA
gatgctgtgatgacccaaactccactctccctgcctgtcagtcttggagatcacgcctccatctcttgcaggtctagtcagagccttgaaaacaataatggaaacacctatttgaactggtacctccagaaaccaggccagtctccacagctcctgatctacagggtttccaaccgattttctggggtcctagacaggttcagtggtagtggatcagggacagatttcacactgaaaatcagcagagtggaggctgaggatttgggagtttatttctgcctccaagttacacatgtcccgtggacgttcggtggaggcaccaagctggaaatcaaa

SEQ ID NO: 19 - protein, 8 aa, synthetic construct
GFTFTDYY

SEQ ID NO: 20 - protein, 10 aa, synthetic construct
IRNKANGYTT

SEQ ID NO: 21 - protein, 13 aa, synthetic construct
TRAIYYGYDGFAY

SEQ ID NO: 22 - protein, 122 aa, synthetic construct
EVKLVESGGGLVQPGGSLRLSCATSGFTFTDYYMSWVRQPPGKALEWLGFIRNKANGYTTEYSASVKGRFTISRDNSQSILYLQMNTLRAEDSATYYCTRAIYYGYDGFAYWGQGTLVTVSA

SEQ ID NO: 23 - DNA, 366 bp, synthetic construct
  source 1..366: mol_type = other DNA
gaggtgaagctggtggagtctggaggaggcttggtacagcctgggggttctctgagactctcctgtgcaacttctgggttcaccttcactgattactacatgagctgggtccgccagcctccaggaaaggcacttgagtggttgggttttattagaaacaaagctaatggttacacaacagagtacagtgcatctgtgaagggtcggttcaccatctccagagataattcccaaagcatcctctatcttcaaatgaacaccctgagagctgaggacagtgccacttattactgtacaagggccatctactatggttacgacgggtttgcttactggggccaagggactctggtcactgtctctgca

SEQ ID NO: 24 - protein, 11 aa, synthetic construct
QSLENSNGNTY

SEQ ID NO: 25 - protein, 9 aa, synthetic construct
LQVTHVPFT

SEQ ID NO: 26 - protein, 112 aa, synthetic construct
DAVMTQTPLSLPVSLGDQASISCRSSQSLENSNGNTYLNWYLQKPGQSPQLLIYRVSNRFSGVLDRFSGSGSGTDFTLKISRVEAEDLGVYFCLQVTHVPFTFGSGTKLEIK

SEQ ID NO: 27 - DNA, 336 bp, synthetic construct
  source 1..336: mol_type = other DNA
gatgctgtgatgacccaaactccactctccctgcctgtcagtcttggagatcaagcctccatctcttgcaggtctagtcagagccttgaaaacagtaatggaaacacctatttgaactggtacctccagaaaccaggccagtctccacagctcctgatctacagggtttccaaccgattttctggggtcctagacaggttcagtggtagtggatcagggacagatttcacactgaaaatcagcagagtggaggctgaggatttgggagtttatttctgcctccaagttacacatgtcccattcacgttcggctcggggacaaagttggaaataaaa

(19) United States
(12) Patent Application Publication
(10) Pub. No.: US 2025/0161428 A1
(43) Pub. Date: May 22, 2025

(54) PREVENTING/TREATING PSEUDOMONAS AERUGINOSA INFECTION

(71) Applicant: NATIONAL RESEARCH COUNCIL OF CANADA, Ottawa (CA)

(72) Inventors: Andrew David COX, Ottawa (CA); Evguenii VINOGRADOV, Ottawa (CA); Janelle SAUVAGEAU, Ottawa (CA)

(21) Appl. No.: 18/841,818

(22) PCT Filed: Mar. 7, 2023
(86) PCT No.: PCT/CA2023/050297

§ 371 Date: Aug. 27, 2024

Related U.S. Application Data
(60) Provisional application No. 63/317,245, filed on Mar. 7, 2022.

(51) Int. Cl. A61K 39/104; A61K 39/00; A61K 39/385; A61P 31/04; C07H 3/06; C07K 16/12; C07K 16/44; G01N 33/569

(52) U.S. Cl. CPC A61K 39/104; A61K 39/385; A61P 31/04; C07H 3/06; C07K 16/1214; C07K 16/44; G01N 33/56911; A61K 2039/6037; C07K 2317/24; C07K 2317/565; G01N 2333/21

(57) ABSTRACT

The present disclosure relates generally to compounds, compositions, antibodies, and methods for raising and utilising an immune response in a subject, comprising administering to a subject an isolated or synthesised Pseudomonas aeruginosa glycan, optionally linked to a carrier protein in the form of a glycoconjugate. Further provided is an antibody that selectively binds a Pseudomonas aeruginosa glycan or glycoconjugate as described in this disclosure.

### FIELD OF THE INVENTION

The present disclosure relates to the identification and synthesis of novel methylated rhamnose containing glycans, protein-glycan conjugates and their use as antigens and vaccines. Also disclosed are antibodies that selectively bind said glycans and glycoconjugates. Uses of these glycans, glycoconjugates, corresponding compositions, and antibodies in the treatment and prevention of Pseudomonas aeruginosa infections are discussed.

### BACKGROUND

In recent years, large pharmaceutical companies have moved away from antibiotic research due in part to the high risks and costs related to development compared to the potential rewards [1]. An alternative to new antibiotics is the development of vaccines to prevent infections caused by antibiotic resistant bacteria [2]. A vaccine against antibiotic resistant P. aeruginosa has yet to reach the market, even though some vaccines are in clinical trials [3-5].

P. aeruginosa has on its surface a broad range of glycans. As a result, glycoconjugate vaccines have been explored [5-7], though none of these vaccines have been approved for human use [5].

In addition to vaccine approaches, the therapeutic potential of antibodies to surface carbohydrate targets is also a consideration. Current examples of this approach include targeting Psl by Medimmune and targeting LPS O-antigen by Aridis [8-11]. The bi-specific antibody approach of Medimmune was not pursued beyond their phase 1 trail [12]; however the potential of a DNA encoded mAb therapy (DMab) showed promise and may alleviate the high cost of direct mAb therapeutics [13]. These studies suggest that antibodies against P. aeruginosa may have therapeutic applications.

### SUMMARY OF THE INVENTION

The present disclosure relates generally to compounds and compositions and vaccines comprising a novel isolated or synthesised Pseudomonas aeruginosa glycan antigen, optionally linked to a carrier protein in the form of a glycoconjugate. Further provided is an antibody that selectively binds a Pseudomonas aeruginosa glycan or glycoconjugate. Also disclosed are uses and methods of treatment, as well as methods for raising and utilising an immune response in a subject.

In one aspect, the present invention provides an antigenic compound comprising the oligosaccharide moiety of Formula A:

α-Rha3OMe(-4α-Rha3OMe)ₙ- Formula A

wherein n is 1-5, preferably 2-4, and wherein the 2-position in each Rha3OMe saccharide moiety is independently substituted with —OAc or —OH.

In one aspect, the present invention provides an antigenic compound comprising the oligosaccharide moiety of Formula A1:

α-Rha3OMe(-4α-Rha3OMe)ₙ-X Formula A1

wherein n is 1-5 (preferably 2-4), and X is —H or -(4α-Man3OMe)ₘ-handle; and m is 0, 1, or 2, preferably 0 or 1; wherein the 2-position in each Rha3OMe saccharide moiety is independently substituted with —OAc or —OH.

In one aspect, the present invention provides an antigenic compound, wherein the handle is —(CH2)_{z}NH₂ or 2-glyceraldehyde, where z is an integer selected from the group consisting of 1-5. In one aspect, the handle is —(CH2)₂NH₂, —(CH2)₃NH₂, or —(CH2)₃NH₂, when m is 0.

In one aspect, if m is 1 or 2, the handle is 2-glyceraldehyde.

In one aspect, the present invention provides an antigenic compound, selected from the group consisting of:
α-D-Rha3OMe-4-(α-D-Rha3OMe-4)₄-4-α-D-Man3OMe-2-glyceraldehyde-1d (OS2);
3-O-methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranose (pentasaccharide);
3-O-methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranose (tetrasaccharide)
3-O-methyl-α-D-rhamnopyranoside-(1→4)-3-O-methyl-α-D-rhamnopyranoside-(1→4)-3-O-methyl-α-D-rhamnopyranose (trisaccharide)

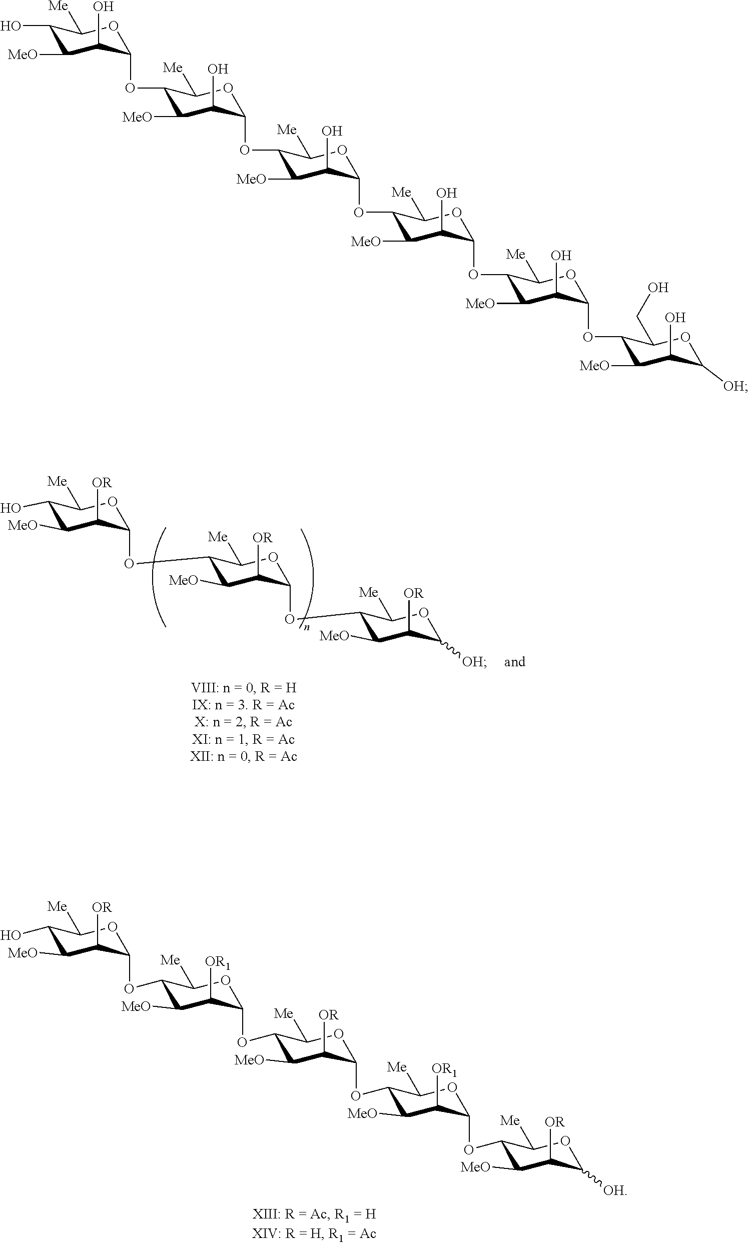

In one aspect, the present invention provides and antigenic compound comprising a linker for linkage to a carrier protein, and having Formula A2.

α-Rha3OMe(-4α-Rha3OMe)ₙ-X-Linker Formula A2

wherein n is 1-5, and X is -(4α-Man3OMe)ₘ-(handle)ₚ-; wherein m is 0, 1, or 2, preferably 0 or 1; and p is 0 or 1; and wherein the 2-position in each Rha3OMe saccharide moiety is independently substituted with —OAc or —OH

In one aspect, the present invention provides an antigenic compound comprising a conjugate of the antigenic compound conjugated to a carrier protein.

In one aspect, said conjugate has the following formula

α-Rha3OMe(-4α-Rha3OMe)ₙ-X-(Linker)_{q}-Carrier Protein Formula A3

wherein n is 1-5 (preferably 2-4), and X is -(4α-Man3OMe)ₘ-(handle)ₚ-; wherein m is 0, 1, or 2, preferably 0 or 1; and p is 0 or 1; and q is 0 or 1; and wherein the 2-position in each Rha3OMe saccharide moiety is independently substituted with —OAc or —OH.

In one aspect, the present invention the carrier protein comprises CRM197, tetanus toxoid (TT), a Pseudomonas aeruginosa protein, human serum albumin (HSA), bovine serum albumin (BSA), diphtheria toxin fragment B (DTFB), DTFB C8, Diphtheria toxoid (DT), fragment C of TT, pertussis toxoid, cholera toxoid, E. coli LT, E. coli ST, or exotoxin A from Pseudomonas aeruginosa. In one aspect, the carrier protein is CRM197, TT, or a Pseudomonas aeruginosa protein.

In one aspect, the present invention provides a pharmaceutical composition comprising the compound or the conjugate described herein; and a pharmaceutically acceptable diluent, carrier, or excipient. In one aspect, said pharmaceutical composition is a vaccine.

In one aspect, the present invention provides a method of raising an immune response in a subject, comprising administering to the subject: the compound, the conjugate, or the pharmaceutical composition described herein.

In one aspect, the present invention provides a method of preventing a P. aeruginosa infection in a subject, the method comprising administering to the subject: the compound, the conjugate, or the pharmaceutical composition described herein.

In one aspect, the present invention provides the compound, the conjugate, the vaccine, or the pharmaceutical composition, for use in preventing a P. aeruginosa infection.

In one aspect, the present invention provides an antibody, or an antigen binding fragment thereof, that selectively binds to the compound or the conjugate described herein, LPS of P. aeruginosa, and/or a cell of P. aeruginosa, wherein optionally the antibody or antigen binding fragment thereof is a monoclonal antibody or antigen binding fragment thereof.

In one aspect, the present invention provides an antibody, or an antigen binding fragment thereof, that selectively binds to an isolated oxidized A-band terminal epitope antigen (OS2), wherein optionally the antibody or antigen binding fragment thereof is a monoclonal antibody or antigen binding fragment thereof.

In one aspect, the present invention provides an antibody or antigen binding fragment thereof described herein, which is a chimeric or humanized antibody.

In one aspect, the present invention provides an antibody or antigen binding fragment thereof, wherein the antibody or antigen binding fragment thereof comprises a heavy chain variable domain comprising a variable heavy chain CDR1, a variable heavy chain CDR2, and a variable heavy chain CDR3,
wherein the variable heavy chain CDR1 comprises an amino acid sequence selected from the group consisting of SEQ ID NO: 1, SEQ ID NO: 10, and SEQ ID NO: 19;
wherein the variable heavy chain CDR2 comprises an amino acid sequence selected from the group consisting of SEQ ID NO: 2, SEQ ID NO: 11, and SEQ ID NO: 20; and
wherein the variable heavy chain CDR3 comprises an amino acid sequence selected from the group consisting of SEQ ID NO: 3, SEQ ID NO: 12, and SEQ ID NO: 21.

In one aspect, the present invention provides an antibody or antigen binding fragment thereof, comprising a light chain variable domain comprising a variable light chain CDR1, a variable light chain CDR2, and a variable light chain CDR3,
wherein the variable light chain CDR1 comprises an amino acid sequence selected from the group consisting of SEQ ID NO: 6, SEQ ID NO: 15, and SEQ ID NO: 24;
wherein the variable light chain CDR2 comprises an amino acid sequence selected from the group consisting of GTS, and RVS; and
wherein the variable light chain CDR3 comprises an amino acid sequence selected from the group consisting of SEQ ID NO: 7, SEQ ID NO: 16, and SEQ ID NO: 25.

In one aspect, the present invention provides an antibody or antigen binding fragment thereof, comprising a combination of a heavy chain variable domain (VH) and light chain variable domain (VL), wherein the combination is selected from the group consisting of:
a VH comprising the amino acid sequence of SEQ ID NO:4 and a VL comprising the amino acid sequence SEQ ID NO: 8;
a VH comprising the amino acid sequence of SEQ ID NO: 13 and a VL comprising the amino acid sequence SEQ ID NO: 17; and
a VH comprising the amino acid sequence of SEQ ID NO: 22 and a VL comprising the amino acid sequence SEQ ID NO: 26.

In one aspect, the present invention provides the antibody or antigen binding fragment thereof, for use in the treatment of a P. aeruginosa infection.

In one aspect, the present invention provides the antibody or antigen binding fragment thereof, for use in the diagnosis of a P. aeruginosa infection.

In one aspect, the present invention provides a method for treating a P. aeruginosa bacterial infection in an animal, comprising administering the antibody or antigen binding fragment thereof described herein to the animal.

In one aspect, the present invention provides a method for the diagnosis of a P. aeruginosa bacterial infection in an animal, comprising contacting a test sample with the antibody or antigen binding fragment thereof described herein, and detecting specific binding thereto.

In one aspect, the present invention provides a synthetic process to produce the compound of Formula A1, wherein m is 0, said process comprising:
anomeric deprotection of a 3-O-methylated rhamnopyranoside to form a 3-O-methylated rhamnopyranose;
acetylating the 3-O-methylated rhamnopyranose to form an acetylated 3-O-methylated rhamnopyranoside;
partially deprotecting 0-4 of the acetylated 3-O-methylated rhamnopyranoside to form a deprotected acetylated 3-O-methylated rhamnopyranoside;
coupling the 0-4 deprotected acetylated 3-O-methylated rhamnopyranoside to form an acetylated 3-O-methylated oligosaccharide;
deprotecting 0-4 of the acetylated 3-O-methylated oligosaccharide to form a 0-4 deprotected acetylated 3-O-methylated oligosaccharide; and
deacetylating the partially deprotected acetylated 3-O-methylated oligosaccharide to form the compound of Formula A1.

In one aspect, the present invention provides a synthetic process to produce the compound of Formula A1, wherein X is a handle, said process comprising the following steps:
glycosylating an activated O-3 methylated rhamnopyranoside intermediate at 1-O with a handle comprising a protected amine, wherein the activated monorhamnopyranoside intermediate comprises a protecting group at glycosylation site 4-O, and forming a protected 1-O glycosidic intermediate;
removing the protecting group from 4-O and forming a deprotected 1-O glycosidic intermediate;
coupling the deprotected 1-O glycosidic intermediate to an activated O-3 methylated rhamnopyranoside intermediate, wherein the activated rhamnopyranoside intermediate comprises a protecting group at 4-O, and forming a protected methylated disaccharide, trisaccharide, tetrasaccharide or pentasaccharide; and
removing all protecting groups from the protected oligosaccharide, wherein the oligosaccharide is preferably a trisaccharide, tetrasaccharide or pentasaccharide.

### BRIEF DESCRIPTION OF THE DRAWINGS

FIG. 1 shows structures of some of the isolated compounds described herein.

FIG. 2 shows ¹H NMR spectra of the band-A polysaccharide obtained from the LPS by alkaline treatment (lower trace), and its NaIO₄ oxidation product OS 1 (upper trace). * marks impurities, R—rhamnose H-1 from the rhamnan repeating units, Rm—anomeric signals of 3-OMe-Rha.

FIG. 3 shows overlap of the gCOSY (70% grey), TOCSY (50% grey) and ROESY (black) spectra of the A-PS, isolated from P. aeruginosa PAO1 wzy-mutant by mild acid hydrolysis of the LPS (anomeric region).

FIG. 4 shows positive ion mode ESI-MS spectrum of the OS 1. Two peaks labelled at m/z 1117.8 and 1122.6 correspond to ammonium and sodium adducts of Rha₅Man₁Tetritol-1d₁Me₆.

FIGS. 5A and 5B show ¹H-NMR spectrum of L-monosaccharide (FIG. 5A) and D-monosaccharide (FIG. 5B) recorded in CD₃OD.

FIG. 6 shows GC-MS traces of derived octyl glycosides of OS1 and standards.

FIG. 7 shows the structure of the Pseudomonas aeruginosa A-band 3-O-methyl D rhamnose pentasaccharide tip linked to 3-O-methyl D-mannose and glyceraldehyde prior to conjugation to CRIM.

FIGS. 8A, 8B, 8C, and 8D show MALDI-MS analyses of BSA (FIG. 8A), BSA-3-O-methyl rhamnan conjugate (FIG. 8B), CRM (FIG. 8C), and CRM-3-O-methyl rhamnan conjugate (FIG. 8D).

FIGS. 9A and 9B show ELISA titrations of D56 serum from mice (M1-M6) immunised with CRM-3-O-methyl rhamnan conjugate, upper curves vs. BSA-3-O-methyl rhamnan conjugate and lower curves vs PAO1 wt LPS with IgM antibodies left and IgG antibodies right (FIG. 9A) or D72 serum from rabbits (R1-R2) immunised with CRM-3-O-methyl rhamnan conjugate vs. PAO1 wt LPS left and vs. BSA-3-O-methyl rhamnan conjugate right (FIG. 9B).

FIG. 10 shows ELISA analysis of binding of 1B1, 3B8 and 3C4 mAb-containing hybridoma supernatant (used neat) to purified LPS antigens of P. aeruginosa strains PAO1wt, PAO1 (wzy::Gm)(Δpa5457) and PAO1 (wzy::Gm)(Δpa5458).

FIGS. 11A, 11B, and 11C show ELISA analysis of binding of 1B1 (FIG. 11A), 3B8 (FIG. 11B), and 3C4 (FIG. 11C) purified monoclonal antibodies (mAbs) (all at 100 μg/ml) to killed whole cells of P. aeruginosa strains NRCC #'s 6678, 6668-70, 6954-60 and negative control strains M. catarrhalis 6541 and N. meningitidis 6263 (see Table 1 for full details of strains).

FIG. 12 shows ELISA analysis of binding of 3C4 (panel a), 3B8 (panel b) and 1B1 (panel c) purified mAbs (all at 100 μg ml⁻¹) to killed whole cells of clinical isolates of P. aeruginosa strains NRCC #'s 6678, 6944-53 (see Table 1 for full details of strains).

FIGS. 13A and 13B show opsonophagocytic assay titration curves of mAbs 1B1 (solid line), 3B8 (dashed line) and 3C4 (dotted line) against P. aeruginosa strain PAO1 BAA-47 (FIG. 13A) and P. aeruginosa strain 537 (FIG. 13B).

FIG. 14A shows competition ELISAs between mAbs 1B1 and 3C4 for purified PAO1 LPS. mAb 1B1 was titered at the dilution shown on the x-axis onto an ELISA plate coated with PAO1 LPS. The plate was washed and mAb 3C4 was added at a constant concentration (62.5 μg/ml). A secondary mAb specific for mAb 3C4 was added and absorbance was measured after a colour reagent was added (line with square markers). The absorbance obtained when 3C4 was added to the plate without 1B1 competition is shown by the line with a triangle marker.

FIG. 14B shows competition ELISAs between mAbs 3B8 and 3C4 for purified PAO1 LPS. mAb 3B8 was titered at the concentrations shown on the x-axis onto an ELISA plate coated with PAO1 LPS. The plate was washed and mAb 3C4 was added at a constant concentration (62.5 μg/ml). A secondary mAb specific for mAb 3C4 was added and absorbance was measured after a colour reagent was added (line with square markers). The absorbance obtained when 3C4 was added to plate without 3B8 competition is shown by the line with a triangle marker.

FIGS. 15A, 15B, and 15C show inhibition ELISAs of mAbs 1B1 (FIG. 15A), 3C4 (FIG. 15B) and 3B8 (FIG. 15C) with LPS (left hand panels; square markers—P. aeruginosa PAO1 BAA-47, inverted triangle markers—P. aeruginosa (wzy::Gm)(Δpa5458), diamond markers—N. meningitidis galE/lpt3 and circular markers—PBS control) or synthetic oligosaccharides representing the terminal methylated rhamnan (right hand panels; solid circle markers—pentasaccharide, open square markers—tetrasaccharide, open circle markers—trisaccharide, inverted triangle markers—disaccharide, diamond markers—disaccharide with linker, triangle markers—L-monomer, solid square markers—D-monomer) against P. aeruginosa PAO1 BAA-47 LPS. Serial dilution, as shown on the x-axis, of either LPS or synthetic oligosaccharide was combined at an equal volume with 10 μg/ml mAb. After 1 h incubation, 100 μl of this inhibition mixture was added to PAO1 BAA-47 LPS coated plates. A secondary mAb specific for the primary mAb was added and absorbance was measured after a colour reagent was added. The absorbance obtained when the mAb is no longer inhibited by additional LPS or synthetic oligosaccharide is equal to inhibition by PBS.

FIGS. 15D, 15E, and 15F show inhibition ELISAs of mAbs 1B1 (FIG. 15D), 3C4 (FIG. 15E) and 3B8 (FIG. 15F) with LPS (left hand panels; square markers—P. aeruginosa PAO1 BAA-47, inverted triangle markers—P. aeruginosa (wzy::Gm)(Δpa5458), diamond markers—N. meningitidis galE/lpt3 and circle markers—PBS control) or synthetic oligosaccharides representing the terminal methylated rhamnan (right hand panels; solid circle markers—pentasaccharide, open square markers—tetrasaccharide, open circle markers—trisaccharide, inverted triangle markers—disaccharide, diamond markers—disaccharide with linker, triangle markers—L-monomer, blue—D-monomer) against P. aeruginosa PAO1 BAA-47 whole cells. Serial dilution, as shown on the x-axis, of either LPS or synthetic oligosaccharide was combined at an equal volume with 10 μg/ml mAb. After 1 h incubation, 100 μl of this inhibition mixture was added to PAO1 BAA-47 whole cell coated plates. A secondary mAb specific for primary the mAb was added and absorbance measured after a colour reagent was added. Absorbance obtained when the mAb is no longer inhibited by additional LPS or synthetic oligosaccharide is equal to inhibition by PBS.

FIG. 16 provides SPR sensorgrams showing binding of synthetic oligosaccharides to a high density 1B1 IgM surface. Various concentration ranges of synthetic oligosaccharides (see Experimental) were flowed over IgM 1B1 and an irrelevant IgM, Fn 4F1. Kinetics and affinities are reported in Table 5. Mcat lgt2/4: control oligosaccharide.

FIG. 17 shows an ¹H NMR spectrum for compound disaccharide (600 MHz, CD₃₀D).

FIG. 18 shows an ¹H NMR spectrum for compound trisaccharide (500 MHz, CD₃OD).

FIG. 19 shows an ¹H NMR spectrum for compound tetrasaccharide (500 MHz, CD₃OD).

FIG. 20 shows an ¹H NMR spectrum for compound pentasaccharide (600 MHz, CD₃OD).

FIG. 21 shows an ¹H NMR spectrum for compound disaccharide (600 MHz, D20).

FIG. 22 shows a reaction scheme for synthesis of D-monosaccharide (Scheme 1).

FIG. 23 shows a reaction scheme for synthesis of L-monosaccharide (Scheme 2).

FIG. 24 shows a reaction scheme for synthesis of 3-O-methyl D-rhamnose oligosaccharides (Scheme 3).

FIG. 25 shows a reaction scheme for addition of an aminoethyl handle to the disaccharide (Scheme 4).

FIG. 26A shows SDS-PAGE (left) and FIG. 26B Western blot (with mAb 1B1; right) analyses of HSA (lane 2) and HSA--3-O-methyl rhamnan pentasaccharide conjugate (two concentrations lanes 3 & 4). Molecular weight markers are shown in lane 1.

FIG. 27 shows MALDI-MS analyses of a) HSA, b) HSA-3-O-methyl rhamnan pentasaccharide conjugate.

FIGS. 28A, 28B, 28C, 28D, 28E, 28F, 28G, and 28H show ELISAs using mice sera from synthetic conjugate immunisation for ability to recognise P. aeruginosa LPS. Titrations of IgM and IgG antibodies at various timepoints are shown.

FIG. 29 shows ELISAs of synthetic oligosaccharide conjugate-derived antisera vs. killed whole cells.

FIG. 30 shows a reaction scheme for the synthesis of tri-, tetra-, and penta-saccharides including a handle and optionally a linker.

FIG. 31A shows NMR of 3-O-Methyl-α-D-rhamnopyranoside-(1→4)-3-O-methyl-α-D-rhamnopyranoside-(1→4)-2-(2,2-dimethoxybutylcarbonyl)amino]ethyl 3-O-methyl-α-D-rhamnopyranoside (S22), ¹H NMR, 600 MHz, CD₃₀D.

FIG. 31B shows NMR of 3-O-Methyl-α-D-rhamnopyranoside-(1→4)-3-O-methyl-α-D-rhamnopyranoside-(1→4)-3-O-methyl-α-D-rhamnopyranoside-(1→4)-2-(2,2-dimethoxybutylcarbonyl)amino]ethyl 3-O-methyl-α-D-rhamnopyranoside (S23), ¹H NMR, 600 MHz, CD₃₀D.

FIG. 32 shows NMR of 3-O-Methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranoside-(1→4)-2-(2,2-dimethoxybutylcarbonyl)amino]ethyl 3-O-methyl-α-D-rhamnopyranoside (S24), ¹H NMR, 600 MHz, CD₃₀D.

FIG. 33. Inhibition ELISA of mAb 1B1 with LPS (left-hand graph; P. aeruginosa PAO1 BAA-47 (wt); N. meningitidis galE lpt3; PBS control) or synthetic oligosaccharides with linker representing the terminal methylated rhamnan (right-hand graph; pentasaccharide; tetrasaccharide; trisaccharide; against P. aeruginosa PAO1 BAA-47 (wt) LPS. Serial dilution, as shown on the x axis.

FIG. 34A. ELISA determined recognition with pre-immune mice sera IgM (1:40 dilution) prior to CRM-oligosaccharide conjugate immunisation vs BSA-oligosaccharide conjugates. Mice MRha3V 1-5 will receive the trisaccharide CRM conjugate, mice MRha4V 6-10 will receive the tetrasaccharide CRM conjugate and mice MRha5V 11-15 will receive the pentasaccharide CRM conjugate.

FIG. 34B. ELISA determined recognition with pre-immune mice sera IgM titration prior to CRM-oligosaccharide conjugate immunisation vs Pa wt LPS. Mice MRha3V 1-5 will receive the trisaccharide CRM conjugate, mice MRha4V 6-10 will receive the tetrasaccharide CRM conjugate and mice MRha5V 11-15 will receive the pentasaccharide CRM conjugate.

FIG. 35A. ELISA determined recognition with final bleed mice sera IgM titration following CRM-oligosaccharide conjugate immunisation vs BSA-trisaccharide conjugates. Mice MRha3V 1-5 received the trisaccharide CRM conjugate, mice MRha4V 6-10 received the tetrasaccharide CRM conjugate and mice MRha5V 11-15 received the pentasaccharide CRM conjugate.

FIG. 35B. ELISA determined recognition with final bleed mice sera IgM titration following CRM-oligosaccharide conjugate immunisation vs BSA-tetrasaccharide conjugates. Mice MRha3V 1-5 received the trisaccharide CRM conjugate, mice MRha4V 6-10 received the tetrasaccharide CRM conjugate and mice MRha5V 11-15 received the pentasaccharide CRM conjugate.

FIG. 35C. ELISA determined recognition with final bleed mice sera IgM titration following CRM-oligosaccharide conjugate immunisation vs BSA-pentasaccharide conjugates. Mice MRha3V 1-5 received the trisaccharide CRM conjugate, mice MRha4V 6-10 received the tetrasaccharide CRM conjugate and mice MRha5V 11-15 received the pentasaccharide CRM conjugate.

FIG. 35D. ELISA determined recognition with final bleed mice sera IgM titration following CRM-oligosaccharide conjugate immunisation vs LPS. Mice MRha3V 1-5 received the trisaccharide CRM conjugate, mice MRha4V 6-10 received the tetrasaccharide CRM conjugate and mice MRha5V 11-15 received the pentasaccharide CRM conjugate.

FIG. 36A. ELISA determined recognition with pre-immune mice sera IgG (1:40 dilution) prior to CRM-oligosaccharide conjugate immunisation vs BSA-oligosaccharide conjugates. Mice MRha3V 1-5 will receive the trisaccharide CRM conjugate, mice MRha4V 6-10 will receive the tetrasaccharide CRM conjugate and mice MRha5V 11-15 will receive the pentasaccharide CRM conjugate.

FIG. 36B. ELISA determined recognition with pre-immune mice sera IgG titration prior to CRM-oligosaccharide conjugate immunisation vs Pa wt LPS. Mice MRha3V 1-5 will receive the trisaccharide CRM conjugate, mice MRha4V 6-10 will receive the tetrasaccharide CRM conjugate and mice MRha5V 11-15 will receive the pentasaccharide CRM conjugate.

FIG. 37A. ELISA determined recognition with final bleed mice sera IgG (1:20 dilution) following CRM-oligosaccharide conjugate immunisation vs BSA-oligosaccharides and Pa wt LPS as indicated. Mice MRha3V 1-5 received the trisaccharide CRM conjugate, mice MRha4V 6-10 received the tetrasaccharide CRM conjugate and mice MRha5V 11-15 received the pentasaccharide CRM conjugate. Pre-immune sera (Pre) was included as a negative control and mAb 3C4 (isotype IgG2b) was included as a positive control for Pa wt LPS.

FIG. 37B. ELISA determined recognition with pooled pre- and final bleed mice sera IgG (1:40 dilution) following CRM-oligosaccharide conjugate immunisation vs Pa wt, Pa wzy5457, Pa wzy5458 and Nm wt (negative control) LPS as indicated. Mice MRha3V 1-5 received the trisaccharide CRM conjugate, mice MRha4V 6-10 received the tetrasaccharide CRM conjugate and mice MRha5V 11-15 received the pentasaccharide CRM conjugate. Pre-immune sera (Pre) was included as a negative control.

FIG. 38A. ELISA determined recognition with pre- and post-immune (D70) rabbit sera (1:500 and 1:1500 dilution) prior to and following CRM-oligosaccharide conjugate immunisation vs BSA-oligosaccharide conjugates. Rabbits RRha3V 1-2 received the trisaccharide CRM conjugate, rabbits RRha4V 3-4 received the tetrasaccharide CRM conjugate and rabbits RRha5V 5-6 received the pentasaccharide CRM conjugate.

FIG. 38B. ELISA determined recognition with pre- and post-immune (D70) rabbit sera titration prior to and following CRM-oligosaccharide conjugate immunisation vs Pa wt, Pa wzy5457 and Pa wzy5458 LPS. Rabbits RRha3V 1-2 received the trisaccharide CRM conjugate, rabbits RRha4V 3-4 received the tetrasaccharide CRM conjugate and rabbits RRha5V 5-6 received the pentasaccharide CRM conjugate.

FIG. 39A. ELISA analysis of binding of pooled mice sera pre- and post-immune (IgM and IgG all at 1:40 dilution) to killed whole cells of P. aeruginosa strains NRCC #'s 6678, 6667-70, 6954-60 and negative control strain M. catarrhalis 6541 (see Table 7 for full details of strains).

FIG. 39B. ELISA analysis of binding of individual rabbit sera pre- and post-immune (all at 1:1500 dilution) to killed whole cells of P. aeruginosa strains NRCC #'s 6678, 6667-70, 6954-60 and negative control strain M. catarrhalis 6541 (see Table 7 for full details of strains).

FIG. 39C. ELISA analysis of binding of individual rabbit sera pre- and post-immune (all at 1:1500 dilution) to killed whole cells of clinical P. aeruginosa strains NRCC #'s 6678, 6944-46, 6948-53 and negative control strains N. meningitidis 6263 and M. catarrhalis 6541 (see Table 7 for full details of strains).

### DETAILED DESCRIPTION

The present invention is based, in part, on the identification of novel P. aeruginosa polysaccharide structures by NMR and chemical analysis. It is believed that the structures provided herein are the first identification or the first correct identification of P. aeruginosa A-band terminal epitope antigen (A-PS).

Pseudomonas aeruginosa (also referred to as P. aeruginosa) is an opportunistic bacterial pathogen and the etiologic agent of several potentially life-threatening infections, including healthcare-associated and ventilator-associated pneumonia, chronic pulmonary infection in cystic fibrosis (CF) patients, and burn and soft tissue infections. There is an increasing prevalence of drug-resistant P. aeruginosa infections. Thus, in some examples, the term “P. aeruginosa” refers to a pathogenic strain of Pseudomonas aeruginosa, including, but not limited to, antibiotic-resistant strains, such as P. aeruginosa strains resistant to β-lactam antibiotics (e.g., penicillin), piperacillin, imipenem, tobramycin or ciprofloxacin. In some embodiments, the term “P. aeruginosa” refers to a pathogenic strain that infects cystic fibrosis patients.

The term “infection” refers to any microbial infection of a subject's body. Infection includes the invasion of a patient's body by a microbe and subsequent replication of the microbe in the subject's body. In a specific example, the microbe is P. aeruginosa.

The term “subject”, as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. In a specific example, the subject is a human.

### Glycans

As used herein, the terms ““carbohydrate”, glycan”, “saccharide”, “oligosaccharide”, and “polysaccharide”, are used interchangeably and refer to oligomers or polymers made up of sugar monomers, typically joined by glycosidic bonds also referred to herein as linkages. Within a glycan, monosaccharide monomers may all be the same or they may differ. Common monomers include, but are not limited to trioses, tetroses, pentoses, glucose, fructose, galactose, rhamnose and 3-O-methyl rhamnose, xylose, arabinose, lyxose, allose, altrose, mannose and 3-O-methyl mannose, gulose, iodose, ribose, mannoheptulose, sedoheptulose and talose. Amino sugars may also be monomers within a glycan. Glycans comprising such sugars are herein referred to as aminoglycans. Amino sugars, as used herein, are sugar molecules that comprise an amine group in place of a hydroxyl group, or in some embodiments, a sugar derived from such a sugar. Examples of amino sugars include, but are not limited to glucosamine, galactosamine, N-acetylglucosamine, N-acetylgalactosamine, sialic acids (including, but not limited to, N-acetylneuraminic acid and N-glycolylneuraminic acid) and L-daunosamine.

In some cases, glycans may be modified with one or more non-glycan components including, but not limited to labels, handles, linkers, spacers, carriers, and the like. In some embodiments, glycans may comprise glycoconjugates. Glycoconjugates may include, but are not limited to glycoproteins, glycolipids or proteoglycans. Glycoproteins include any proteins that contain covalently attached oligosaccharide chains (glycans). Unless otherwise specified, the polysaccharide nomenclature used herein follows the IUB-IUPAC Joint Commission on Biochemical Nomenclature (JCBM) Recommendations 1980. See JCBN, 1982, J. Biol. Chem. 257:3352-3354.

The label “D-monosaccharide” as used herein, e.g. in the Figures, refers specifically to 3-O-methyl-D-rhamnose, and the label “L-monosaccharide” as used herein, e.g. in the Figures, refers specifically to 3-O-methyl-L-rhamnopyranose, and the label “disaccharide” as used herein, e.g. in the Figures, refers specifically to: 3-O-methyl-α-D-rhamnopyranoside-(1→4)-3-O-methyl-D-rhamnopyranose, and as will be evident from the context.

The term “antigen” as used herein, refers to a substance capable of initiating and mediating an immune response. The immune responses stimulated by antigens may be one or both of humoral or cellular, and generally are specific for the antigen. Antigens that stimulate or potentiate immune responses are said to be immunogenic and may be referred to as immunogens. Compositions comprising antigens may be referred to as “antigenic compositions” or “immunogenic compositions”

Accordingly, antigens are substances that may be bound by antibody molecules or by T cell receptors. Many types of biological and other molecules can act as antigens. For example, antigens may originate from molecules that include, but are not limited to, proteins, peptides, carbohydrates, polysaccharides, oligosaccharides, sugars, lipids, phospholipids, glycophospholipids, and other molecules, and fragments and/or combinations thereof.

Antigens may originate from innate sources or from sources extrinsic to a particular mammal or other animal (e.g., from infectious agents). Antigens may possess multiple antigenic determinants such that exposure of a mammal to an antigen may produce a plurality of corresponding antibodies or cellular immune responses with differing specificities.

As noted above, in a specific example, there is described an isolated or chemically synthesised glycan antigen from P. aeruginosa.

The antigen may serve to sensitize the host by the presentation of the antigen in association with MHC molecules at a cell surface. In addition, antigen-specific T-cells or antibodies can be generated to allow for the future protection of an immunized host. Immunogenic compositions thus can protect the host from infection by the bacteria, reduced severity, or may protect the host from death due to the bacterial infection. Antigens may also be used to generate polyclonal or monoclonal antibodies, which may be used to confer passive immunity to a subject. Antigens may also be used to generate antibodies that are functional as measured by the killing of bacteria in either an animal efficacy model or via an opsonophagocytic killing assay.

As used herein, the term “isolated” in connection with a polysaccharide refers to isolation of A-band terminal epitope antigen (A-PS) from purified polysaccharide using purification techniques known in the art, including the use of centrifugation, depth filtration, precipitation, ultrafiltration, treatment with activate carbon, diafiltration and/or column chromatography. Generally an isolated polysaccharide refers to partial removal of proteins, nucleic acids and non-specific endogenous polysaccharide. The isolated polysaccharide contains less than 10%, 8%, 6%, 4%, or 2% protein impurities and/or nucleic acids.

As used herein, the term “purified” in connection with a bacterial polysaccharide refers to the purification of the polysaccharide from cell lysate through means such as centrifugation, precipitation, and ultra-filtration. Generally, a purified polysaccharide refers to removal of cell debris and DNA.

Bacterial glycans may be derived from naturally-occurring bacteria, genetically engineered bacteria, or can be produced synthetically. The polysaccharides are typically subjected to one or more processing steps prior to use, for example, purification, functionalization, depolymerization using mild acidic or oxidative conditions, deacetylation, and the like. Post processing steps can also be employed, if desired. Any suitable method known in the art for synthesizing, preparing, and/or purifying suitable polysaccharides and oligosaccharides can be employed.

Pseudomonas aeruginosa produces a variety of cell surface glycans. Previous studies have identified a common polysaccharide (PS) antigen often termed A-band PS that is composed of a neutral D-rhamnan trisaccharide repeating unit as a relatively conserved cell surface carbohydrate. One study showed no immunogenic effect from a neutral D-rhamnan triscaccharide [25]. As mentioned above, the present inventors have identified novel P. aeruginosa polysaccharide structures and shown that the carbohydrate antigen consists of an immunogenic methylated rhamnan oligosaccharide at the non-reducing end of the A-band PS:

α-D-Rha3OMe-4-(α-D-Rha3OMe-4-)₄—

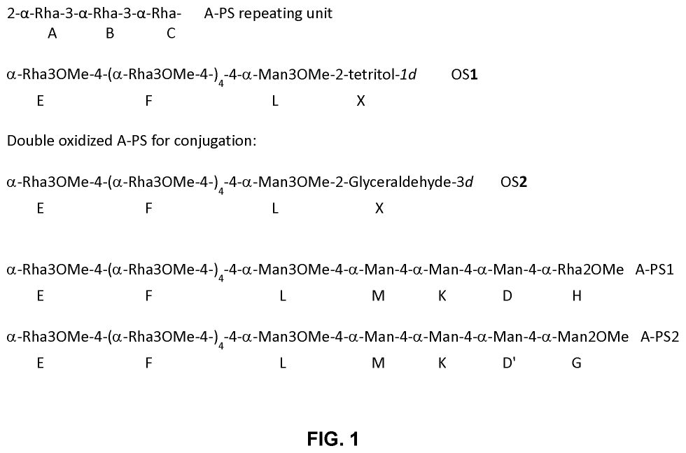

In particular the inventors isolated and characterized A-PS1 and A-PS2 (see FIG. 1). It is believed that the structures provided herein are the first identification or the first correct identification of P. aeruginosa A-band terminal epitope antigen. The A-PS tip (see A-PS1 and A-PS2 in FIG. 1) was further isolated to its antigenic component to produce OS1 and OS2. It was determined that the methylate rhamnose moieties provided the antigenicity. Di,-tri, -tetra, and -penta 3 OMe rhamose oligosaccharides were synthesized with and without a handle at the reducing end.

In one aspect, the present invention thus provides an antigenic compound comprising the oligosaccharide moiety of Formula A:

α-Rha3OMe(-4α-Rha3OMe)ₙ- Formula A

wherein n is 1-5, preferably 2-4, and wherein the 2-position in each Rha3OMe saccharide moiety is independently substituted with —OAc or —OH.

In one aspect, the present invention provides an antigenic compound comprising the oligosaccharide moiety of Formula A1:

α-Rha3OMe(-4α-Rha3OMe)ₙ-X Formula A1

wherein n is 1-5 (preferably 2-4), and X is —H or -(4α-Man3OMe)ₘ-handle; and m is 0, 1, or 2, preferably 0 or 1; and wherein the 2-position in each Rha3OMe saccharide moiety is independently substituted with —OAc or —OH.

The saccharide monomeric moieties may be independently in the D or the L configuration.

In some examples, the saccharidic moieties may be independently acetylated at 2-0. In other examples, an alternating pattern of 3-O-methyl rhamnose acetylated and non-acetylated may be used. In other examples, 2-0 may be substituted with groups other than acetates to improve immunogenicity, such as glycolyl and lactyl.

Specific examples of methylated rhamnose oligosaccharides include:
α-D-Rha3OMe-4-(α-D-Rha3OMe-4)₄-4-α-D-Man3OMe-2-glyceraldehyde-1d (OS2);
3-O-methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranose (pentasaccharide);
3-O-methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranose (tetrasaccharide) (II);
3-O-methyl-α-D-rhamnopyranoside-(1→4)-3-O-methyl-α-D-rhamnopyranoside-(1→4)-3-O-methyl-α-D-rhamnopyranose (trisaccharide)
3-O-methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranoside-(1→4)-4-3-O-methyl-α-D-mannopyranoside

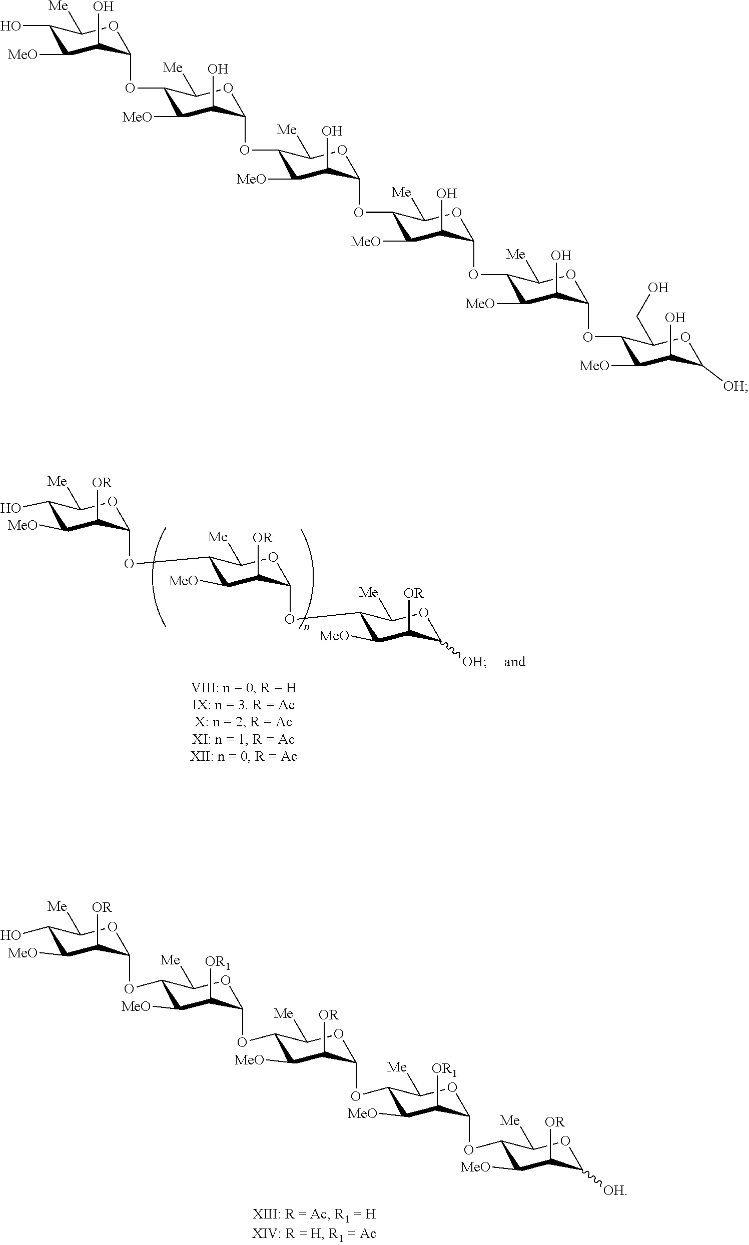

The methylated rhamnose oligosaccharides of the invention can be isolated or synthesized chemically. Alternatively the isolate oligosaccharides can be further modified chemically.

Isolation of A-band terminal epitope antigen: The present inventors have developed a method for isolating an A-band terminal epitope antigen (A-PS). In one aspect, there is provided a method for producing an A-band terminal epitope antigen (OS1) comprising subjecting isolated LPS (lipopolysaccharide) from P. aeruginosa to acid or alkaline hydrolysis, to produce a polymeric fraction. In one example, the polymeric fraction is subject to acid hydrolysis, such as with acetic acid. In one example, the polymeric fraction is subject to alkaline hydrolysis, such as by reaction with KOH, followed by treatment with HCl.

The polymeric fraction is then subject to an oxidation step followed by acid hydrolysis to produce OS1. In one example, said first oxidation step comprises: reacting said polymeric fraction with NaIO₄, reacting with ethylene glycol and NaBD₄, reacting with AcOH, and desalting to produce a product, and subjecting the product to acid hydrolysis, to produce the isolated A-band terminal epitope antigen (OS1). In a specific example, there is described an isolated A-band terminal epitope antigen (OS1) from P. aeruginosa, having the following formula:

α-D-Rha3OMe-4-(α-D-Rha3OMe-4)₄-4-α-D-Man3OMe-2-tetritol-1d (OS1)

If OS1 is subject to a further oxidation step, a further isolated A-band terminal epitope is obtained, identified herein as OS2. In one example, said second oxidation step comprises: reacting OS1 with NaIO₄, reacting with ethylene glycol and NaBD₄, reacting with AcOH, and desalting to produce a product, and subjecting the product to acid hydrolysis, to produce the isolated A-band terminal epitope antigen (OS2). In a specific example, there is described an isolated oxidized A-band terminal epitope antigen (OS2)P. aeruginosa, which is a glycan that is a compound of Formula:

α-D-Rha3OMe-4-(α-D-Rha3OMe-4)₄-4-α-D-Man3OMe-2-glyceraldehyde-1d (OS2)

In some examples, the compound is an antigen.

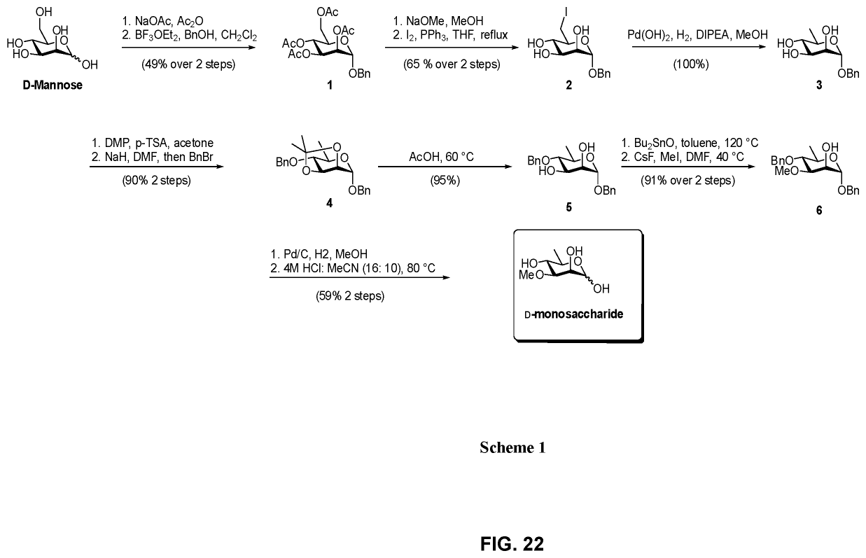

Chemical Synthesis: The chemical synthesis of the compounds of the invention is described in detail in the Examples and is illustrated in FIGS. 22-24 and 30.

In one aspect there is provided a synthetic process to produce a compound of the invention, the process comprising:
anomeric deprotection of a 3-O-methylated rhamnopyranoside to form a 3-O-methylated rhamnopyranose;
acetylating the 3-O-methylated rhamnopyranose to form an acetylated 3-O-methylated rhamnopyranoside;
partially deprotecting O-4 of the acetylated 3-O-methylated rhamnopyranoside to form a deprotected acetylated 3-O-methylated rhamnopyranoside;
coupling the O-4 deprotected acetylated 3-O-methylated rhamnopyranoside to form an acetylated 3-O-methylated oligosaccharide;
deprotecting O-4 of the acetylated 3-O-methylated oligosaccharide to form a O-4 deprotected acetylated 3-O-methylated oligosaccharide; and
deacetylating the partially deprotected acetylated 3-O-methylated oligosaccharide to form the compound.

In one aspect there is provided a synthetic process to produce a compound of the invention, the process comprising:

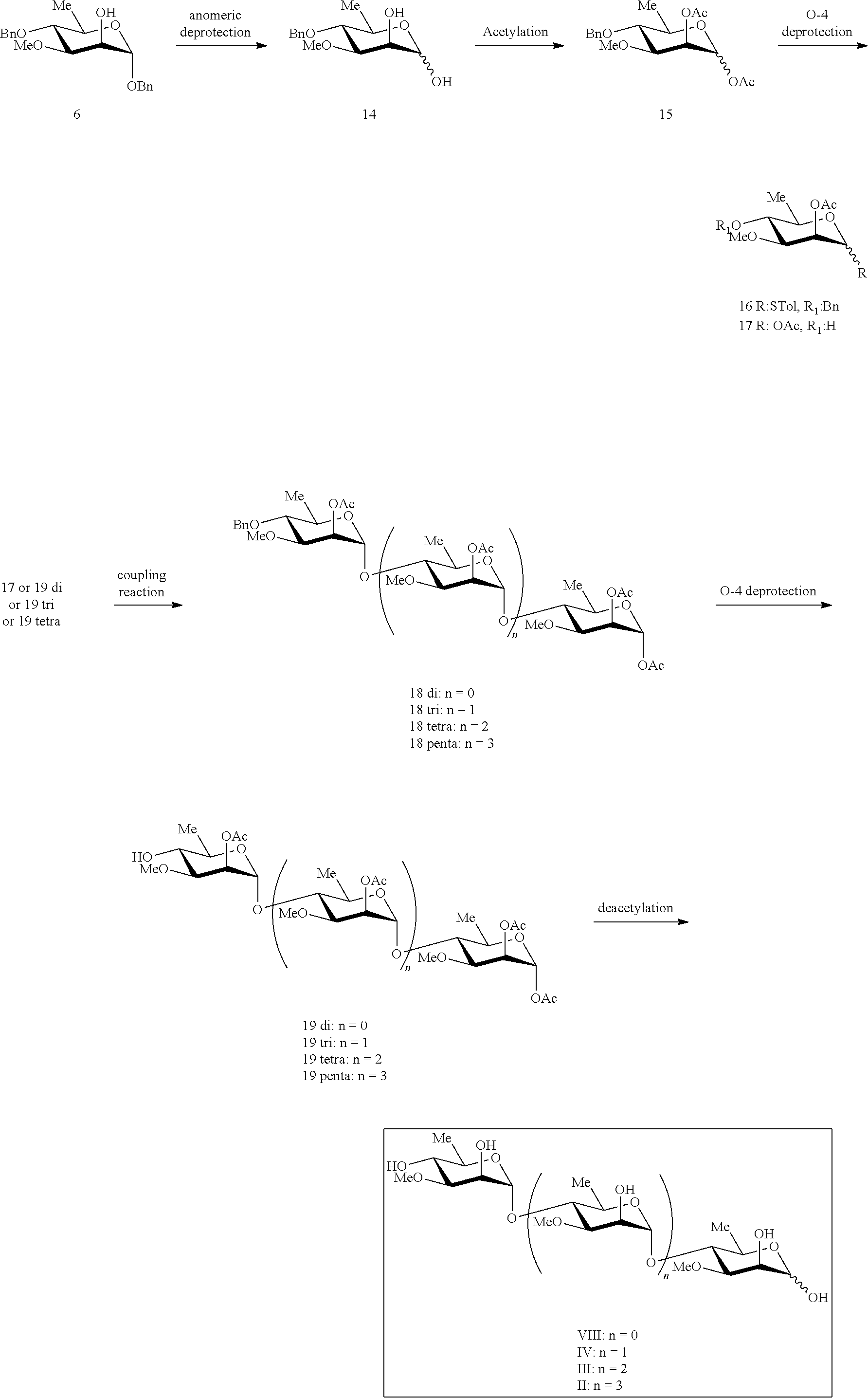

Handles: The compounds of the present invention also include compounds that include a “handle”. A “handle” in the context of the present invention is a chemical modification at a site distal to the terminal α-Rha3OMe repeat to form a reactive group. Examples include 2-glyceraldehyde, —CH₂—NH₂, —(CH₂)₂NH₂, —(CH₂)₃NH₂, —(CH₂)₄NH2, (CH2)₅NH2, —C(O)OH, —C(O)H, —C(O)NH₂, and —CH₂N₃. When the compound includes -4α-Man3OMe (i.e. m is 1 or 2), the handle is preferably 2-glyceraldehyde. When the compound does not include 4α-Man3OMe (i.e. m is 0), the handle is preferably —CH₂—NH₂, —(CH₂)₂NH₂, —(CH₂)₃NH₂, —(CH₂)₄NH2, (CH2)₅NH2, —C(O)OH, —C(O)H, —C(O)NH₂, and —CH₂N₃, more preferably —CH₂—NH₂, —(CH₂)₂NH₂, —(CH₂)₃NH₂, —(CH₂)₄NH2, or —(CH2)₅NH2.

In one example, the synthetic process further comprises adding a handle by performing the following steps:
glycosylating an activated O-3 methylated rhamnopyranoside intermediate at 1-O with a handle comprising a protected amine, wherein the activated monorhamnopyranoside intermediate comprises a protecting group at glycosylation site 4-O, and forming a protected 1-O glycosidic intermediate;
removing the protecting group from 4-O and forming a deprotected 1-O glycosidic intermediate;
coupling the deprotected 1-O glycosidic intermediate to an activated O-3 methylated rhamnopyranoside intermediate, wherein the activated rhamnopyranoside intermediate comprises a protecting group at 4-O, and forming a protected methylated disaccharide, trisaccharide, tetrasaccharide or pentasaccharide; and
removing all protecting groups from the protected disaccharide, trisaccharide, tetrasaccharide or pentasaccharide.

In one example, the synthetic process further comprises adding a handle to a compound of the invention, by performing the following steps:

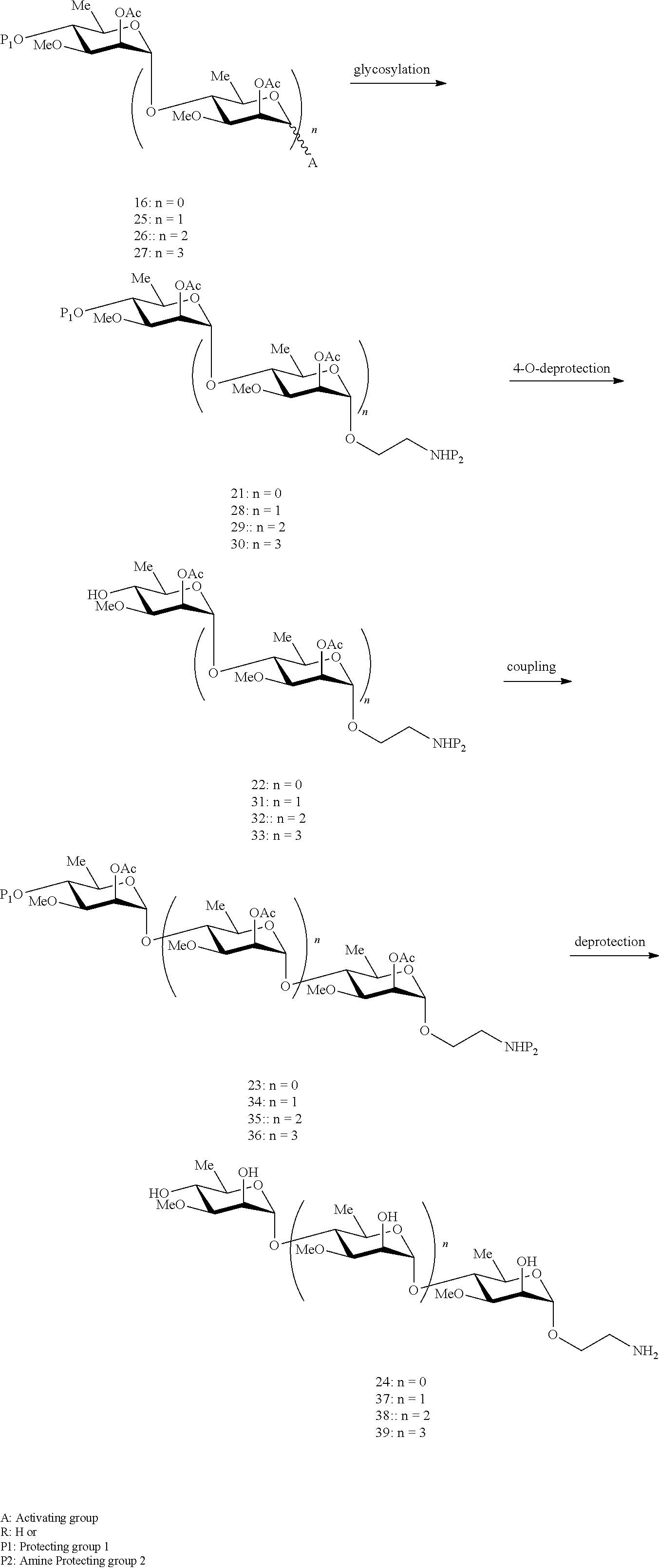

Linkers: The purified or synthesized oligosaccharides can optionally comprise a linker, which may be conjugated to the oligosaccharides of the invention directly or through a handle on the oligosaccharide. As will be appreciated by one of skill in the art, the linker may be any suitable linker for the desired purpose, for example, for conjugation of the 4α-linked glycan to the carrier protein. Suitable linkers, containing functional groups on both ends, such as an acid, or an NHS ester, or a PFP ester, will be known to one skilled in the art and include, but are not limited to, for example, polyethylene glycol (PEG), linear poly-amidoamine (PAA), poly(2-oxazoline)s (POx), poly (glycerol adipate) (PGA), polyhydroxyalkanoates (PHA), and other linkers suitable for the preparation of glycoconjugates, such as those described in Munneke et al [42]. Examples of suitable linkers are obtainable, for example from BroadPharm® as BCN PEG and BCN Reagents.

In certain embodiments, the oligosaccharide can be coupled to a linker to form a polysaccharide-linker in which the free terminus of the linker is an ester group. The linker is therefore one in which at least one terminus is an ester group. The other terminus is selected so that it can react with the oligosaccharide to form the oligosaccharide-linker intermediate.

In certain embodiments, the linker is a bifunctional linker that provides a first ester group for reacting with the primary amine group on the handle of the oligosaccharide and a second ester group for reacting with the primary amine group in the carrier molecule. A typical linker is adipic acid N-hydroxysuccinimide diester (SIDEA). In one aspect, the linker may be a bivalent linker containing an activated N-hydroxysuccinimide and a hemiacetal protected aldehyde, wherein one end is reactive with the primary amine on the handle of the oligosaccharided.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with CDI (See Bethell et al., [43]) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In some examples, a linker may be used for linking at the anomeric position. In some examples, the linker may be placed early during the synthetic process. It may be placed on the disaccharide, trisaccharide, tetrasaccharide or pentasaccharide or additionally to the acetylated versions and the pattern of non-acetylated/acetylated pattern of these saccharides. In other examples, it may be possible to link the oligosaccharide to any linking functionalization for potential conjugation to a protein.

In one aspect, a linker such as amino-peg and a functional group such as azide and aldehyde protecting group may be used.

In one example, a linker may be added according to the following process:

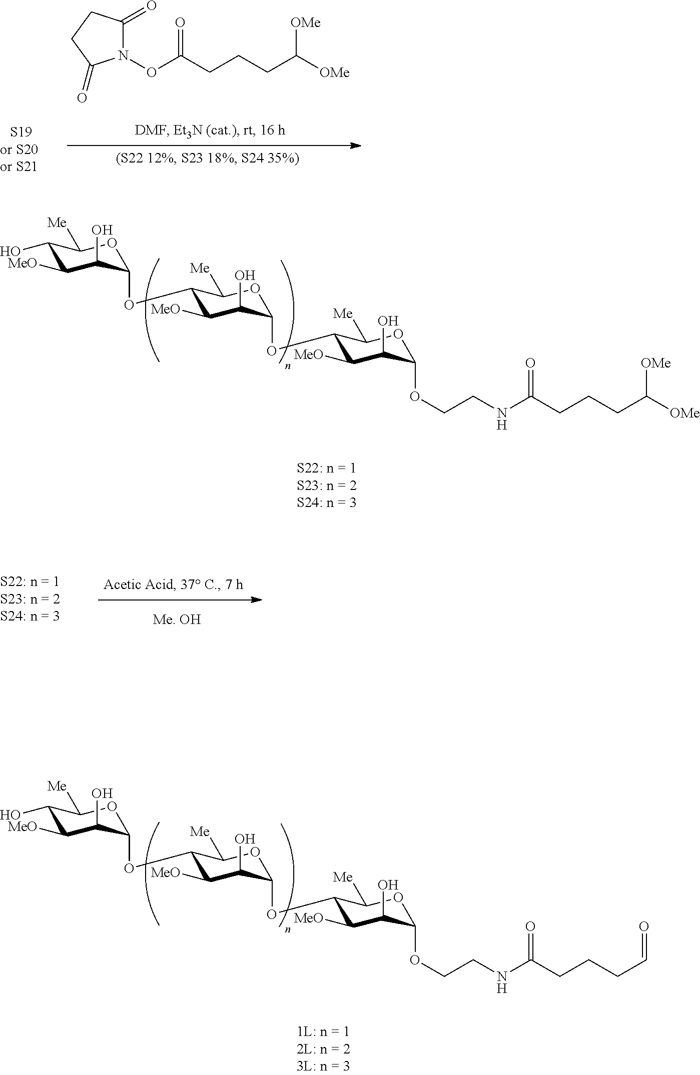

Conjugates: Conjugation to a carrier protein can improve immunogenicity. Suitable classes of proteins include pili, outer membrane proteins and excreted toxins of pathogenic bacteria; nontoxic or “toxoid” forms of such toxins, nontoxic proteins antigenically similar to bacterial toxins (i.e. cross-reacting materials or CRMs) and other proteins. In one aspect, CRM, such as CRM197 can be used as a carrier protein. CRM197 is a non-toxic variant of diphtheria toxin (DT). Other suitable carrier proteins include additional inactivated bacterial toxins such as DT, Diphtheria toxoid fragment B (DTFB), DTB C8, TT (tetanus toxid) or fragment C of TT, pertussis toxoid, cholera toxoid, E. coli LT (heat-labile enterotoxin), E. coli ST (heat-stable enterotoxin), and a Pseudomonas aeruginosa protein such as exotoxin A from Pseudomonas aeruginosa. Also included are human serum albumin (HSA) and bovine serum albumin (BSA). Other DT mutants can also be used as the carrier protein, such as CRM176, CRM228, CRM45; CRM9, CRM45, CRM102, CRM103, CRM3201, and CRM107. Also included are Clostridium perfringens exotoxins/toxoid. Other suitable bacterial proteins include, but are not limited to, pneumococcal surface protein A (PspA), pneumococcal adhesin protein (PsaA), and pneumococcal surface proteins BVH-3 and BVH-11. The use of immunogenic carrier proteins from non-mammalian sources including keyhole limpet hemocyanin (KLH), horseshoe crab hemocyanin and plant edestin is also contemplated, as is the use of viral proteins such as hepatitis B surface/core antigens; rotavirus VP7 protein and respiratory syncytial virus F and G proteins. Other carrier proteins will be known to those of skill in the art.

The glycan conjugates may be prepared by known coupling techniques. The oligosaccharides of the invention may be conjugated directly or through a handle and/or linker to a carrier protein to form a glycoconjugate, using chemistry discussed above with respect to the linkers.

Conjugation between the glycan and the carrier may be achieved using a variety of reagents. The conjugation may be directly between the glycan and the carrier protein, such as a direct covalent linkage by reductive amination. Alternatively, conjugation may be carried out using a cross-linking agent.

In one example, the protein may be conjugated to a glycan-handle-linker compound per the following:

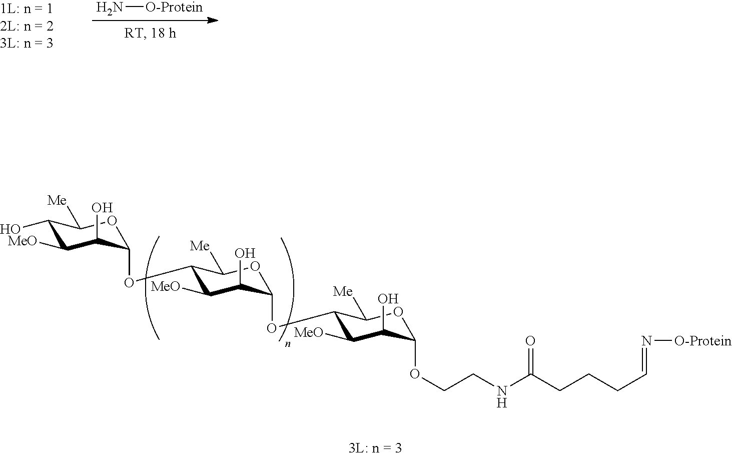

Further examples of protein and linker conjugation are shown below.

In some examples, the antigen is the natural saccharide extracted and modified from Pseudomonas aeruginosa (OS2), which may be conjugated to a carrier protein such as CRM, HSA, BSA, or the like. In this case, glyceraldehyde acts as a handle, and the protein is attached via a reductive amination reaction.

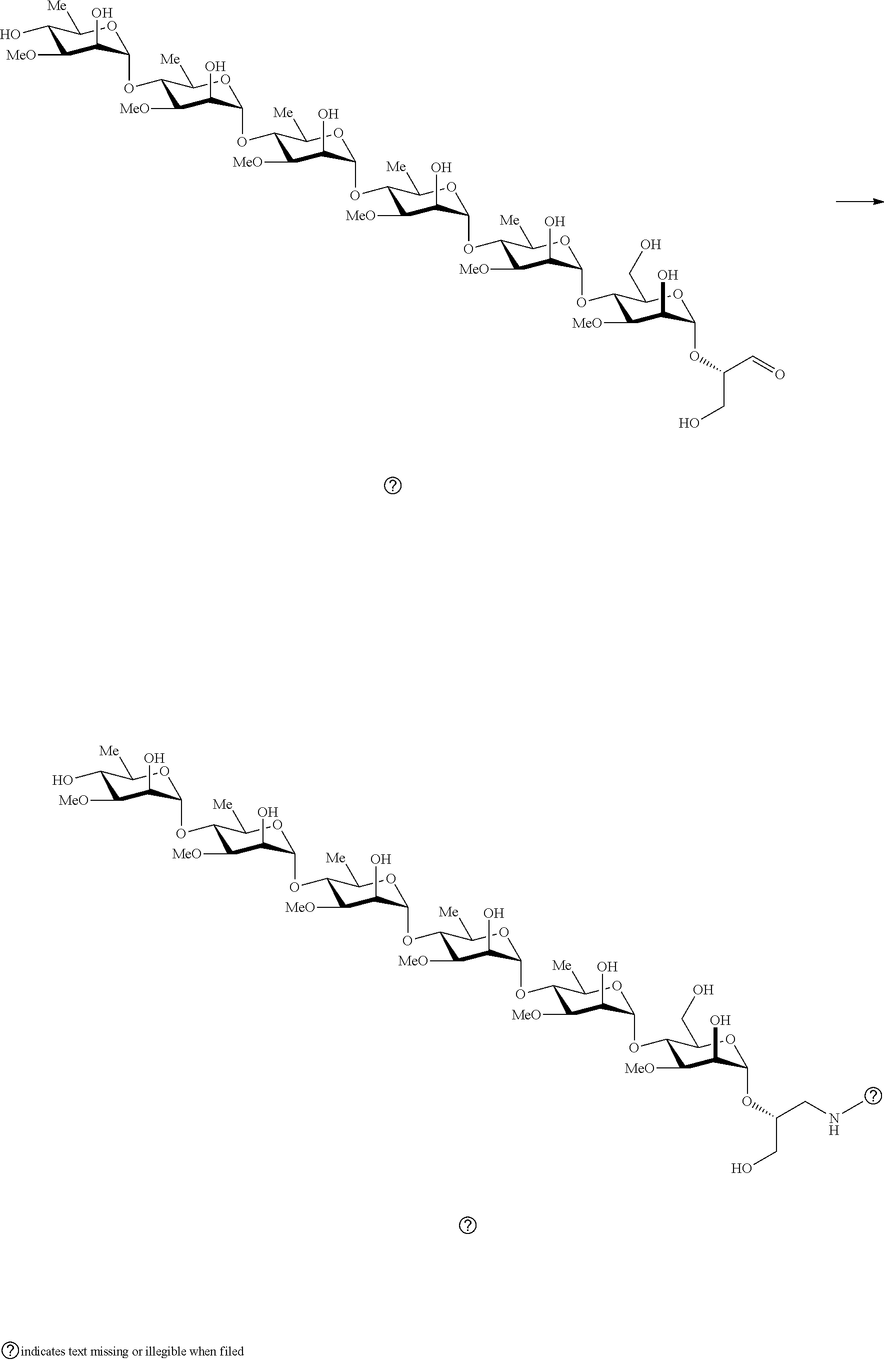

In some examples, the antigen comprises or consists of

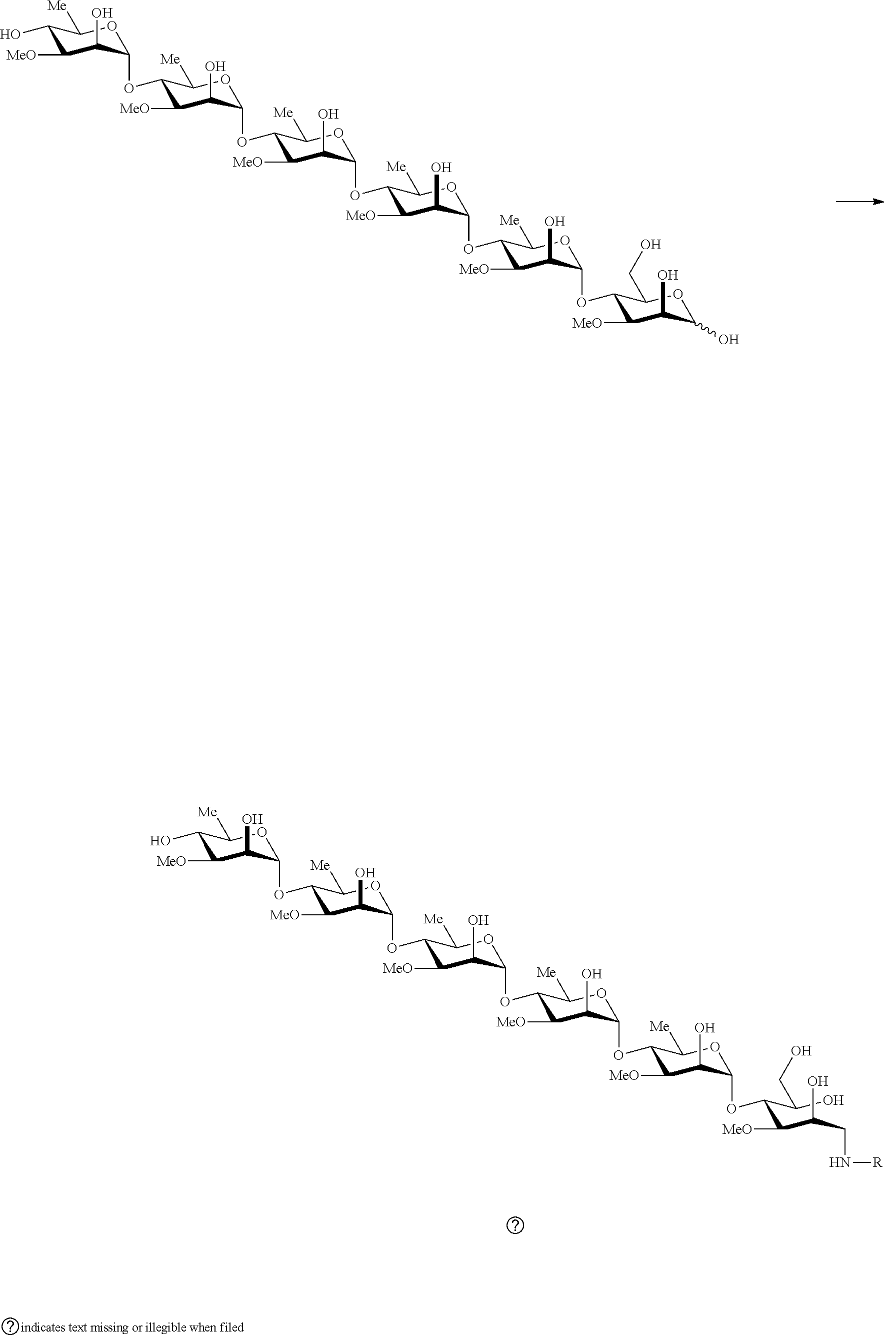

In some examples, direct reductive amination of the hemiacetal with a protein, as an example, may be used. In some examples this is achieved via the reducing end of the synthetic antigen. In some examples, a linker may first be added to the saccharide via the handle and conjugate to the attached linker with known linking technology.

In one aspect, the carrier protein or the linker can be conjugated by direct reductive amination with the amines from the carrier protein or linker, respectively (R is the protein or linker). The following is an example.

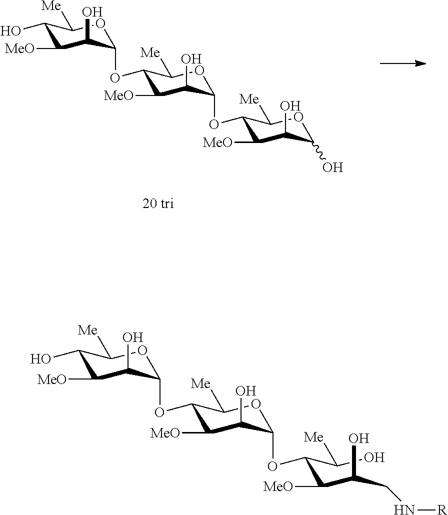

In some examples, the oligosaccharides may be acetylated at 2-O.

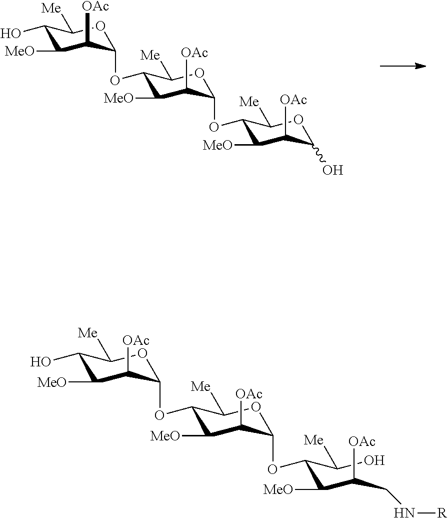

In other examples, a combination of non-acetylated and acetylated monosaccharides may be used. In other examples, an alternating patter of 3-O-methyl rhamnose acetylated and nonacetylated, may be used.

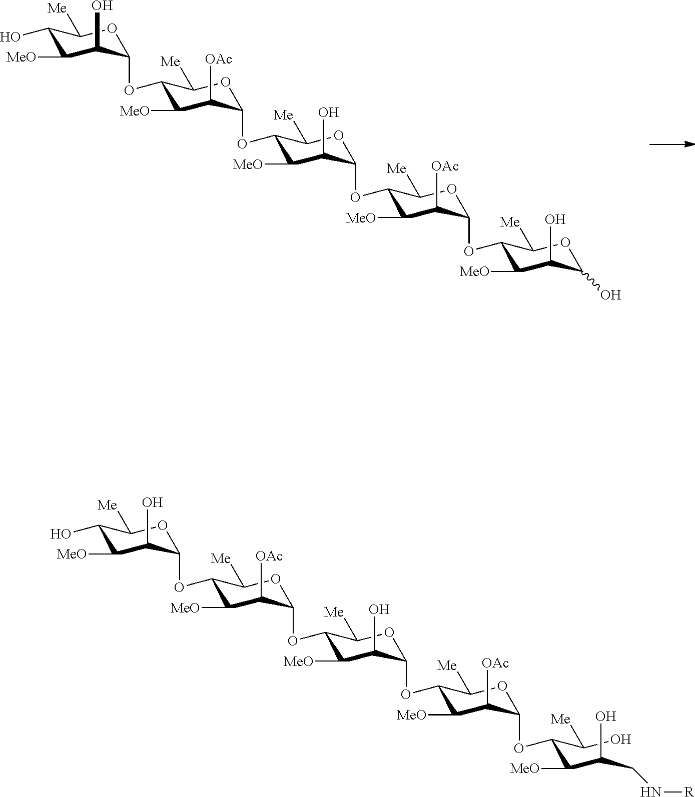

While the above illustrate specific compounds, the chemistry also applies to the other oligosaccharides of the invention, including the corresponding pentasaccharide, tetrasaccharide, trisaccharide, and disaccharides.

### Pharmaceutical/Vaccine Compositions

The present invention further provides compositions, including pharmaceutical, immunogenic and vaccine compositions, comprising, consisting essentially of, or alternatively, consisting of any of the glycans described herein including both those conjugated or non-conjugated to a carrier protein, together with a pharmaceutically acceptable carrier, excipient, and/or an adjuvant.

Formulation can be accomplished using art-recognized methods. For instance, the glycans can be formulated with a physiologically acceptable vehicle to prepare the composition. Examples of such vehicles include, but are not limited to, water, buffered saline, polyols (e.g., glycerol, propylene gly col, liquid polyethylene gly col) and dextrose solutions.

In some aspects, there is described a composition comprising a conjugate and a pharmaceutically acceptable excipient. In some aspects, there is described a composition comprising a conjugate, a pharmaceutically acceptable excipient, and an adjuvant.

In some examples, there is described a vaccine, the glycan being an antigenic component of the vaccine.

The term “vaccine” as used herein, refers to a substance used to stimulate the production of antibodies and/or provide immunity against one or several diseases, prepared from the causative agent of a disease, its products, or a synthetic substitute, treated to act as an antigen without inducing the disease.

A vaccine typically contains an antigen that resembles a disease-causing agent or is made from weakened or killed forms of the disease-causing agent. For example, a vaccine can have one or more antigens from a bacterium, one or more of its surface proteins, or one or more of its membrane components. Vaccines can be prophylactic (to reduce the risk of developing or to ameliorate the effects of a future infection by a natural or “wild” pathogen), or therapeutic (e.g., vaccines against a disease or disorder, which are being investigated). In some embodiments, the vaccine described herein is a prophylactic vaccine. In some embodiments, the vaccine described herein is a therapeutic vaccine. In some embodiments, the vaccine may be both prophylactic and therapeutic.

Initial studies performed with the isolated antigen permitted the production of conjugates that were used to immunise mice and rabbits and generate monoclonal and polyclonal antibodies. The polyclonal antibodies were able to recognise the majority of P. aeruginosa strains in our collection and three monoclonal antibodies were generated one of which was able to recognise and facilitate opsonophagocytic killing of a majority of P. aeruginosa strains. This monoclonal antibody was able to recognise most P. aeruginosa strains in our collection that includes clinical and serotype strains.

Adjuvants may be used to elicit a higher immune response in a subject. As such, adjuvants used according to the present invention may be selected based on their ability to affect antibody titers.

The term “adjuvant” as used herein, refers to any substance that acts to accelerate, prolong, or enhance antigen-specific immune responses when used in combination with specific vaccine antigens. An adjuvant can be a naturally occurring component contained in weakened or killed immunogens. For example, an adjuvant can be a whole cell, a protein, or a protein fragment, or a component of the lipid membrane of a bacterial cell. An adjuvant may also be a synthesized compound. For example, an adjuvant can be an aluminum salt, a phospholipid, or a derivative thereof.

Generally, adjuvanted vaccines can help to elicit stronger local immune reactions as well as systemic immune reactions compared to non-adjuvanted vaccines.

In some examples, the method of raising an immune response further comprises administering an adjuvant.

In some examples, water-in-oil emulsions may be useful as adjuvants. Water-in-oil emulsions may act by forming mobile antigen depots, facilitating slow antigen release and enhancing antigen presentation to immune components. Freund's adjuvant may be used as complete Freund's adjuvant (CFA) which comprises mycobacterial particles that have been dried and inactivated, or as incomplete Freund's adjuvant (IFA), which lacks such particles. Other water-in-oil-based adjuvants may include EMULSIGEN®. EMULSIGEN® comprises micron sized oil droplets that are free from animal-based components and it may be used alone or in combination with other adjuvants, including, but not limited to aluminum hydroxide and CARBIGEN™.

In other examples, immunostimulatory oligonucleotides may also be used as adjuvants. Such adjuvants may include CpG oligodeoxynucleotide (ODN). CpG ODNs are recognized by Toll-like receptor 9 (TLR9), leading to strong immunostimulatory effects. Type C CpG ODNs induce strong IFN-α production from plasmacytoid dendritic cell (pDC) and B cell stimulation as well as IFN-γ production from T-helper (Tx) cells. CpG ODN adjuvant has been shown to significantly enhance pneumococcal polysaccharide (19F and type 6B)-specific IgG2a and IgG3 in mice. CpG ODN also enhances antibody responses to the protein carrier CRM197, particularly CRM197-specific IgG2a and IgG3. Additionally, immunization of aged mice with pneumococcal capsular polysaccharide serotype 14 (PPS14) combined with a CpG-ODN has been shown to restore IgG anti-PPS14 responses to young adult levels. CpG ODNs used according to the present invention may include class A, B or C ODNs. In some embodiments, ODNs may include any of those available commercially, such as ODN-1585, ODN-1668, ODN-1826, ODN-2006, ODN-2007, ODN-2216, ODN-2336, ODN-2395 and/or ODN-M362. In some cases, ODN-2395 may be used. ODN-2395 is a class C CpG ODN that specifically stimulates human as well as mouse TLR9. These ODNs comprise phosphorothioate backbones and CpG palindromic motifs.

In some examples, in preparing a vaccine in accordance with the present disclosure, the glycan is covalently linked, or otherwise conjugated, to an immunogenic carrier molecule. Typically, the immunogenic carrier molecule is a protein or polypeptide.

The term “excipient” or “pharmaceutically acceptable excipient” as used herein refers to any substance combined with a compound and/or composition of the invention before use. In some embodiments, excipients are inactive and used primarily as a carrier, diluent or vehicle for a compound and/or composition of the present invention. An excipient is pharmaceutically if it is physiologically compatible, i.e. it does not produce an adverse or untoward reaction when administered to an animal, including a human or non-human animal as appropriate.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Examples of diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

A vaccine as described herein may preferably prevent, ameliorate and/or treat an infection in a subject caused by P. aeruginosa.

As used herein, the terms “prevent” and “preventing” include the prevention of the recurrence, spread or onset of a P. aeruginosa infection. It is not intended that the present disclosure be limited to complete prevention. In some embodiments, prevention includes delayed onset or reduced severity of infection.

The term “treatment”, “treat”, or “treating” as used herein, refers to obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. Thus, the terms “treat” and “treating” are not limited to the case where the subject (e.g. patient) is cured and the disease is eradicated. Rather, examples of the present disclosure also contemplate treatment that reduces symptoms, and/or delays disease progression.

The term “symptom” of a disease or disorder (e.g., infection with P. aeruginosa) is any morbid phenomenon or departure from the normal in structure, function, or sensation, experienced by a subject and indicative of disease.

The term “amelioration” or “ameliorates” as used herein refers to a decrease, reduction or elimination of a condition, disease, disorder, or phenotype, including an abnormality or symptom.

P. aeruginosa is a significant opportunistic pathogen that causes a variety of life-threatening infections in immunosuppressed or immunocompromised patients. Individuals who are at risk of developing P. aeruginosa infections include cystic fibrosis patients, burn patients, severe neutropenic patients (e.g., cancer patients receiving chemotherapy) and intensive care unit patients receiving respiratory support.

A vaccine as described herein may be administered simultaneously with other existing vaccines.

The vaccines herein may be administered to a subject by any route, including intramuscular, subcutaneous, intradermic, oral, inhalable, intranasal, rectal and intravenous routes. Oral administration may be suitably via a tablet, a capsule or a liquid suspension or emulsion. Alternatively the vaccines may be administered in the form of a fine powder or aerosol via a Dischaler® or Turbohaler®. Intranasal administration may suitably be in the form of a fine powder or aerosol nasal spray or modified Dischaler® or Turbohaler®. Rectal administration may suitably be via a suppository.

The immunoprotective amount of the vaccine may be administered in a single dose or in a series of doses. Where more than one dose is administered, the doses may be administered days, weeks or months apart. In some examples, the vaccine may be administered as a single dose or in a series including one or more boosters.

The dosage of vaccine to be administered a subject and the regime of administration may be determined in accordance with standard techniques well known to those of ordinary skill in the pharmaceutical and veterinary arts, taking into consideration such factors as the intended use, particular antigen, the adjuvant (if present), the age, sex, weight, species, general condition, prior illness and/or treatments, and the route of administration.

In some examples, a therapeutically effective amount of a vaccine or immunogenic composition is used.

A therapeutically effective amount refers to a quantity of a specified agent sufficient to achieve a desired effect in a subject being treated with that agent. For example, this may be the amount of a vaccine useful for eliciting an immune response in a subject and/or for preventing infection. The effective amount of a vaccine (or immunogenic composition) useful for increasing resistance to, preventing, ameliorating, and/or treating infection in a subject will be dependent on, for example, the subject being treated, the manner of administration of the therapeutic composition and other factors.

### Antibodies

In some examples, there is provided a method of producing antibodies specific for a glycan antigen as described herein.

In some examples, antibodies may be developed through immunizing a host with a particular antigen. As discussed above, such an immune response typically leads to the production by the organism of one or more antibodies against the foreign entity, e.g., antigen or a portion of the antigen. In some cases, methods of immunization may be altered based on one or more desired immunization outcomes. As used herein, the term “immunization outcome” refers to one or more desired effects of immunization. Examples include high antibody titers and/or increased antibody specificity for a target of interest. Methods of collecting antibodies are known in the art.

As used herein, the term “antibody” is used in the broadest sense and specifically covers various embodiments including, but not limited to monoclonal antibodies, polyclonal antibodies, multispecific antibodies (e.g. bispecific antibodies formed, for example, from at least two intact antibodies), and antibody fragments such as diabodies so long as they exhibit a desired biological activity. Antibodies are primarily amino-acid based molecules but may also comprise one or more modifications such as with sugar moieties, linkers, detectable labels and the like.

The term “monoclonal antibody” as used herein refers to an antibody obtained from a population of substantially homogeneous cells (or clones), i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen.

In some examples, the antibodies described herein may be humanized.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and capacity.

Methods of the invention are conveniently practiced by providing the compounds and/or compositions used in such methods in the form of a kit. Such a kit preferably contains a composition as described herein. Such a kit preferably contains instructions for the use thereof.

To gain a better understanding of the invention described herein, the following examples are set forth. It should be understood that these examples are for illustrative purposes only. Therefore, they should not limit the scope of this invention in any way.

### Methods and Uses

Generally, there is described herein a method of raising an immune response in a subject, comprising administering an isolated or synthetized glycan, preferably an isolated or synthesized glycan antigen from P. aeruginosa, more preferably an isolated or synthesized glycan antigen from P. aeruginosa linked to a carrier protein in the form of a glycoconjugate to a subject.

In some aspects, there is provided a method of treating a subject having a Pseudomonas aeruginosa infection, suspected of having a Pseudomonas aeruginosa infection, or at risk of developing a Pseudomonas aeruginosa infection, comprising administering an isolated or synthetized glycan, preferably an isolated or synthesized glycan antigen from P. aeruginosa, more preferably an isolated or synthesized glycan antigen from P. aeruginosa linked to a carrier protein in the form of a glycoconjugate to the subject.

In some aspects, there is provided a method of treating a subject having a Pseudomonas aeruginosa infection, suspected of having a Pseudomonas aeruginosa infection, or at risk of developing a Pseudomonas aeruginosa infection, comprising administering an antibody or derivative thereof that is specific for a glycan antigen from P. aeruginosa as identified in this document.

Generally, there is described herein a method of using an antibody such as a monoclonal antibody described herein for diagnosing or treating a P. aeruginosa infection. In one aspect, said antibody is the 1B1 MAb described herein.

In one aspect, the antibody or antigen binding fragment thereof may be used for the treatment of a P. aeruginosa infection.

In one aspect, the antibody or antigen binding fragment thereof may be used in the diagnosis of a P. aeruginosa infection.

In one aspect, a method for treating a P. aeruginosa infection is provided, said method comprising administering the antibody or antigen binding fragment to a subject.

In one aspect, a method for the diagnosis of a P. aeruginosa bacterial infection in an animal is provided, comprising contacting a test sample with the antibody or antigen binding fragment thereof, and detecting specific binding thereto.

### Examples

Pseudomonas aeruginosa produces a variety of cell surface glycans. Previous studies have identified a common polysaccharide (PS) antigen often termed A-band PS that is composed of a neutral D-rhamnan trisaccharide repeating unit as a relatively conserved cell surface carbohydrate. However, the present inventors have discovered a novel epitope not previously identified. Nuclear magnetic resonance (NMR) spectra and chemical analysis of A-PS preparations showed the presence of several additional components. In fact, the carbohydrate antigen consists of an immunogenic methylated rhamnan oligosaccharide at the non-reducing end of the A-band PS. Initial studies performed with the isolated antigen permitted the production of conjugates that were used to immunise mice and rabbits and generate monoclonal and polyclonal antibodies. The polyclonal antibodies were able to recognise the majority of P. aeruginosa strains tested and three monoclonal antibodies were generated one of which was able to recognise and facilitate opsonophagocytic killing of a majority of P. aeruginosa strains. This monoclonal antibody was able to recognise all P. aeruginosa strains in our collection that includes clinical and serotype strains. Synthetic oligosaccharides (mono- to penta-) representing the terminal 3-O-methyl D-rhamnan were prepared and the trisaccharide was identified as the minimum epitope required to effectively mimic the natural antigen recognised by the broadly cross-reactive monoclonal antibody. A conjugate of the synthetic pentasaccharide with a carrier protein raised polyclonal antibodies in mice. These antibodies were also able to recognise whole cells of P. aeruginosa strains tested, emphasising further the ability of the synthetic antigen to effectively mimic the natural antigen. These experiments demonstrate the usefulness of these novel antigens and the corresponding antibodies as a vaccine, therapeutic target, and in a method of treatment of Pa.

TABLE 1
Details of strains used in this study

| Species | Strain | NRCC | Serotype | Details | Source |
|---|---|---|---|---|---|
| P. aeruginosa | PAO1 BAA-47 | 6678 | 5 | wild type | ATCC |
| | PAO1 | 6667 | — | PAO1 (wzy :: Gm) | Lam lab |
| | PAO1 | 6668 | — | PAO1 (wzy : Gm)(Δpa5457) | Lam lab |
| | PAO1 | 6669 | — | PAO1 (wzy :: Gm)(Δpa5458) | Lam lab |
| | PAO1 | 6670 | — | PAO1 (wzy : Gm)(Δpa5459) | Lam lab |
| | 5933 | 6954 | 1 | 33348 | ATCC |
| | 5934 | 6955 | 2 | 33349 | ATCC |
| | 5939 | 6956 | 6 | 33354 | ATCC |
| | 5943 | 6957 | 10 | 33357 | ATCC |
| | 5944 | 6958 | 11 | 33358 | ATCC |
| | 170003 | 6959 | 16 | 33363 | ATCC |
| | 5937 | 6960 | 5 | 33352 | ATCC |
| | 001S4-1 | 6944 | — | Clinical isolate | Sad lab |
| | 003S-20 | 6945 | — | Clinical isolate | Sad lab |
| | 003E-9 | 6946 | — | Clinical isolate | Sad lab |
| | 004S-3 | 6947 | — | Clinical isolate | Sad lab |
| | 004E-8 | 6948 | — | Clinical isolate | Sad lab |
| | 006S3-1 | 6949 | — | Clinical isolate | Sad lab |
| | 006S4-1 | 6950 | — | Clinical isolate | Sad lab |
| | 009S-7 | 6951 | — | Clinical isolate | Sad lab |
| | 014S-1 | 6952 | — | Clinical isolate | Sad lab |
| | 014E-31 | 6953 | — | Clinical isolate | Sad lab |
| M. catarrhalis | lgt2/lgt4 | 6541 | — | Negative control | Cox lab |
| N. meningitidis | 8047 lpt3 | 6263 | — | Negative control | Cox lab |

### Example 1A: Isolation and Oxidation of A-PS

The following methods were used to isolate the A-band terminal epitope. In addition to isolating it via the LPS, it was isolated directly from cells. The yield of A-PS was higher from EDTA extraction, compared to its isolation from LPS, but purification was more challenging when EDTA extraction was used.

The A-band PS was isolated from the PAO1 Wzy::Gm (mutant lacking band B OPS) LPS by acetic acid hydrolysis with subsequent size-exclusion chromatography. PS fraction was then purified by anion-exchange or reverse-phase HPLC. The yield was usually low, about 5 mg of A-PS from 100 mg of the LPS, and did not scale up proportionally with a higher amount of starting LPS.

Alternatively A-PS was extracted from the same mutant cells with EDTA. This method has been previously reported as an LPS extraction method, but no significant amount of the LPS was extracted in this way. Three main components were found in the preparation: A-PS, psl-polysaccharide with the structure described previously [15], and a cyclic phosphorylated glucan [16]. Cyclic glucan signals were not well visible in the spectra of the whole mixture, but it was obtained in pure form after anion-exchange separation in the fractions eluted at high salt concentration, after A-band and psl polysaccharides. NMR spectra of the isolated compounds showed no signals attributable to the lipids or other components of the LPS. Thus at least some part of band-A rhamnan was not linked to the LPS as suggested previously [17]. The yield of the A-PS was much higher from the EDTA extraction, compared to its isolation from the LPS, but purification was more challenging.

The A-PS was purified on reverse-phase HPLC column or Sep-Pak C18 cartridge, where it was fully retained in water and could be eluted with 30% methanol. All components of the A-PS, including those producing minor anomeric signals and methyl signals in NMR spectra elute together. In column C18 chromatography, with water-methanol gradient elution, A-PS was eluted in wide area of methanol gradient starting at about 20% MeOH, without visible peaks.

A-PS contained acidic components and was partially retained on the anion-exchange column, eluting in NaCl gradient in several fractions. This behaviour may be indicative of the linkage of the A-PS to the LPS core, which is acidic due to phosphorylation.

Growth of Pa strains: P. aeruginosa strain PAO1 (wzy::Gm) (NRCC 6667) known to lack B-band LPS was grown in order to isolate the A-band polysaccharide (PS). One Difco Brain Heart Infusion (BHI) agar plate with 100 μg mL⁻¹ gentamicin sulfate streaked from frozen stock was incubated at 37° C. in a Forma incubator. After 7 h incubation all plate growth material was suspended in 1.5 mL BHI broth and vortexed well to suspend material. Ten BHI agar plates with 100 mg L⁻¹ gentamicin sulphate were equally inoculated with the suspended material and spread using the hockey stick method and incubated at 37° C. Two 1 L/4 L baffle flasks with BHI broth with 100 mg L⁻¹ gentamicin sulfate were inoculated with the proceeds from the 10 plates following 16 h incubation. Flasks were incubated in a Forma shaker incubator at 37° C. and 200 RPM for 2 h. Once A_{600 nm} reached an optical density of 2 these flask cultures were used to inoculate 22 L of BHI broth with 100 mg L⁻¹ gentamicin sulphate in a 30 L new MBR (Multiple Bioreactors) fermenter. Dissolved oxygen was controlled at 15% saturation with variable air flow, agitation rate and with oxygen blending as required. After 8¾ hours growth, temperature was decreased to 10° C. until harvesting. After 22 h the A_{600 nm} had reached 13.4, with a pH of 8.35. The culture was concentrated to 4 L using a Millipore tangential flow Pellicon harvesting unit (3×0.22μ membranes). The 4 L of concentrated cells were killed with addition of 90 ml of a 95% (w/v) phenol solution and placed in Forma shaker incubator at 10° C. and shaken at 175 RPM for 4 h. A viability check of material following washing 3× with PBS was performed. The 4 L concentrate was spun in a Sorval RC6+ centrifuge at 11K RPM for 30 minutes to pellet cells. 2767 g wet wt of a gelatinous sloppy pellet was obtained and dispersed into three 1 L containers. One container was spun for an additional 30 minutes at 11K RPM resulting in a final wet weight of 1110 g. Cells were freeze dried and stored for future use.

Several other strains as detailed in Table 1 were also grown and used in this study. All biomass was harvested from a 24 L fermenter following the inoculation build up and growth described above.

Isolation of A-PS by EDTA extraction of whole cells: 20 g dry cells of P. aeruginosa strain PAO1 (wzy::Gm) (NRCC 6667) were stirred in 10% (w/v) disodium EDTA for 1 h at 25° C. The solution was dialyzed and acetic acid was added to 10% final concentration to precipitate nucleic acids. The precipitate was removed by centrifugation, and the solution dialyzed and freeze dried. The material was dissolved in water, ultra-centrifuged, and the resulting clear solution was separated on a Biogel P6 column. A polymeric fraction was collected and separated by anion-exchange chromatography.

Isolation of A-PS from LPS: LPS was isolated as described previously [14] from P. aeruginosa cells from strain PAO1 (wzy::Gm) (NRCC 6667). LPS (100 mg) was hydrolyzed with 2% acetic acid (100° C., 2 h). The resulting solution was centrifuged to remove precipitate and separated on a Biogel P6 column. A polymeric fraction was collected and separated by anion-exchange chromatography.

Periodate oxidation: The polymeric fractions from both of the above isolations (10 mg at a time) were dissolved in water (2 mL) and NaIO₄ (20 mg) was added. The solution was kept at RT in the dark for 24 h, after which ethylene glycol (0.2 mL) and an excess of NaBD₄ were added and the solution was maintained for an additional 1 h, neutralised with 0.2 mL of AcOH and desalted on Sephadex G-15 column. Product was hydrolyzed with 2% AcOH for 2 h at 100° C., separated on Sephadex G-15 column to give OS1. A similar methodology was followed for production of OS2 from OS1, whereby OS1 was oxidized with periodate, reaction quenched with ethylene glycol without borohydride reduction and purified on a Sephadex G-15 column. The structures of OS1 and OS2 are shown in FIG. 1.

Gel chromatography: Gel chromatography was performed on Sephadex G-15 column (1.5×60 cm) or Biogel P6 column (2.5×60 cm) in 1% acetic acid, monitored by refractive index detector (Gilson).

Anion-exchange chromatography: Sample up to 50 mg was injected into HiTrap Q column (Amersham, two columns by 5 mL each connected together) in water at 3 mL min⁻¹, washed with water for 5 min, then eluted with a linear gradient from water to 1 M NaCl over 1 h with UV detection at 220 nm and spot test on silica TLC plate with development by dipping in 5% H₂SO₄ in ethanol and heating with heat gun until brown spots become visible. Carbohydrate positive samples were pooled and desalted on a Sephadex G-15 column in 1% AcOH with refractive index detector.

### Example 1B: Structural Characterization of an Immunodominant Epitope a the Tip of the A-Band Rhamnan of Pseudomonas aeruginosa (Pa)

Determination of neutral and amino sugars as alditol acetates: Samples (0.2-1 mg) were hydrolyzed with 3 M TFA (120° C., 3 h), dried, reduced with NaBD₄, and excess reagent destroyed with 0.5 mL of AcOH. The solution was dried under a stream of air, dried twice following addition of MeOH (1 mL), and acetylated with 0.2 mL Ac₂O with 0.2 mL pyridine for 30 min at 100° C. The reaction mixture was dried, analyzed by GC-MS on Thermo Trace 1310 instrument with ITQ1100 ion trap detector, capillary column HP-5, 160-260° C. by 4° C. min⁻¹.

Absolute configurations: To the samples and prepared standards (˜0.5 mg) (R)- or (RS)-2-octanol (0.2 mL) and acetyl chloride (20 μL) were added at RT, heated in closed vials (100° C., 2 h). The reaction mixtures were dried by air stream, acetylated (0.2 mL Ac₂O with 0.2 mL pyridine, 100° C., 30 min), and dried and then analyzed by GC-MS as described above.

NMR spectroscopy: NMR experiments were carried out on a Bruker AVANCE III 600 MHz (¹H) spectrometer with 5 mm Z-gradient probe with acetone internal reference (2.225 ppm for ¹H and 31.45 ppm for ¹³C) using standard pulse sequences cosygpprqf (gCOSY), mlevphpr (TOCSY, mixing time 120 ms), roesyphpr (ROESY, mixing time 500 ms), hsqcedetgp (HSQC), hsqcetgpml (HSQC-TOCSY, 80 ms TOCSY delay) and hmbcgplpndqf (HMBC, 100 ms long range transfer delay). Resolution was kept <3 Hz/pt in F2 in proton-proton correlations and <5 Hz/pt in F2 of H—C correlations. The spectra were processed and analyzed using the Bruker Topspin 2.1 program.

Monosaccharides were identified by COSY, TOCSY and NOESY cross peak patterns and ¹³C NMR chemical shifts. Amino group location was concluded from high field signal position of aminated carbons (CH at 45-60 ppm). Connections between monosaccharides were determined from transglycosidic NOE and HMBC correlations.

Mass spectrometry: ESI MS was obtained using Waters SQ Detector 2 instrument. Samples were injected in 50% MeCN with 0.1% TFA.

Structure: Pseudomonas aeruginosa A-PS contains a main component which is composed of D-rhamnose trisaccharide repeating units:

| -2-α-Rha-3-α-Rha-3-α-Rha- | | | A-PS repeating unit |
|---|---|---|---|
| A | B | C | |

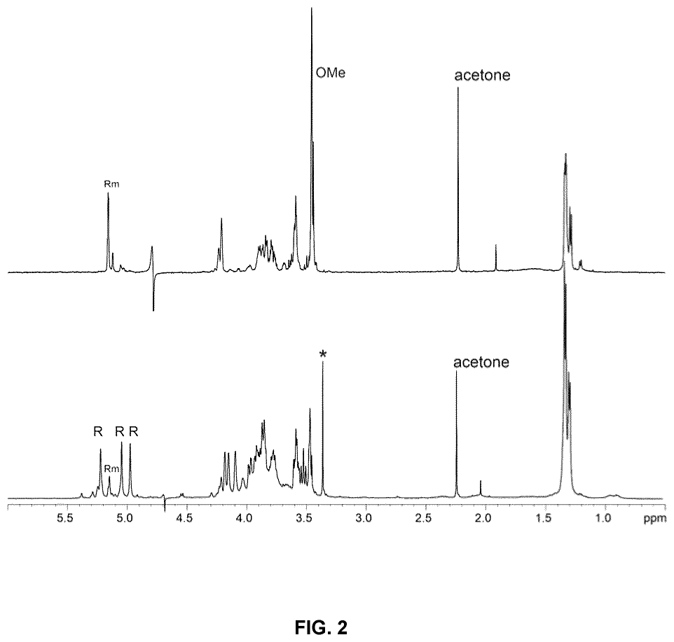

NMR spectra of the A-PS preparations contained signals of the repeating units and additional signals (FIG. 2). Some of the minor anomeric signals were previously identified as belonging to 3-O-methyl-rhamnose [18], which has signals of O-methyl groups around 3.3 ppm, but the complete structure was never determined.

Polysaccharides obtained from LPS and by EDTA extraction had similar NMR spectra. There were minor differences, but it was not possible to identify these minor components and distinguish impurities from real constituents of the A-PS, due to the complex nature of the spectra. Since A-PS is presumptively linked to the LPS core, it was anticipated that A-PS obtained from the LPS should contain core components, but these were not reliably identified.

Monosaccharide analysis of A-PS (GC-MS of alditol acetates) showed the presence of Rha as a main component, with small quantity of Man, Glc, 2-O-methyl-Rha, 3-O-methyl-Rha (about 5-8 times more than other minor components), 2-O-methyl-Man, and 3-O-methyl-Man. No heptose derivatives were detected.

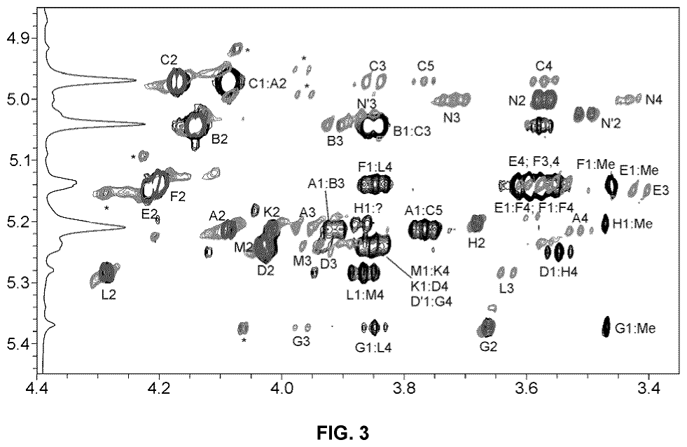

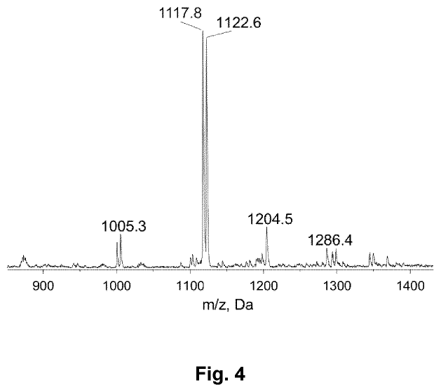

Periodate oxidation of the A-PS with subsequent NaBD₄ reduction, mild hydrolysis, and separation of the products on Sephadex G-15 column led to the isolation of the expected D-rhamnan oxidation product α-D-Rha-3-α-D-Rha-2-Grold and an oligosaccharide OS1 (FIG. 1). OS1 was analyzed by NMR and ESI-MS. Interpretation of the 2D NMR spectra of OS1 (Table 2, FIG. 3) led to the identification of the terminal 3-O-methyl-α-Rha residue (E) and several overlapping spin-systems of a homooligomer of 4-substituted 3-O-methyl-α-Rha residues (F). The number of the residues in this oligomer was not obvious from NMR data, but MS established that there were five 3-O-methyl-Rha residues (FIG. 4). The next residue in the sugar chain of OS1 after the 3-OMe rhamnan oligosaccharide was represented by 3-O-methyl-α-Man (L), which in turn was linked to O-2 of 1-deuterated erythritol X. 3-O-methyl-Man was identified by GC-MS (alditol acetates) using K. pneumoniae O5 OPS as a source of a standard [19]. Erythritol could only be produced by oxidation-reduction of 4-substituted hexopyranose. The position of methyl groups was deduced from NOE and HMBC data and large downfield shift of ¹³C signals at the methylation positions.

TABLE 2
NMR data for OS1 and free 3-O-methyl-rhamnose,
isolated from A-PS (δ, ppm, 500 MHz 25°
C.). Me in free Rha3Me: 3.44/57.3 ppm.

| | H/C-1 | H/C-2 | H/C-3 | H/C-4 | H/C-5 | H/C-6 |
|---|---|---|---|---|---|---|
| Rha3OMe E, 1 | 5.16 | 4.23 | 3.43 | 3.50 | 3.80 | 1.29 |
| | 102.4 | 67.3 | 80.6 | 71.8 | 70.6 | 17.7 |
| Rha3OMe F, 1 | 5.15 | 4.21 | 3.58 | 3.59 | 3.89 | 1.33 |
| | 102.4 | 67.4 | 81.7 | 79.1 | 69.0 | 18.3 |
| Man3OMe L, 1 | 5.12 | 4.24 | 3.67 | 3.83 | 3.83 | 3.78; |
| | 99.8 | 67.3 | 81.9 | 73.8 | 72.9 | 67.1 |
| tetritol X, 1 | 3.75; | 3.79 | 3.90 | 3.63; | | |
| | 59.7 | 78.5 | 72.3 | 63.7 | | |
| free α-Rha3Me | 5.15 | 4.17 | 3.48 | 3.47 | 3.88 | 1.27 |
| | 95.1 | 67.6 | 80.6 | 72.3 | 69.4 | 18.1 |
| free β-Rha3Me | 4.85 | 4.20 | 3.31 | 3.39 | 3.41 | 1.29 |
| | 94.8 | 68.1 | 83.0 | 72.0 | 73.2 | 18.1 |

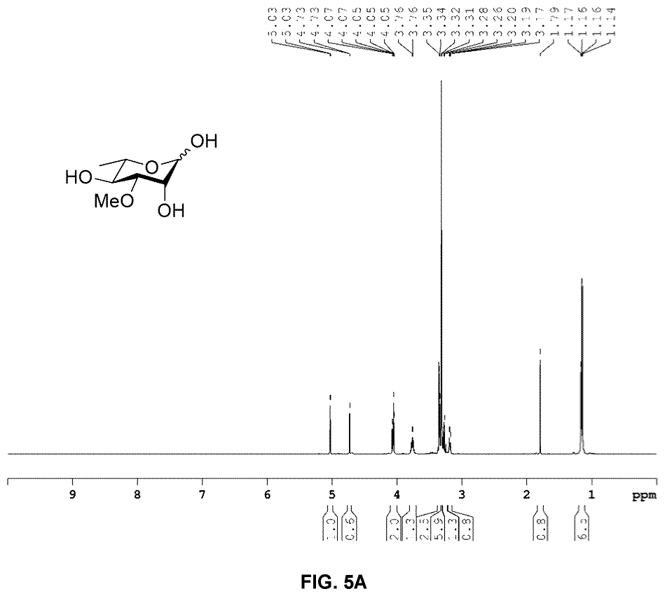

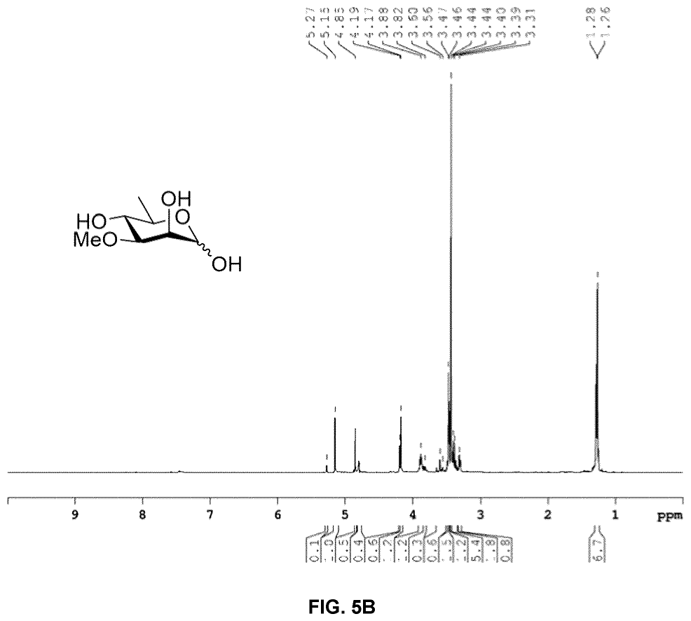

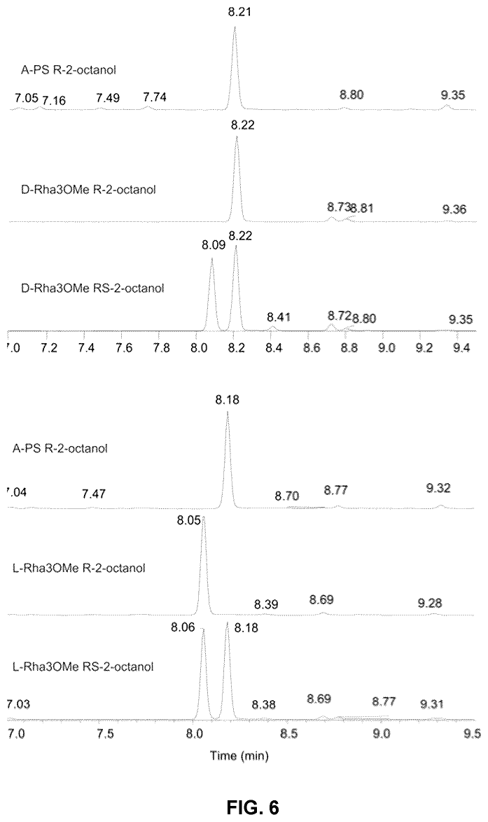

In order to enable the unequivocal determination of the absolute configuration of the 3-OMe rhamnose, as both isomers of rhamnose have been identified in P. aeruginosa LPS [20], synthetic standards of the D- and L-rhamnose monosaccharides were prepared. NMR analysis confirmed the authenticity of these standards (FIGS. 5A and 5B). Absolute D-configuration of 3-O-methyl-Rha was determined by GC-MS of acetylated 2-octanol derivatives of OS1 compared to the same derivatives obtained from synthetic standards (FIG. 6).

Positive mode ESI MS of compound OS1 showed two peaks at m/z 1117.8 and 1122.6 in 1:1 ratio, corresponding to ammonium and sodium adducts of Rha₅Man₁Tetritol-1d₁Me₆, calculated exact mass 1099.5 (FIG. 4). Thus OS1 contains five residues of 3-O-methyl-D-Rha connected by 1-4-linkages.

A similar oligosaccharide was suggested previously [18], but xylose was detected in that study instead of 3-O-methyl-α-Man and linkage positions were not identified.

Interpretation of the NMR spectra of the intact A-PS confirmed the signals of the repeating rhamnan trisaccharide -A-B-C- (data not shown), and also showed the presence of less intense spin systems corresponding to the full OS1, and two residues G and H, identified as 2-O-methyl-Man (G) and 2-O-Me-Rha (H) (Table 3). Spectra always contained two weak spin systems of terminal α-Glc (N, N′), assumed to be Glc from Pseudomonas LPS core, but these residues produced no interpretable NOEs.

TABLE 3
NMR data for A-PS ((δ, ppm, 600 MHz, 40° C.). Methyl
groups: G, H: 3.47/59.7 ppm; E, F, L: 3.46/57.5 ppm.

| | H/C-1 | H/C-2 | H/C-3 | H/C-4 | H/C-5 | H/C-6 |
|---|---|---|---|---|---|---|
| Rha A | 5.21 | 4.09 | 3.97 | 3.52 | 3.85 | 1.32 |
| | 102.1 | 79.4 | 71.2 | 73.6 | 70.5 | 18.0 |
| Rha B | 5.05 | 4.15 | 3.91 | 3.58 | 3.89 | 1.32 |
| | 103.5 | 71.2 | 79.1 | 72.8 | 70.5 | 18.0 |
| Rha C | 4.97 | 4.17 | 3.85 | 3.58 | 3.77 | 1.29 |
| | 103.3 | 71.2 | 79.3 | 72.8 | 70.7 | 18.0 |
| Rha3OMe E | 5.15 | 4.22 | 3.42 | 3.50 | 3.82 | 1.29 |
| | 102.5 | 67.5 | 80.8 | 72.0 | 69.1 | 18.0 |
| Rha3OMe F | 5.13 | 4.20 | 3.55 | 3.58 | 3.86 | 1.33 |
| | 102.5 | 67.6 | 81.9 | 79.3 | 69.0 | 18.0 |
| Man3OMe L | 5.27 | 4.28 | 3.62 | 3.85 | 3.78 | 3.78; |
| | 102.5 | 67.4 | 81.8 | 73.9 | 73.3 | 62.2 |
| Man M | 5.23 | 4.03 | 3.94 | 3.85 | 3.74 | 3.78; |
| | 102.6 | 71.9 | 72.1 | 75.4 | 73.5 | 62.2 |
| Man K | 5.21 | 4.01 | 3.94 | 3.85 | 3.74 | 3.78; |
| | 102.5 | 71.9 | 72.1 | 75.4 | 73.5 | 62.2 |
| Man D | 5.25 | 4.02 | 3.94 | 3.85 | 3.74 | 3.78; |
| | 102.5 | 71.9 | 72.1 | 75.4 | 73.5 | 62.2 |
| Man2OMe G | 5.37 | 3.66 | 3.96 | 3.76 | | |
| | 99.4 | 83.1 | 71.2 | 75.6 | | |
| Rha2OMe H | 5.20 | 3.68 | 4.02 | 3.55 | 3.83 | 1.33 |
| | 102.1 | 83.1 | 71.9 | 80.7 | | |
| α-Glc N | 5.00 | 3.57 | 3.71 | 3.42 | 3.76 | 3.87 |
| | 99.2 | 72.7 | 74.5 | 70.8 | | |
| α-Glc N′ | 5.03 | | | | | |
| | 100.3 | | | | | |

3-O-methyl-Man L in the A-PS was linked to O-4 of α-Man M, thus erythritol X in OS 1 originated from the oxidized Man M. Several spin-systems of α-Man were present and they forma trisaccharide -4-α-Man-4-α-Man-4-α-Man- (M-K-D), linked either to 2-O-methyl-Rha H or 2-O-methyl-Man G. Further tracing of the chain was not possible, because no other components could be identified. Connection between D-rhamnan trisaccharide repeating units and methylated oligosaccharide was not identified.

Since all signals of the OS1 remained at the same position in the A-PS and OS1, we can conclude that oligomer of 3-O-methyl-Rha occupied the non-reducing end of the A-PS chain and by virtue of its terminal location may be immunogenic.

As described herein, the detailed structure for the non-reducing end of the A-band PS has been determined and it has been shown that, rather than containing merely the neutral rhamnan trisaccharide repeat unit, this polymer is capped with a pentasaccharide of 3-O-methyl rhamnose residues linked via a methyl mannose molecule to a neutral mannose trisaccharide. The linkage point to the neutral rhamnan has not been established, however the fact that identical signals for the 3-O-methyl Rha residues remain at the same position in A-PS and OS1 strongly suggests that the oligomer of 3-O-methyl-Rha occupies the non-reducing end of the A-PS chain. Others have observed signals consistent with 3-O-methyl rhamnose residues in their A-PS preparations, but no one has been able to elucidate the structural unit described here [18, 21].

### Example 2: Production and Immunological Evaluation of Glycoconjugates (BSA-OS2 and CRM-OS2) Made with Isolated A-Band Epitope (OS2)

Three mAbs have been identified that are specific for the A-PS terminus, and preliminary epitope mapping studies suggest that they recognise different regions within this terminal unit. These mAbs, and to a lesser extent the polyclonal sera raised to the A-PS terminus, were able to facilitate killing of P. aeruginosa strains in SBA and OPA experiments. Previous studies by Makarenko et al utilising a synthetic glycoconjugate vaccine based upon the neutral rhamnan had not been able to raise antibodies that were able to recognise, let alone facilitate killing of P. aeruginosa strains [25]. In addition, studies from the laboratory of Lam had observed that a mAb N1F10, which is specific for the neutral rhamnan, was able to recognise approximately 70% of P. aeruginosa strains [26], but recent studies with N1F10, or scFv derivatives thereof, did not show any functionality [27]. It appears that a key region of this conserved antigen may have been overlooked in previous studies, whereby the examples described herein demonstrate that the methylated tip region of the A-PS is indeed immunogenic and the immune response is able to facilitate opsonophagocytic activity. One of the three mAbs described herein, 1B1, appears to recognise a highly conserved epitope, as the majority of P. aeruginosa strains tested were recognised by it, including commonly encountered virulent serotypes and recent clinical isolates. The other two mAbs described herein, 3B8 and 3C4, are more specific than the 1B1 mAb, with their recognition restricted to the wt serotype 5 strains PAO1 BAA-47 and 5937. Studies with synthetic oligosaccharides based upon the terminal methylated pentasaccharide confirmed the broader specificity of mAb 1B1 when compared to the other mAbs described herein. The inhibition ELISA data with synthetic oligosaccharides revealed that mAb 1B1 could be effectively inhibited with a 3-O-methyl rhamnan trisaccharide. Given the broad cross-reactivity of mAb 1B1, recognising the majority of clinical isolates in our collection and the ATCC serotype strains responsible for the majority of the disease caused by P. aeruginosa, it appears that the synthetic oligosaccharides described herein have promise as vaccine antigens or as a means to generate broadly cross reactive therapeutics.

The importance of the A-PS antigen remains a topic of some discussion in the field, but the observation that in the CF lung, the serotype specific O-antigen is no longer established, but the A-PS remains, suggests that the A-PS immunogenic tip structure could be a key epitope to target in a clinical niche.

Conjugates: BSA and CRM conjugates were prepared via standard direct reductive amination chemistry as described previously [22]. Briefly, the oxidised A-band terminal epitope material (OS2) (4.5 mg and 2 mg) was reacted with CRM (2.3 mg) or BSA (1 mg) respectively, by mixing solutions of the two reactants in water and left at RT before lyophilising. The lyophilised material was dissolved in 10 mL of 100 mM NaPO₄, then NaCNBH₃ (5 mg mL⁻¹) was added and the resulting solutions were left at room temperature (RT) for 16 h. The conjugate was purified on a 30 KDa molecular weight cut off spin column in PBS with 10 mM sodium citrate and the supernatant was assayed for protein and filter sterilized and frozen at −20° C. An aliquot was examined by MALDI-MS as described previously [23].

Generation of anti-P. aeruginosa A-band terminal epitope antibodies: To produce antibodies that target P. aeruginosa A-band terminal epitopes, six Balb/C mice (6-8 weeks) and two New Zealand white rabbits (1.5-2 kg) were immunized with glycoconjugates of the purified and oxidised terminal A-band molecule with CRM as the carrier protein. Immunisations were scheduled and adjuvanted as described previously [22].

mAb production: Mice immunised for polyclonal sera were screened for recognition of the A-band PS and the mouse giving the highest titer was selected for the fusion following a final i.v. injection. All manipulations for the fusion of the harvested spleen cells from the immunised mice, the hybridoma selection and growth and purification of high density mAb supernatant were performed as described previously [24].

Characterisation of Anti-P. aeruginosa A-Band Terminal Epitope mAbs

Antibody variable region sequencing: The DNA sequences encoding the rearranged variable heavy chain (V_{H}) and variable light chain (V_{L}) domains of the three mAbs generated (1B1, 3C4 and 3B8) were determined by Illumina MiSeq amplicon sequencing as described previously [24]. Amino acid sequences for the VH and VL domains of the three mAbs are provided in Table 4.

TABLE 4
Antibody sequences

| SEQ | | |
|---|---|---|
| ID NO: | Sequence | Description |
| 1 | GFPFSDYY | 1B1 VH CDR1 |
| 2 | INGDGGTT | 1B1 VH CDR2 |
| 3 | VRGRRITGTGAIDY | 1B1 VH CDR3 |
| 4 | DVKLVESGGGLVKVGGSLKISCAASGFPFSDYYMAWVG | 1B1 VH |
| | QTPEKRLELVAAINGDGGTTYYPDTVKGRFIISRDNAKNT | |
| | LYLQMSSLRSEDTALYYCVRGRRITGTGAIDYWGQGTS | |
| | VTVSS | |
| 5 | gacgtgaagctcgtggagtctgggggaggcttagtgaaggttggagggtccctaaaaattt | 1B1 VH coding |
| | catgtgcagcctctggattccccttcagtgactattatatggcttgggttggccagactccaga | sequence |
| | gaagaggctggagttggtcgcagccattaatggtgatggtggtaccacctactatccagaca | |
| | ctgtgaagggccgattcatcatttccagagacaatgccaagaacaccctgtacctgcaaatg | |
| | agcagtctgaggtctgaggatacagccttgtattattgtgtaagagggagaaggataactgg | |
| | aactggggctatagactactggggtcaaggaacctcagtcaccgtctcctca | |
| 6 | SNISY | 1B1 VL CDR1 |
| — | GTS | 1B1 VL CDR2 |
| 7 | QIYHSYPYT | 1B1 VL CDR3 |
| 8 | QIFLTQSPVILSASPGEKVTMTCSATSNISYMYRYQQKPGS | 1B1 VL |
| | SPKPWIYGTSNLASRVPARFSGSGSGTSYSLTISNMEAEDA | |
| | ATYYCQIYHSYPYTFGGGTKLEIK | |
| 9 | caaatttttctcacccagtctccagttatcctgtctgcatctccaggggagaaggtcaccatga | 1B1 VL coding |
| | cctgcagtgccacctcaaatataagttacatgtacaggtaccagcagaagccaggatcctca | sequence |
| | cccaaaccctggatttatggcacatccaacctggcttctcgagtccctgctcgcttcagtggc | |
| | agtggatctgggacctcttattctctcacaatcagcaacatggaggctgaagatgctgccact | |
| | tattactgccagatatatcatagttacccgtacacgttcggaggggggaccaagctggaaat | |
| | aaaa | |
| 10 | GYSFTDYN | 3B8 VH CDR1 |
| 11 | IDPYNGGT | 3B8 VH CDR2 |
| 12 | AKGGYRYDAWFAY | 3B8 VH CDR3 |
| 13 | EIQLQQSGPELVKPGASVKVSCKASGYSFTDYNMYWVK | 3B8 VH |
| | QSHGKSLEWVGYIDPYNGGTTYNQKFKGKATLTVDKSSS | |
| | TAFMHLNSLTSEDSAVYYCAKGGYRYDAWFAYWGQGT | |
| | LVTVSA | |
| 14 | gagatccagctgcagcagtctggacctgagctggtgaagcctggggcttcagtgaaggtat | 3B8 VH coding |
| | cctgcaaggcttctggttactcattcactgactacaacatgtactgggtgaagcagagccatg | sequence |
| | gaaagagccttgagtgggttggatatattgatccttacaatggtggtactacctacaaccaga | |
| | agttcaagggcaaggccacattgactgttgacaagtcctccagcacagccttcatgcatctc | |
| | aacagcctgacatctgaggactctgcagtctattactgtgcaaagggcggctataggtacga | |
| | cgcctggtttgcttactggggccaagggactctggtcactgtctctgca | |
| 15 | QSLENNNGNTY | 3B8 VL CDR1 |
| — | RVS | 3B8 VL CDR2 |
| 16 | LQVTHVPWT | 3B8 VL CDR3 |
| 17 | DAVMTQTPLSLPVSLGDHASISCRSSQSLENNNGNTYLN | 3B8 VL |
| | WYLQKPGQSPQLLIYRVSNRFSGVLDRFSGSGSGTDFTLKI | |
| | SRVEAEDLGVYFCLQVTHVPWTFGGGTKLEIK | |
| 18 | gatgctgtgatgacccaaactccactctccctgcctgtcagtcttggagatcacgcctccatct | 3B8 VL coding |
| | cttgcaggtctagtcagagccttgaaaacaataatggaaacacctatttgaactggtacctcc | sequence |
| | agaaaccaggccagtctccacagctcctgatctacagggtttccaaccgattttctggggtcc | |
| | tagacaggttcagtggtagtggatcagggacagatttcacactgaaaatcagcagagtgga | |
| | ggctgaggatttgggagtttatttctgcctccaagttacacatgtcccgtggacgttcggtgga | |
| | ggcaccaagctggaaatcaaa | |
| 19 | GFTFTDYY | 3C4 VH CDR1 |
| 20 | IRNKANGYTT | 3C4 VH CDR2 |
| 21 | TRAIYYGYDGFAY | 3C4 VH CDR3 |
| 22 | EVKLVESGGGLVQPGGSLRLSCATSGFTFTDYYMSWVRQ | 3C4 VH |
| | PPGKALEWLGFIRNKANGYTTEYSASVKGRFTISRDNSQS | |
| | ILYLQMNTLRAEDSATYYCTRAIYYGYDGFAYWGQGTL | |
| | VTVSA | |
| 23 | gaggtgaagctggtggagtctggaggaggcttggtacagcctgggggttctctgagactct | 3C4 VH coding |
| | cctgtgcaacttctgggttcaccttcactgattactacatgagctgggtccgccagcctccag | sequence |
| | gaaaggcacttgagtggttgggttttattagaaacaaagctaatggttacacaacagagtaca | |
| | gtgcatctgtgaagggtcggttcaccatctccagagataattcccaaagcatcctctatcttca | |
| | aatgaacaccctgagagctgaggacagtgccacttattactgtacaagggccatctactatg | |
| | gttacgacgggtttgcttactggggccaagggactctggtcactgtctctgca | |
| 24 | QSLENSNGNTY | 3C4 VL CDR1 |
| — | RVS | 3C4 VL CDR2 |
| 25 | LQVTHVPFT | 3C4 VL CDR3 |
| 26 | DAVMTQTPLSLPVSLGDQASISCRSSQSLENSNGNTYLN | 3C4 VL |
| | WYLQKPGQSPQLLIYRVSNRFSGVLDRFSGSGSGTDFTLKI | |
| | SRVEAEDLGVYFCLQVTHVPFTFGSGTKLEIK | |
| 27 | gatgctgtgatgacccaaactccactctccctgcctgtcagtcttggagatcaagcctccatct | 3C4 VL coding |
| | cttgcaggtctagtcagagccttgaaaacagtaatggaaacacctatttgaactggtacctcc | sequence |
| | agaaaccaggccagtctccacagctcctgatctacagggtttccaaccgattttctggggtcc | |
| | tagacaggttcagtggtagtggatcagggacagatttcacactgaaaatcagcagagtgga | |
| | ggctgaggatttgggagtttatttctgcctccaagttacacatgtcccattcacgttcggctcgg | |
| | ggacaaagttggaaataaaa | |

*Bold highlighting indicates the location of CDR sequences

ELISA: The binding of anti-P. aeruginosa A-band terminal epitope mAbs and polyclonal sera to BSA conjugates, purified LPS and to whole cells was evaluated by ELISA as described previously [24].

mAb competition ELISA: Wells of Nunc Maxisorp EIA plates were coated with 1 μg of P. aeruginosa LPS in PBS overnight at 4° C. and then brought to RT before use. Plates were blocked with 1% BSA-PBS for 1 h at RT, and then wells were washed with PBS-T. One mAb (1B1 or 3B8) was titrated and added to the plate for 1 h at RT. Following washing with PBS-T, the second mAb (3C4) was added at a pre-determined concentration and allowed to incubate for 1 h at RT. Following washing with PBS-T, AP-labeled goat anti-mouse IgG2b (Southern Biotech) specific for mAb 3C4, diluted 1:250 in 1% BSA-PBS was added for 1 h at RT. The plates were then washed with PBS-T and developed with Phosphatase Substrate System (Kirkegaard and Perry Laboratories). After 60 min absorbance was measured at 405 nm using a microtiter plate reader.

mAb inhibition ELISA: Wells of Nunc Maxisorp EIA plates were coated with either 1 μg of P. aeruginosa LPS in PBS overnight at 4° C. or P. aeruginosa killed whole cells in dH₂O in a drying oven overnight, and then brought to RT before use. Plates were blocked with 1% BSA-PBS for 1 h at RT, and then wells were washed with PBS-T. During the blocking step, the inhibition was set-up. Either LPS or a synthetic oligosaccharide was added to a tube at either 1 mg/ml or 3 mg/ml and then a serial dilution with 1% BSA-PBS was performed, before an equal volume of mAb (1B1, 3B8 or 3C4) was added at a constant concentration of 10 μg/ml in 1% BSA-PBS, this mixture was incubated for 1 h at RT. Following washing of the EIA plate with PBS-T, 100 μl from the mixed tubes was added to the plate and allowed to incubate for 1 h at RT for LPS coated plates, or 3 h at RT for killed whole cell coated plates. Following washing with PBS-T, the appropriate AP-labeled goat anti-mouse Ig (Southern Biotech) specific for the mAb used, diluted 1:250 in 1% BSA-PBS, was added for 1 h at RT. The plates were then washed with PBS-T and developed with Phosphatase Substrate System (Kirkegaard and Perry Laboratories). After 60 min, absorbance was measured at 405 nm using a microtiter plate reader.

Serum bactericidal assay: The ability of the polyclonal sera and mAbs to facilitate bactericidal killing of selected P. aeruginosa strains was determined as described previously [22].

Opsonophagocytic assay: The ability of the polyclonal sera and mAbs to facilitate opsonophagocytic killing of selected P. aeruginosa strains was determined as described previously [23].

Surface plasmon resonance (SPR): SPR binding assays were performed at 25° C. on a Biacore T200 instrument in HBS-EP running buffer (Cytiva Life Sciences, Mississauga, Canada), essentially as described previously [24]. Briefly, approximately 13000 RUs of IgM (PA 1B1 and control Fn 4F1) were amine coupled to a CM7S sensor chip in 10 mM acetate buffer, pH 4.0 (Cytiva). Synthetic oligosaccharides were reconstituted in HBS-EP to 25 mM and a series of dilutions prepared for injection over IgM surfaces. The contact time was 60 s and the dissociation time 120 s (mono-, di-) or 180 s (tri-, tetra-, penta-). The following concentration ranges were injected: mono- (1 mM-62.5 μM), di- (200 μM-12.5 μM) and tri-, tetra- and penta- (10 μM-625 nM). Mcat lgt2/4 an oligosaccharide from Moraxella catarrhalis, served as a control and was injected at 10 μM. Surfaces were regenerated by extensive washing with HBS-EP. Reference flow cell subtracted sensorgrams were fit to a 1:1 binding model to determine binding kinetics and affinities, or affinities were determined by steady-state analysis.

### Example 2: Results

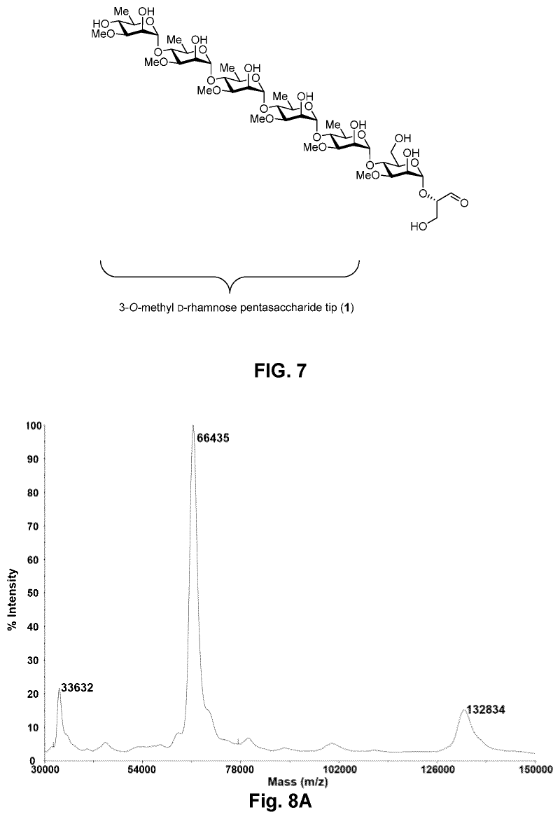

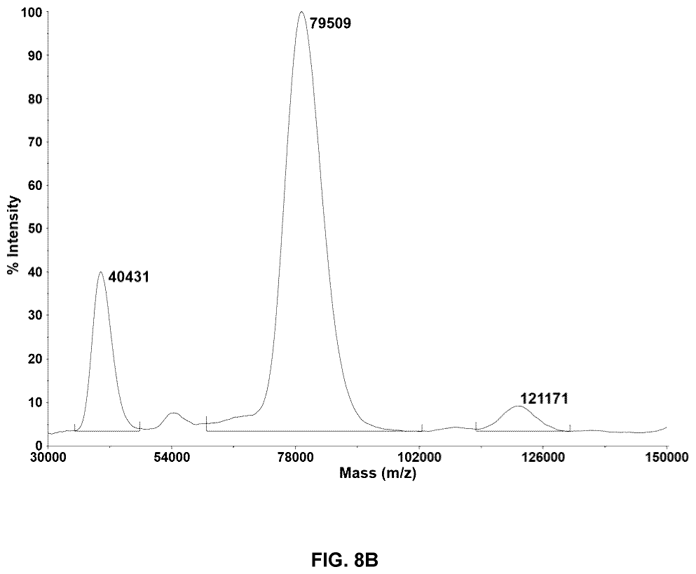

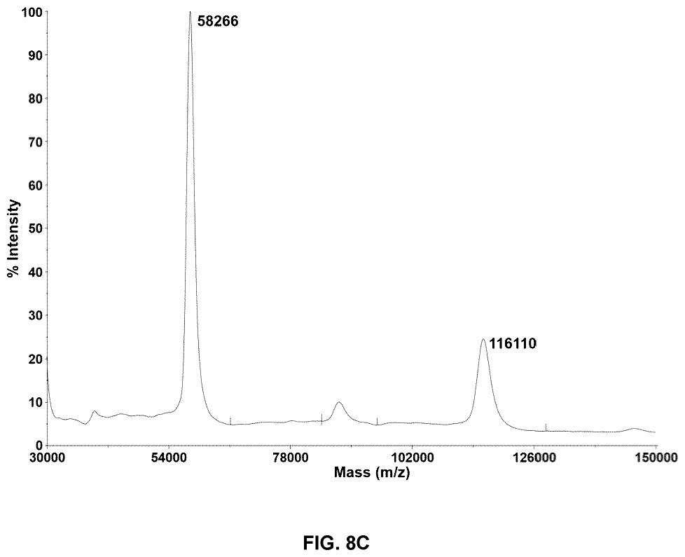

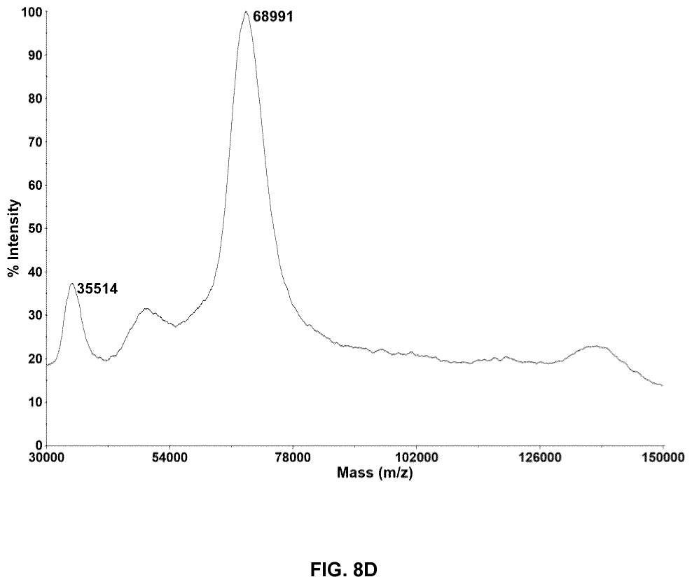

Conjugation: The reducing end erythritol in OS1 was effectively targeted with periodate oxidation in order to create a reactive aldehyde group for conjugation. The oxidised oligosaccharide OS2 (FIG. 7) was conjugated to carrier proteins BSA and CRM₁₉₇ via direct reductive amination and MALDI-MS analysis illustrated that the resulting glycoconjugates contained ˜13 and ˜10 kDa of conjugated carbohydrate (10-13 sugar chains/protein) attached respectively (FIGS. 8A to 8D).

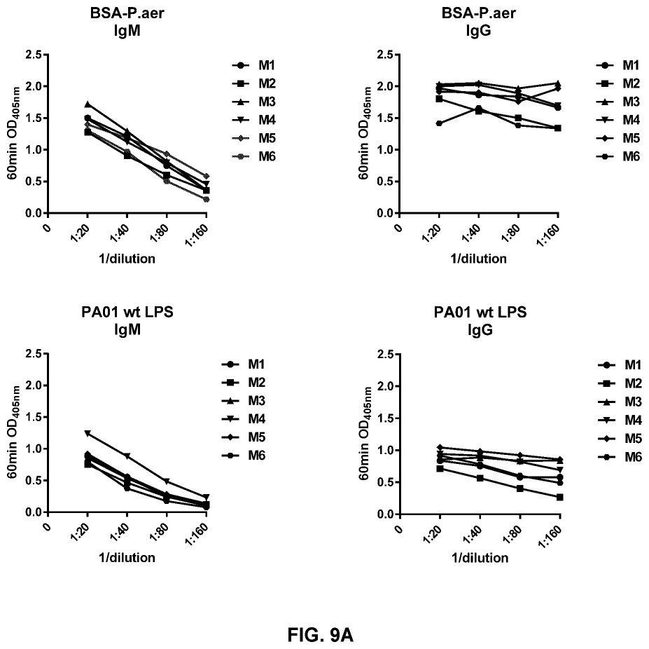

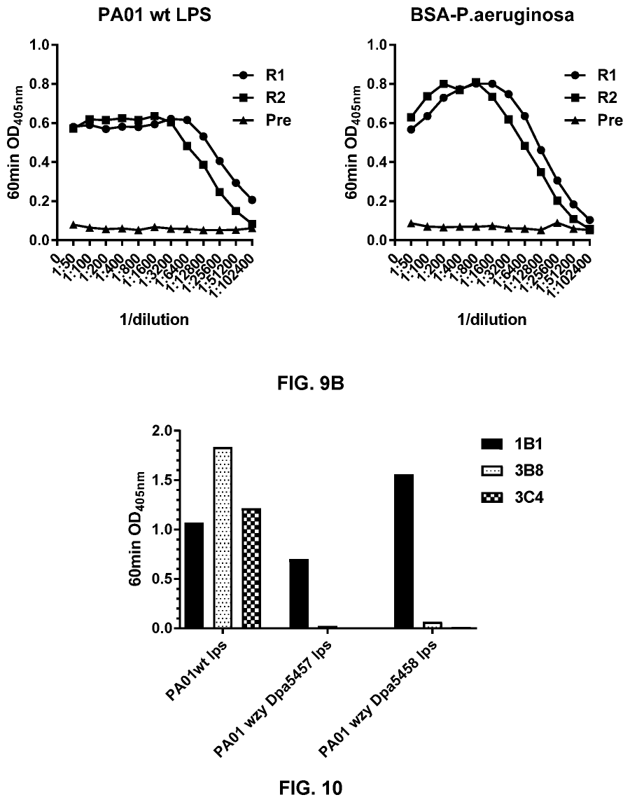

Immunisation: Six mice and two rabbits received a prime and two boost immunisation strategy of the CRM glycoconjugate as described in the Experimental. ELISA with the final bleed sera highlighted that both animals has seroconverted to the conjugated oligosaccharide as evidenced by titrations against the BSA conjugate and LPS (FIGS. 9A and 9B). Mice gave stronger titers to the BSA conjugate than the LPS, whereas rabbits gave broadly similar titration curves to both antigens.

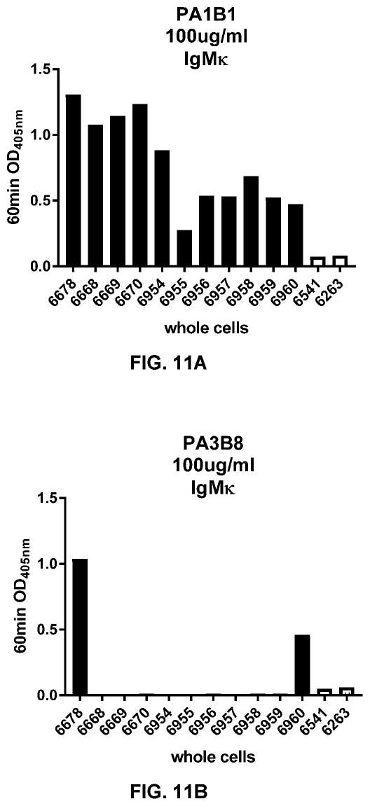

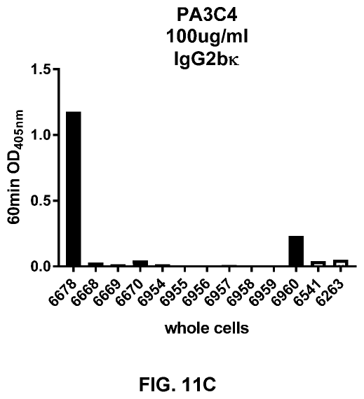

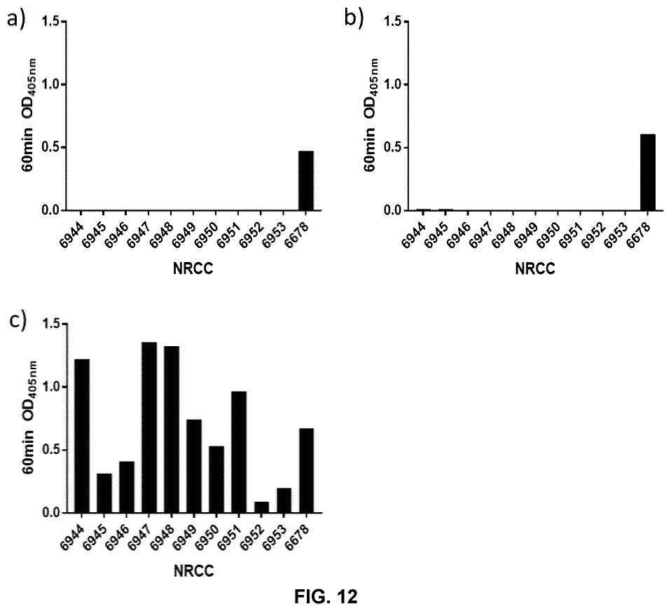

Mab development: A mouse with the best titers to PAO1 LPS (NRCC 6667) following glycoconjugate immunisation was selected for mAb development and its spleen was fused to the myeloid cell line as described in the experimental section. Three mAbs, 3C4 (IgG2b), 3B8 (IgM) and 1B1 (IgM) were obtained. The heavy and light chain variable regions of these mAbs were sequenced and their amino acid sequences are shown in Table 4. Each mAb could recognise the homologous PAO1 (wzy::Gm) LPS and in the case of mAb 1B1, P. aeruginosa purified LPS from the A-band locus mutants PAO1 (wzy::Gm)(Δpa5457) and PAO1 (wzy::Gm)(Δpa5458) (FIG. 10). Similarly, each mAb could recognise killed whole cells of the wt serotype 5 strains PAO1 BAA-47 and 5937, and once again, corroborating the LPS ELISA data, mAb 1B1 was the only mAb that was cross reactive to killed whole cells of the A-band locus mutants PAO1 (wzy::Gm)(Δpa5457), PAO1 (wzy::Gm)(Δpa5458) and PAO1 (wzy::Gm)(Δpa5459) and a sub-set including the ATCC type strains corresponding to the most commonly encountered clinical serotypes (FIGS. 11A to 11C). This behaviour was replicated when a set of clinical isolates was examined (FIG. 12), with mAb 1B1 exhibiting broad cross reactivity.

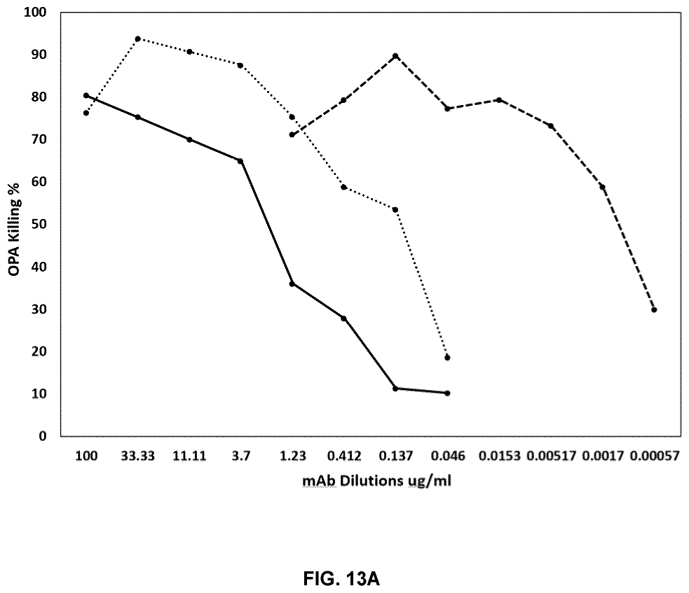

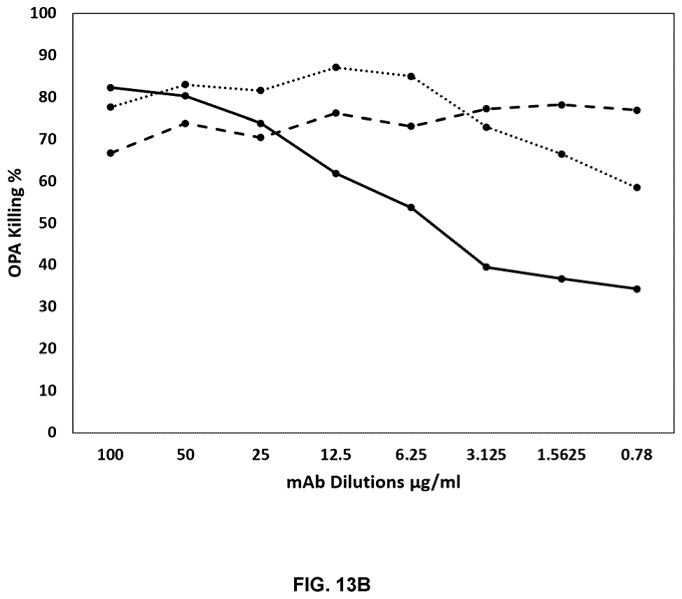

SBA & OPA: Initial efforts were focussed on illustrating the ability of the polyclonal rabbit sera to facilitate serum bactericidal killing of the PAO1 wt P. aeruginosa strain. However, limited evidence of killing was observed with the polyclonal rabbit sera. Subsequent efforts were focussed on the opsonophagocytic assay (OPA), as reports in the literature [8-10] utilise the OPA to illustrate potential functionality of antisera against P. aeruginosa. Similar to the SBA we obtained tentative evidence that the rabbit sera could facilitate opsonophagocytic activity against the PAO1 O-antigen deficient strain. However, when the mAbs were examined, high opsonophagocytic titers were observed against the PAO1 serotype 5 strains PAO1 BAA-47 and 5937 (FIGS. 13A and 13B).

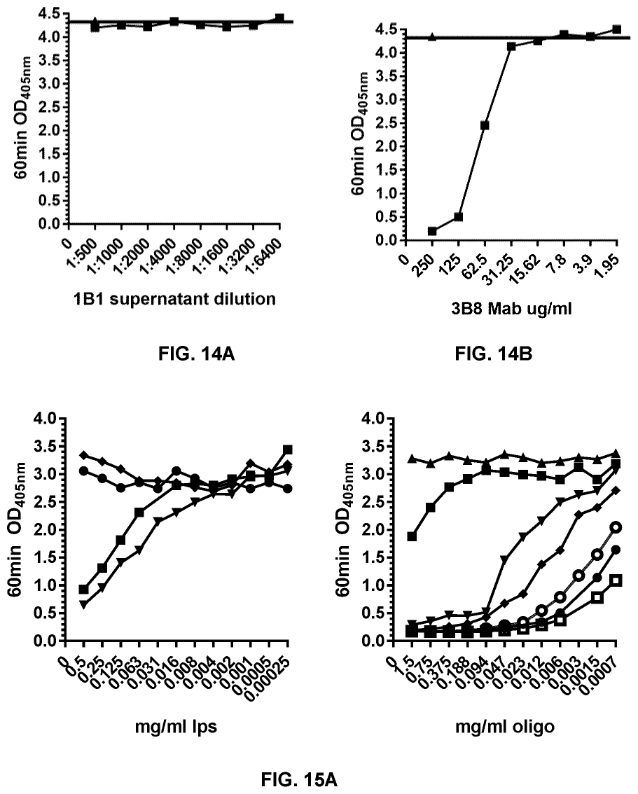

mAb epitope mapping: Preliminary experiments were conducted to examine if the mAbs recognised the same, different or overlapping epitopes on the 3-O-Me rhamnan. Competition ELISA studies suggested that mAb 1B1 recognised a unique epitope compared to 3C4, whereas 3C4 and 3B8 recognised similar overlapping epitopes (FIGS. 14A and 14B), consistent with the ability of the 1B1 mAb to recognise a broader subset of strains compared to mAbs 3B8 and 3C4.

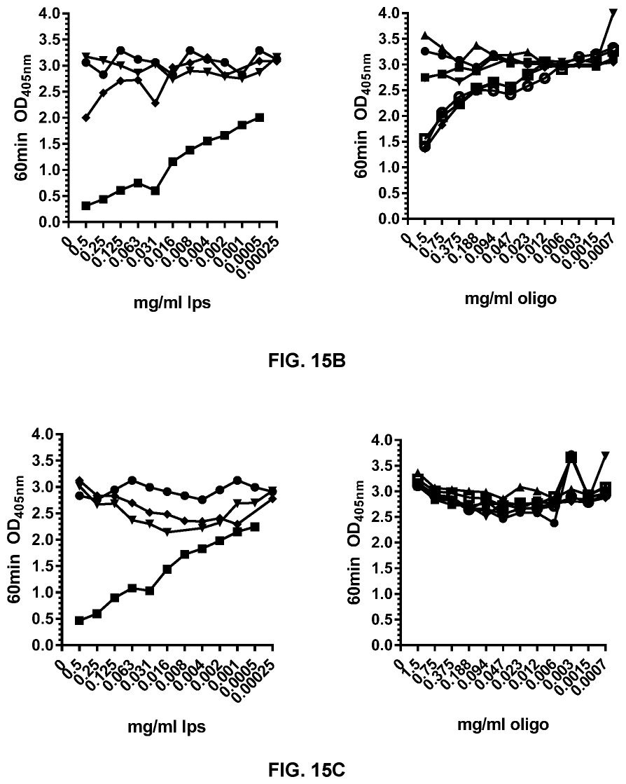

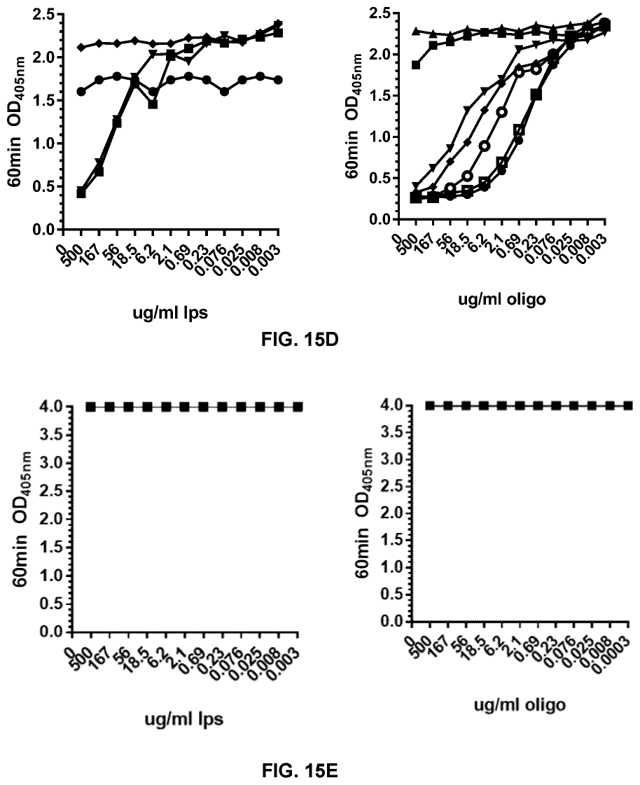

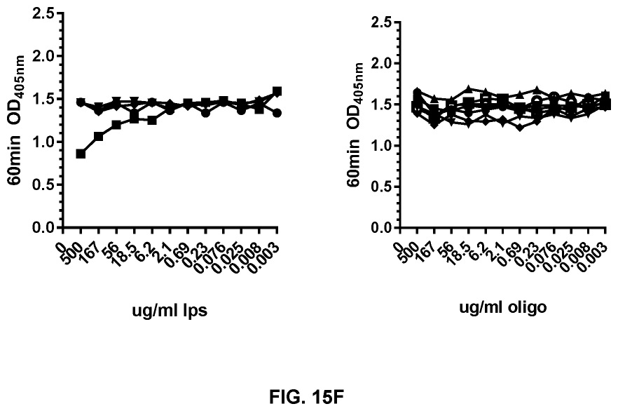

Inhibition ELISA with synthetic oligosaccharides: In order to further characterise the immunogenic epitope recognised by the mAbs, synthetic oligosaccharides representing mono-(D- and L-isomers), di- (with and without a linker), tri-, tetra- and pentasaccharides of the 3-O-methyl D-rhamnan terminal unit were prepared as described herein and examined in an inhibition ELISA experiment. Initially the experiment was validated using LPS molecules known to be recognised or not recognised by the three mAbs. Subsequently the synthetic oligosaccharides were used as inhibitors, revealing that the tri-, tetra- and pentasaccharide were effectively equivalent in inhibiting the binding of mAb 1B1 to its target LPS and killed whole cells (FIGS. 15A to 15F). There was some evidence of inhibition with the di- and monosaccharides (D-isomer only), but not to the extent of that observed with the trisaccharide and larger oligosaccharides. The L-monosaccharide was completely ineffective in inhibition confirming that the D-isomer is the correct configuration of the rhamnose sugar in the 3-O-methyl rhamnan. None of the synthetic oligosaccharides were effective at inhibiting the binding of mAbs 3C4 or 3B8 to their LPS or killed whole cell targets. These results are consistent with the earlier data suggesting that mAb 1B1 can recognise the terminal 3-O-methyl rhamnan and that it is possible that a trisaccharide is sufficient for effective mimicry of the natural structure.

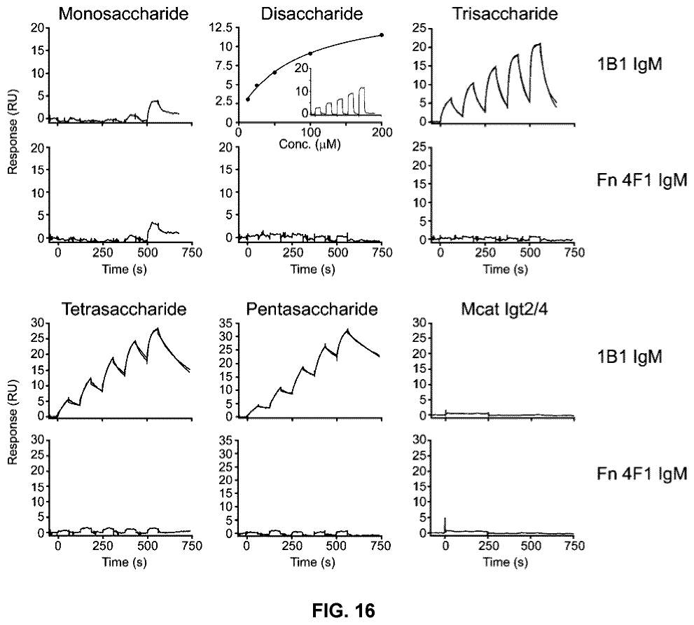

SPR analyses: SPR was used to further delineate the minimum epitope recognized by 1B1 and determine the affinities for synthetic oligosaccharides (FIG. 16 and Table 5). Synthetic carbohydrates ranging from a monosaccharide to pentasaccharide were flowed over high density surfaces of 1B1 IgM and an irrelevant IgM, Fn 4F1. No observable binding was detected for the monosaccharide at up to 1 mM. The disaccharide showed specific binding to the 1B1 surface, with rapid association and dissociation rates, and a steady-state affinity of K_{D}=84 μM was determined. The tri-, tetra- and pentasaccharides all bound 1B1 specifically, with considerably higher affinities than the disaccharide (K_{D}s=1.42, 0.66 and 0.55 μM, respectively). All three synthetic oligosaccharides had measureable kinetics, highlighted by slow dissociation rates not commonly observed in carbohydrate-antibody interactions. Observed Rₘₐₓ values were approximately 25% of theoretical Rₘₐₓ indicating acceptable 1B1 IgM surface activity. No observable binding was detected on either IgM surface when an irrelevant oligosaccharide Mcat lgt2/4 (886 Da) from M. catarrhalis was injected at 10 μM. Taken together these data suggest the trisaccharide is likely the minimal antigen recognition domain mimicking the natural structure, although higher 1B1 binding affinities were observed for the tetra- and pentasaccharides.

TABLE 5
SPR-derived 1B1 binding affinities for synthetic oligosaccharides.

| | Theo | Obs | | | |
|---|---|---|---|---|---|
| Oligosacchari | Rₘₐₓ | Rₘₐₓ | kₐ (s⁻¹) | k_{d} (M⁻¹ s⁻¹) | K_{D} (μM) |
| Mono- | 31 | n.b. | n.b. | n.b. | n.b. |
| | | n.b | n.b. | n.b. | n.b. |
| Di- | 59 | 14 | — | — | 85.8^{a} |
| | | 15 | — | — | 82.5^{a} |
| Tri- | 87 | 21 | 1.28 × 10⁴ | 1.76 × 10⁻² | 1.37 |
| | | 21 | 1.23 × 10⁴ | 1.79 × 10⁻² | 1.46 |
| Tetra- | 115 | 28 | 5.69 × 10³ | 3.72 × 10⁻³ | 0.65 |
| | | 29 | 5.26 × 10³ | 3.48 × 10⁻³ | 0.66 |
| Penta- | 142 | 34 | 3.43 × 10³ | 1.86 × 10⁻³ | 0.54 |
| | | 34 | 5.43 × 10³ | 1.90 × 10⁻³ | 0.55 |

^{a}Steady state affinity.
Theo Rₘₐₓ: theoretical maximum binding response; Obs Rₘₐₓ: observed maximum binding response; n.b.: no binding observed.

### Example 3A—Synthesis of 3-O-methyl Rhamnose Oligosaccharides

This example describes synthesis of target pentasaccharide (see Schemes 1-3 in FIGS. 22-24). The synthesis of 3-O-D-rhamnose and 3-O-L-rhamnose and derivatization to their respective acetylated has-2-octyl glycosides demonstrated the purity of each enantiomer produced via GC-MS. Large scale synthesis of intermediate thioglycoside donor 16 and acceptor 17 led to disaccharide 18 di. After benzyl deprotection, the new acceptor 19 di was obtained and further iterative glycosylation and hydrogenation reactions led ultimately to 18 penta. After global deprotection, the synthesis of the target pentasaccharide resulted in 1.0% overall yield from D-mannose. Additionally, an aminoethyl linker was added to the disaccharide to develop methods for further conjugate vaccine development. The methods described in this example illustrate that pure pentasaccharide can be obtained in sufficient amounts via syntheses to facilitate P. aeruginosa glycoconjugate vaccine development using this glycan.

All chemicals were purchased from Aldrich, Fisher Scientific, Alpha Aeser, or Combi Blocks. They were used without further purification. NMR spectra were measured on a Varian (¹H 500 MHz, ¹³C 125 MHz, ³¹P 200 MHz) spectrometer reported with the solvent residual signal (CDCl₃, 7.26 ppm for ¹H and 77.1 ppm for ¹³C, CD₃OD, 3.31 ppm for ¹H and 49.0 ppm for ¹³C D₂O, 4.79 ppm for ¹H and externally with dioxane (67.2 ppm) for ¹³C and in a separate experiment prior measuring the spectra with 85% H₃PO₄ for ³¹P). Compound assignments were confirmed using standard two dimensional NMR experiments such as HMBC, COSY, HSQC and ¹³C NMR. Compounds were purified using a CombiFlash® RF system and RediSep® RF silica columns. MS data were recorded on a SQ2 from waters and HRMS data were recorded on an Ultima from Waters with the LC/MS Calibrant Mix from Agilent as internal standard.

### 1,4-O-Dibenzyl-2,3-O-isopropylidene-α-D-rhamnopyranoside (4)

1-O-Benzyl-2,3-O-isopropylidene-α-D-rhamnopyranoside (9.70 g, 32.9 mmol) was dissolved in anhydrous DMF (200 mL) and the solution was cooled to 0° C. Sodium hydride 60% in mineral oil (1.97 g, 49.3 mmol) was added in portions and the solution was stirred under nitrogen gas for 30 minutes at 0° C. Next, benzyl bromide (4.70 mL, 39.5 mmol) was added dropwise and the reaction was stirred for 1 h before reaching completion by TLC. Once complete, the reaction was quenched with Et₃N (8 mL), and poured into a cold solution of saturated NH₄Cl (500 mL). As the product crashed out of solution, it was filtered off, washed with water, and dried under high vacuum overnight to generate the product, 4 (12.6 g, 32.9 mmol, 100%) as a beige solid. Rf=0.75, (EtOAc/hexane, 2/8) [α]_{D}²⁵ 23.45 (c 0.22, CHCl₃) ¹H NMR (500 MHz, CDCl₃): δ 7.38-7.26 (m, 10H, 2×Bn), 5.05 (s, 1H, H1α), 4.89 (d, 1H, J_{A,B}=11.6 Hz, CHA, OBn), 4.69 (d, 1H, J_{A,B}=11.8 Hz, CHA, OBn), 4.64 (d, 1H, J_{B,A}=11.6 Hz, CHB, OBn), 4.51 (d, 1H, J_{B,A}=11.8 Hz, CHB, OBn), 4.29 (dd, 1H, J_{3,4}=6.2 Hz, J_{3,2}=6.2 Hz, H3), 4.19 (d, 1H, J_{2,3}=5.8 Hz, H2), 3.80-3.73 (m, 1H, H5), 3.24 (dd, 1H, J_{4,3}=7.2 Hz, J₄,5=9.9 Hz, H4), 1.51, 1.36 (2×s, 3H, CH₃), 1.29 (d, 3H, J_{6,5}=6.3 Hz, H6) ¹³C NMR (125 MHz, CDCl₃): δ 138.4, 137.2, 128.6 (×2), 128.4 (×2), 128.3 (×2), 128.1 (×2), 128.0, 127.7 (Bn), 109.3 (C(CH₃)₂), 96.3 (C1), 81.3 (C4), 78.7 (C3), 76.2 (C2), 73.1, 69.1 (2×CH₂ (Bn), 64.8 (C5), 28.1, 26.4 (2×CH₃), 17.9 (C6) J_{C1, H1}=174 Hz, HRMS: m/z Calcd for C₂₃H₂₈NaO₅ [M+Na]⁺, 407.1834; found, 407.1828.

### 1,4-O-Dibenzyl-α-D-rhamnopyranoside (5)

Compound 4 (0.46 g, 1.21 mmol) was dissolved in a solution of 80% (v/v) AcOH (8 mL) and the solution was heated to 60° C. for 4 h. The solution was then evaporated under reduced pressure, and the crude product was crystalized with CH₂Cl₂/hexane to generate 5 (395 mg, 1.15 mmol, 95%) as white crystals. Rf=0.6 (EtOAc/hexane, 7/3) [α]_{D}²⁵ 1.49 (c 0.09, CHCl₃) ¹H NMR (500 MHz, CDCl₃): δ 7.36-7.26 (m, 1 OH, 2×Bn), 4.86 (s, 1H, H1), 4.78-4.73 (m, 2H, CHA, CHB, OBn), 4.69 (d, 1H, J_{A,B}=11.9 Hz, CHA, OBn), 4.51 (d, 1H, J_{B,A}=11.9 Hz, CHB, OBn), 3.99-3.94 (m, 2H, H2, H3), 3.83-3.76 (m, 1H, H5), 3.37 (dd, 1H, J_{4,3}=J_{4,5}=9.1 Hz, H4), 2.26 (d, 1H, J_{OH,2}=3.6 Hz, OH), 2.24 (d, 1H, J_{OH,3}=4.8 Hz, OH), 1.36 (d, 3H, J_{6,5}=6.3 Hz, H6). ¹³C NMR (125 MHz, CDCl₃): δ 138.3, 137.3, 128.7 (×2), 128.6 (×2), 128.1, 128.1 (×2), 128.0 (×2), 128.0, (Bn), 98.6 (C1), 81.8 (C4), 75.2 (CH₂, Bn), 71.6 (C3), 71.3 (C2), 69.2 (CH₂, Bn), 67.5 (C5), 18.1 (C6) J_{C1, H1}=167 Hz LRMS m/z Calcd for C₂₀H₂₄NaO₅ [M+Na]⁺, 367.4; found, 367.3.

### 1,4-di-O-Benzyl-3-O-methyl-α-D-rhamnopyranoside (6)

Compound 5 (25.1 g, 72.9 mmol) was coevaporated with toluene three times and dried on high vacuum overnight. 5 was then dissolved in anhydrous toluene (500 mL), purged with nitrogen gas and dibutyltin (IV) oxide (21.8 g, 87.5 mmol) was added. The reaction mixture was stirred at reflux for 16 h before being cooled to RT and evaporated under vacuum. The crude mixture was further dried on high vacuum for 5 h before being re-dissolved in 500 mL of anhydrous DMF. The mixture was purged with nitrogen gas, then cesium fluoride (16.5 g, 109 mmol) and iodomethane (51.7 mL, 830.4 mmol) were added. The reaction was stirred under nitrogen gas at 40° C. for 16 h before reaching completion by TLC. The reaction mixture was cooled to RT and diluted with EtOAc (300 mL), washed with water (5×60 mL), saturated NaHCO₃ (1×60 mL), and brine (1×60 mL). The combined organic layers were then dried with Na₂SO₄, evaporated and purified by flash chromatography (eluent: EtOAc/hexane) to yield 6 as a pale oil (23.7 g, 66.3 mmol, 91%—2 steps). Rf=0.35 (EtOAc/hexane, 7/3) [α]_{D}²⁵ 58.3 (c 0.43, CHCl₃) ¹H NMR (500 MHz, CDCl₃): δ 7.37-7.26 (m, 10H, 2×Bn), 4.91 (d, 1H, J_{1,2}=1.2 Hz, H1), 4.86 (d, 1H, J_{A,B}=11.0 Hz, CHA, OBn), 4.71 (d, 1H, J_{A,B}=11.8 Hz, CHA, OBn), 4.64 (d, 1H, J_{B,A}=11.6 Hz, CHB, OBn), 4.47 (d, 1H, J_{B,A}=11.0 Hz, CHB, OBn), 4.08 (dd, 1H, J_{2,1}=1.7 Hz, J_{2,3}=3.3 Hz, H2), 3.83-3.73 (m, 1H, H5), 3.61 (dd, 1H, J_{3,2}=3.4 Hz, J_{3,4}=9.1 Hz, H3), 3.49 (s, 3H, OMe), 3.39 (dd, 1H, J_{4,3}=9.4 Hz, J_{4,5}=9.4 Hz, H4), 2.43 (d, 1H, J_{OH,3}=2.0 Hz, OH), 1.31 (d, 3H, J_{6,5}=6.3 Hz, H6). ¹³C NMR (125 MHz, CDCl₃): δ 138.6, 137.3, 128.5 (×2), 128.5 (×2), 128.1 (×2), 128.1 (×2), 128.0, 127.8 (Bn), 98.3 (C1), 81.8 (C3), 80.0 (C4), 75.4, 69.2 (2×CH₂ (Bn)), 68.0 (C2), 67.4 (C5), 57.5 (OCH₃), 18.0 (C6) J_{C1, H1}=174 Hz HRMS: m/z Calcd for C₂₁H₂₆NaO₅ [M+Na]⁺, 381.1672; found, 381.1668.

### 3-O-Methyl-D-rhamnose (D-monosaccharide)

1-O-Benzyl-3-O-methyl-α-D-rhamnopyranoside (43.7 mg, 162.9 μmol) was dissolved in a 16:10 (V:V) solution of 4 M HCl and MeCN (10 mL) and the solution was heated 80° C. for 16 h. The solution was then evaporated and purified by flash chromatography (eluent: MeOH/CH₂Cl₂, 1/5) to afford monosaccharide as a white solid (18.9 mg, 106.1 μmol, 65%). Rf=0.30, (EtOAc), ¹H NMR (600 MHz, CD₃OD): δ 5.18 (d, 0.7H, J_{1α,2α}=1.6 Hz, H1α), 4.74 (d, 0.3H, J_{1β,2β}=1.0 Hz, H1β), 4.07 (dd, 0.7H, J_{2β,1β}=0.7 Hz, J_{2β,3β}=2.9 Hz, H2β), 4.03 (dd, 0.7H, J_{2α,1α}=0.9 Hz, J_{2α,3α}=1.9 Hz, H2α), 3.89-3.83 (m, 0.7H, H5α), 3.50-3.48 (2×s, 3H, OCH₃ α/β), 3.46 (dd, 0.7H, J_{4α,3α}=3.0 Hz, J_{4α,5α}=9.4 Hz, H4α), 3.43-3.39 (m, 1H, H3α and H4β), 3.42-3.35 (m, 0.3H, H5β), 3.15 (dd, 1H, J_{3β,2β}=3.0 Hz, J_{3β,4β}=9.2 Hz, H3β), 1.34-1.25 (m, 3H, H6α/H6β). ¹³C NMR (151 MHz, CD₃OD): δ 95.7 (C1α), 95.4 (C1β), 84.5 (C3β), 81.8 (C3α), 73.0 (C4α), 72.4 (C4β), 71.1 (C5β), 69.2 (C5α), 69.0 (C2β), 68.8 (C2α), 57.3 (OCH₃ α), 57.2 (OCH₃ β), 18.1 (C6α), 18.1 (C6β). J_{C1, H1α}=172 Hz, J_{C1, H1β}=161 Hz, HRMS: m/z Calcd for C₇H₁₄NaO₅ [M+Na]⁺, 201.0739; found, 201.0734. NMR spectrum is shown in FIG. 5B.

### 3-O-Methyl-L-rhamnopyranose (L-monosaccharide)

1,2-di-O-Acetyl-3-O-methyl-L-rhamnopyranoside S9 (100 mg, 381 μmol) was dissolved in a 10 mL solution of MeOH/H₂O/Et₃N (7/2/1) and the reaction was stirred at RT for 16 h. The solution was then evaporated under reduced pressure and filtered through Dowex® Na⁺, then filtered through a Sephadex® G-12 column. The fractions containing the product were collected and evaporated to yield 13 as an α/β mixture (8/5) as a clear oil (37.5 mg, 210 μmol, 55%), ¹H NMR (600 MHz, CD₃OD): δ 5.06 (d, 0.8H, J_{1α,2α}=1.7 Hz, H1α), 4.87 (d, 0.2H, J_{1β,2β}=1.7 Hz, H1β), 4.05 (d, 0.2H, J_{2β,1β}=0.9 Hz, J_{2β,3β}=3.1 Hz, H2β), 4.01 (dd, 0.8H, J_{2α,1α}=1.8 Hz, J_{2α,3α}=2.9 Hz, H2α), 3.83 (m, 0.8H, H5α), 3.48 (s, 0.6H, OCH₃β), 3.48 (s, 2.4H, OCH₃α), 3.44 (dd, 0.8H, J_{4α,3α}=J_{4α,5α}=9.6 Hz, H4α), 3.41-3.36 (m, 1H, H3α, H4β), 3.29 (m, 0.2H, H3β), 1.30 (d, 0.6H, J_{6α,5β}=6.1 Hz), 1.25 (d, 2.4H, J_{6α,5α}=6.1 Hz, H6α). ¹³C NMR (151 MHz, CD₃OD): δ 95.7 (C1α), 95.3 (C1β), 84.5 (C3β), 81.8 (C3α), 73.0 (C4α), 72.4 (C4β), 73.3 (C5β), 69.2 (C5α), 69.0 (C2β), 68.8 (C2α), 57.3 (OCH₃ α), 57.2 (OCH₃ β), 18.1 (C6α), 18.1 (C6β). J_{C1, H1α}=172 Hz, J_{C1, H1β}=162 Hz. HRMS: m/z Calcd for C₇H₁₄NaO₅ [M+Na]⁺, 201.0733; found, 201.0735. NMR spectrum is shown FIG. 5A.

### 4-O-Benzyl-3-O-methyl-D-rhamnopyranose (14)

Compound 6 (1.54 g, 4.30 mmol) was dissolved in a 16:10 solution of 4 M HCl and MeCN (219 mL) and the solution was heated at 80° C. for 3 h. TLC analysis showed the reaction had reached completion after 3 h. The solution was then evaporated and purified by flash chromatography (eluent: MeOH/CH₂Cl₂, 1/5) to afford compound 14 as an α/β mixture (6/5) as a clear oil (0.75 g, 2.80 mmol, 65%). Rf=0.25 (EtOAc/hexane, 1/1) ¹H NMR (500 MHz, CDCl₃): δ 7.36-7.27 (m, 5H, Bn), 5.25 (s, 1H, H1α), 4.85 (d, 1H, J_{A,B}=11.1 Hz, CHA, OBn), 4.72 (s, 1H, H1β), 4.63 (d, 1H, J_{B,A}=10.9 Hz, CHB, OBn), 4.10-4.08 (m, 2H, H2α, H2β), 3.99-3.92 (m, 1H, H5α), 3.62 (dd, 1H, J_{3α,4α}=9.1 Hz, 3.3 Hz, H3α), 3.52, 3.50 (2×s, 3H, OCH₃ α/β), 3.38 (dd, 1H, J_{4α,3α}=9.4 Hz, J_{4α,5α}=9.4 Hz, H4α), 3.37-3.31 (m, 3H, H3β, H4β, H5β), 2.56 (br. s, 1H, OH), 2.46 (br. s, 1H, OH), 1.33 (d, 3H, J_{6β,5β}=5.7 Hz, H6β), 1.29 (d, 3H, J_{6α,5α}=5.7 Hz, H6α). ¹³C NMR (125 MHz, CDCl₃): δ 128.5, 128.4, 128.3, 128.0, 127.9, 127.8, 127.7 (Bn), 93.9 (C1α), 93.8 (C1β), 83.8 (C3β), 81.3 (C3α), 79.8 (C4α), 79.3 (C4α), 75.2 (CH₂, Bn), 71.1 (C5β), 68.3 (C2α), 68.0 (C2β), 67.3 (C5α), 57.5 (OCH₃α), 57.4 (OCH₃β), 18.0 (CH₃, C6α), 17.9 (CH₃, C6β). HRMS m/z Calcd for C₁₄H₂₀NaO₅ [M+Na]⁺, 291.1203; found, 292.1205.

### p-Tolyl 2-acetyl-4-O-benzyl-3-O-methyl-1-thio-D-rhamnopyranoside (16)

Compound 15 (925 mg, 2.63 mmol) was dissolved in anhydrous CH₂Cl₂ (20 mL) and the solution was cooled to 0° C. under nitrogen gas. Next, thiocresol (456 mg, 3.67 mmol) was added followed by boron trifluoride diethyl etherate (452.9 μL, 3.67 mmol). The reaction was stirred at at 0° C. for 3 h before reaching completion. The solution was then diluted with CH₂Cl₂ (20 mL), and washed with water (2×20 mL) and a saturated NaHCO₃ solution (1×20 mL). The combined organic layers were then dried with Na₂SO₄, evaporated, purified by flash chromatography (eluent: EtOAc/hexane) to yield compound 16 as an α/β mixture (5/2) (880 mg, 2.11 mmol, 80%) as a brown oil. Rf=0.6 (EtOAc/hexane, 3/7) [α]_{D}²⁵ 23.1 (c 2.1, CHCl₃) ¹H NMR (500 MHz, CDCl₃): δ 7.43-7.28 (m, 7H, Bn/Stol), 7.20-7.10 (m, 2H, Stol), 5.71 (d, 0.5H, J_{2β,3β}=2.5 Hz, H2β), 5.56 (dd, 0.5H, J_{2α,3α}=1.6 Hz, 3.2 Hz, H2α), 5.35 (d, 0.5H, J_{1α,2α}=1.3 Hz, H1α), 4.92 (d, 0.5H, J_{Aα,Bα}=11.0 Hz, CHAα, OBn), 4.87 (d, 0.5H, J_{Aβ,Bβ}=11.0 Hz, CHAβ, OBn), 4.75 (s, 0.5H, H1β), 4.64 (d×2, 1H, J_{Bα,Aα and Bβ,Aβ}=10.7 Hz, CHBα and CHBβ, OBn), 4.27-4.20 (m, 0.5H, H5α), 3.65 (dd, 1H, J_{3α,4α}=9.3 Hz, J_{3α,2α}=3.3 Hz, H3α), 3.47 (s, 1.5H, OCH₃α), 3.45 (s, 1.5H, OCH₃β), 3.45-3.42 (m, 0.5H, H4α), 3.40-3.34 (m, 1.5H, H3β, H4β, H5β), 2.34 (s, 1.5H, CH₃, STol β), 2.33 (s, 1.5H, CH₃, STol α), 2.23 (s, 1.5H, Ac β), 2.15 (s, 1.5H, Ac α), 1.40 (d, 1.5H, J_{6β,5β}=5.5 Hz, H6β), 1.35 (d, 1.5H, J_{6α,5α}=6.2 Hz, H6α). ¹³C NMR (125 MHz, CDCl₃): δ 170.5 (COβ), 170.3 (COα), 138.6, 138.5, 138.1, 138.0, 132.3 (×2), 132.3 (×2), 130.2, 130.2, 129.9 (×2), 129.9, 129.1, 128.5 (×2), 128.5, 128.3, 128.1, 128.0 (×2), 127.8, 127.8, 125.4 (Ar), 86.5 (C1α), 85.8 (C1β), 84.0 (C3β), 80.5 (C3α), 80.4 (C4α), 79.3 (C4β), 76.1 (C5β), 75.4 (CH₂, Bn α, Bn β), 70.4 (C2α), 70.0 (C2β), 68.9 (C5α), 57.8 (OCH₃ β), 57.7 (OCH₃ α), 21.2 (CH₃, Stol β), 21.2 (CH₃, Stol α), 21.1 (Ac α), 20.9 (Ac β), 18.3 (C6β), 17.9 (C6α). J_{C1, H1α}=169 Hz, J_{C1, H1β}=154 Hz, ESI-MS: m/z Calcd for C₂₃H₂₈NaO₅S [M+Na]⁺, 439.1550; found, 439.1545.

### 1,2-Diacetyl-3-O-methyl-α-D-rhamnopyranoside (17)

Compound 15 (450 mg, 1.28 mmol) was dissolved in 10.0 mL of MeOH and the solution was charged with palladium hydroxide on carbon (40 mg, 0.28 mmol). The solution was bubbled with a balloon of hydrogen gas for 5 minutes and stirred under an atmosphere of hydrogen gas for 3 h before reaching completion. The solution was then filtered through celite and evaporated to yield pure 17 (323 mg, 1.23 mmol, 96%) as a clear oil. Rf=0.25 (EtOAc/hexane, 1/1) [α]_{D}²⁵−21.2 (c 3.4, CHCl₃) ¹H NMR (500 MHz, CDCl₃): δ=5.96 (s, 1H, H1), 5.24 (d, 1H, J_{2,1}=2.0 Hz, H2), 3.76-3.67 (m, 1H, H5), 3.48 (dd, 1H, J_{4,5}=9.4 Hz, J_{4,3}=9.4 Hz, H4), 3.43 (dd, 1H, J_{3,2}=3.2 Hz, J_{3,4}=9.5 Hz, H3), 3.37 (s, 3H, OCH₃), 2.96 (br. s, 1H, OH), 2.08, 2.07 (2×s, 6H, CH₃), 1.28 (d, 3H, J_{6,5}=6.3, H6) ¹³C NMR (125 MHz, CDCl₃): δ 169.9, 168.5 (2×C═O), 91.2 (C1), 79.3 (C3), 71.1 (C4), 70.4 (C5), 66.4 (C2), 57.4 (OCH₃), 20.9, 20.7 (2×Ac), 17.7 (C6). J_{C1, H1}=170 Hz, LRMS m/z Calcd for C₁₁H₁₈NaO₇ [M+Na]⁺, 285.1; found, 285.1.

### 2-O-Acetyl-4-O-benzyl-3-O-methyl-α-D-rhamnopyranoside-(1→4)-1,2-O-diacetyl-3-O-methyl-α-D-rhamnopyranoside (18 di)

Compounds 16 (615 mg, 1.48 mmol) and 17 (323 mg, 1.23 mmol) were coevaporated together with toluene (5×10 mL) and left to dry on high vacuum overnight. Anhydrous CH₂Cl₂ (20 mL) was added followed by 0.6 g of activated powdered 3 Å molecular sieves. The reaction was then cooled to −78° C. under an atmosphere of nitrogen gas and N-iodosuccinimide (406 mg, 1.80 mmol) was added followed by triflic acid (20 μL, 226 μmol). The reaction was stirred for 3 h before reaching completion. The reaction was then filtered through a Buchner funnel and diluted with 20 mL of CH₂Cl₂, washed with 10% Na₂S₂O₃ (2×20 mL) and a saturated NaHCO₃ solution (1×20 mL). The organic layer was then dried with Na₂SO₄, filtered, and purified by flash chromatography (eluent: EtOAc/hexane) to yield compound 18 di (363 mg, 655 μmol, 53%) as a clear oil. Rf=0.55 (EtOAc/hexane, 1/1) [α]_{D}²⁵ 183 (c 18.7, CHCl₃) ¹H NMR (500 MHz, CDCl₃) δ 7.40-7.16 (m, 5H, Bn), 6.02 (d, 1H, J_{1,2}=1.7 Hz, H1), 5.39 (dd, 1H, J_{2′,3′}=1.9 Hz, 3.0 Hz, H2′), 5.31 (dd, 1H, J_{2,1}=1.7, 1.7 Hz, H2), 5.17 (d, 1H, J_{1′,2′}=1.7 Hz, H1′), 4.92 (d, 1H, J_{A,B}=10.9 Hz, CHA, OBn), 4.65 (d, 1H, J_{B,A}=10.9 Hz, CHB, OBn), 3.88-3.81 (m, 1H, H5), 3.79-3.72 (m, 1H, H5′), 3.64-3.59 (m, 3H, H3, H3′, H4), 3.48, 3.45 (2×s, 6H, OCH₃), 3.40 (dd, 1H, J_{4′,5′}=J_{4′3′}=9.5 Hz, H4′), 2.17, 2.16, 2.15 (3×s, 9H, Ac), 1.35 (d, 3H, J_{6′,5′}=6.2 Hz, H6′), 1.34 (d, 3H, J_{6,5}=6.1 Hz, H6). ¹³C NMR (125 MHz, CDCl₃): δ 170.2, 170.0, 168.6 (3×C═O), 138.5, 128.5 (×2), 128.1 (×2), 127.8 (Bn), 99.5 (C1′), 91.1 (C1), 80.0 (C4, C3′), 79.9 (C4′), 77.9 (C3), 75.5 (CH₂, Bn), 69.3 (C5′), 68.7 (C5), 68.6 (C2′), 66.9 (C2), 57.6, 57.6 (2×OCH₃), 21.2, 21.0, 20.9 (3×Ac), 18.3 (C6), 18.0 (C6′). J_{C1, H1}=173 Hz, J_{C1′, H1′}=179 Hz LRMS m/z Calcd for C₂₇H₃₈NaO₁₂ [M+Na]⁺, 577.2; found, 577.9.

### 2-O-Acetyl-3-O-methyl-α-D-rhamnopyranoside-(1→4)-1,2-O-diacetyl-3-O-methyl-α-D-rhamnopyranoside (19 di)

Same procedure as 17. Compound 18 di (363 mg, 655 μmol), palladium hydroxide on carbon (40 mg, 285 μmol), MeOH (10 mL). Compound 19 di (273 mg, 588 μmol, 92%) was isolated as a clear oil. Rf=0.2 (EtOAc/hexane, 1/1) [α]_{D}²⁵ 44 (c 4.3, CHCl₃) ¹H NMR (500 MHz, CDCl₃) δ 5.93 (d, 1H, J_{1,2}=1.6 Hz, H1), 5.31 (dd, 1H, J_{2,1}=1.9 Hz, J_{2,3}=2.8 Hz, H2′), 5.22 (m, 1H, H2), 5.09 (d, 1H, J_{1′,2}′=1.7 Hz, H1′), 3.76-3.66 (m, 2H, H5, H5′), 3.54-3.51 (m, 2H, H3, H4), 3.44 (dd, 1H, J_{4′,5′}=9.4 Hz, J_{4′,3′}=9.4 Hz, H4′), 3.35, 3.34 (2×s, 6H, OCH₃), 3.34-3.29 (m, 1H, H3′), 2.88 (br. s, 1H, OH), 2.08, 2.06, 2.05 (3×s, 9H, Ac), 1.29-1.25 (m, 6H, H6′, H6). ¹³C NMR (125 MHz, CDCl₃): δ 170.1, 170.0, 168.5 (3×C═O), 99.7 (C1′), 91.0 (C1), 79.9 (C4), 79.4 (C3′), 77.5 (C3), 71.4 (C4′), 69.2 (C5′), 69.0 (C5), 67.4 (C2), 66.8 (C2′), 57.5, 57.2 (OCH₃), 21.0, 20.9, 20.8 (3×Ac), 18.2 (C6), 17.5 (C6′). J_{C1,H1}=173 Hz, J_{C1′,H1′}=168 Hz LRMS m/z Calcd for C₂₀H₃₂NaO₁₂ [M+Na]⁺, 487.2; found, 486.9.

### 2-O-Acetyl-4-O-benzyl-3-O-methyl-α-D-rhamnopyranoside-(1→4)-2-O-acetyl-3-O-methyl-α-D-rhamnopyranoside-(1→4)-1,2-O-diacetyl-3-O-methyl-α-D-rhamnopyranoside (18 tri)

Compounds 16 (243 mg, 584 μmol) and 19 di (226 mg, 487 μmol) were co-evaporated together with toluene (5×10 mL) and left to dry on high vacuum overnight. Anhydrous CH₂Cl₂ (10 mL) was added followed by 0.3 g of activated powdered 3 Å molecular sieves. The reaction was then cooled to −78° C. under an atmosphere of nitrogen gas and N-iodosuccinimide (161 mg, 715 μmol) was added followed by triflic acid (10 μL, 115 μmol). The reaction was stirred for 3 h before reaching completion. The reaction was then filtered through a Buchner funnel and diluted with 20 mL of CH₂Cl₂, washed with 10% Na₂S₂O₃ (2×10 mL) and a saturated sodium bicarbonate solution (1×10 mL). The organic layer was then dried with Na₂SO₄, filtered, and purified by flash chromatography (eluent: EtOAc/Hexane) to yield compound 18 tri (254 mg, 336 μmol, 69%) as a clear oil. Rf=0.55 (EtOAc/hexane, 1/1), [α]_{D}²⁵ 157 (c 15, CHCl₃) ¹H NMR (500 MHz, CDCl₃): δ 7.36-7.25 (m, 5H, Bn), 5.98 (d, 1H, J_{1,2}=1.7 Hz, H1), 5.36-5.32 (m, 2H, H2′, H2″), 5.28 (m, 1H, H2), 5.11 (m, 2H, H1′, H1″), 4.87 (d, 1H, J_{A,B}=10.9 Hz, CHA, OBn), 4.62 (d, 1H, J_{B,A}=10.9 Hz, CHB, OBn), 3.85-3.78 (m, 1H, H5″), 3.78-3.70 (m, 2H, H5′, H5), 3.60-3.54 (m, 3H, H3′, H3″, H4), 3.54-3.47 (m, 2H, H3, H4′), 3.43 (s, 3H, OCH₃), 3.39 (s, 6H, OCH₃×2), 3.36 (dd, 1H, J_{4″,3″}=9.6 Hz, H4″), 2.12 (s, 6H, Ac×2), 2.11 (s, 3H, Ac), 2.08 (s, 3H, Ac), 1.31 (d, 3H, J_{6″,5″}=6.2 Hz, H6″), 1.30 (d, 3H, J_{6,5}=6.3 Hz, H6), 1.29 (d, 3H, J_{6′,5′}=6.3 Hz, H6′). ¹³C NMR (125 MHz, CDCl₃): δ 170.1, 170.0, 169.9, 168.4 (C═O), 138.5, 128.4 (×2), 128.0 (×2), 127.7 (Bn), 99.4 (C1′), 99.3 (C1″), 91.0 (C1), 79.9 (C4″), 79.9 (C3), 79.8 (C3′), 79.8 (C3″), 78.2 (C4′), 78.0 (C4), 75.3 (CH₂, Bn), 69.1 (C5), 68.6 (C2′), 68.4 (C2″), 67.8 (C5′), 67.8 (C5″), 66.7 (C2), 57.5 (OCH₃), 57.2 (OCH₃), 21.1, 21.0, 20.9, 20.8 (Ac), 18.2 (C6″), 18.0 (C6′), 17.8 (C6) J_{C1,H1}=179 Hz, J_{C1′,H1′}=179 Hz, J_{C1″, H1″}=179 Hz HRMS m/z Calcd for C₃₆H₅₂NaO₁₇ [M+Na]⁺, 779.3102; found, 779.3079.

### 2-O-Acetyl-3-O-methyl-α-D-rhamnopyranoside-(1→4)-2-O-acetyl-3-O-methyl-α-D-rhamnopyranoside-(1→4)-1,2-O-diacetyl-3-O-methyl-α-D-rhamnopyranoside (19 tri)

Same procedure as 17. Compound 18 tri (228 mg, 301 μmol), palladium hydroxide on carbon (40 mg, 285 μmol), MeOH (10 mL). Compound 19 tri (195.5 mg, 293 μmol, 97%) was isolated as a clear oil. Rf=0.25 (EtOAc/hexane, 1/1) [α]_{D}²⁵ 118 (c 13.3, CHCl₃) ¹H NMR (500 MHz, CDCl₃): δ 5.98 (d, 1H, J_{1,2}=1.4 Hz, H1), 5.36-5.32 (m, 2H, H2′, H2″), 5.2 (m, 1H, H2), 5.14 (d, 1H, J_{1′,2}′=0.9 Hz, H1′), 5.10 (d, 1H, J_{1″,2″}=1.2 Hz, H1″), 3.82-3.72 (m, 3H, H5, H5′, H5″), 3.58-3.52 (m, 3H, H3, H3′, H4′), 3.52-3.47 (m, 2H, H3″, H4″), 3.40, 3.39, 3.38 (3×s, 3H, OCH₃), 3.41-3.36 (m, 1H, H4), 2.48 (br. s, 1H, OH), 2.13 (s, 3H, Ac), 2.11 (s, 3H, Ac), 2.09 (s, 3H, Ac), 2.08 (s, 3H, Ac), 1.35-1.29 (m, 9H, H6, H6′, H6″). ¹³C NMR (125 MHz, CDCl₃): δ 170.1, 170.1, 170.0, 168.5 (4×C═O), 99.6 (C1′), 99.5 (C1″), 91.1 (C1), 80.0 (C3″), 79.9 (C3), 79.4 (C3′), 78.1 (C4), 78.0 (C4′), 71.6 (C4″), 69.2 (C5), 68.9 (C5′), 67.9 (C5″), 67.8 (C2″), 67.4 (C2′), 66.8 (C2), 57.6, 57.3, 57.2 (3×OCH₃), 21.1 (×2), 21.0, 20.9 (4×Ac), 18.3 (C6″), 18.1 (C6′), 17.6 (C6). J_{C1,H1}=172 Hz, J_{C1′, H1′}=171 Hz, J_{C1″, H1″}=171 Hz LRMS m/z Calcd for C₂₉H₄₆NaO₁₇ [M+Na]⁺, 689.3; found, 688.8.

### 2-O-Acetyl-4-O-benzyl-3-O-methyl-α-D-rhamnopyranoside-(1→4)-2-O-acetyl-3-O-methyl-α-D-rhamnopyranoside-(1→4)-2-O-acetyl-3-O-methyl-α-D-rhamnopyranoside-(1→4)-1,2-O-diacetyl-3-O-methyl-α-D-rhamnopyranoside (18 tetra)

Compounds 16 (146 mg, 351 μmol) and 19 tri (195 mg, 293 μmol) were co-evaporated together with toluene (5×10 mL) and left to dry on high vacuum overnight. Anhydrous CH₂Cl₂ (10 mL) was added followed by 0.2 g of activated powdered 3 Å molecular sieves. The reaction was then cooled to −78° C. under an atmosphere of nitrogen gas and N-iodosuccinimide (96.5 mg, 429 μmol) was added followed by triflic acid (5 μL, 57.5 μmol). The reaction was stirred for 3 h before reaching completion. The reaction was then filtered through a Buchner funnel and diluted with 10 mL of CH₂Cl₂, washed with 10% Na₂S₂O₃ (2×5 mL) and a saturated NaHCO₃ solution (1×5 mL). The organic layer was then dried with Na₂SO₄, filtered, and purified by flash chromatography (eluent: EtOAc/hexane) to yield compound 18 tetra (149.7 mg, 156 μmol, 53%) as a clear oil. Rf=0.55 (EtOAc/hexane, 1/1) [α]_{D}²⁵ 54.7 (c 5.0, CHCl₃) ¹H NMR (500 MHz, CDCl₃): δ 7.37-7.26 (m, 5H, Bn), 5.99 (d, 1H, J_{1,2}=1.7 Hz, H1), 5.37-5.32 (m, 3H, H2′, H2″, H2′″), 5.28 (m, 1H, H2), 5.11 (s, 3H, H1′, H1″, H1′″), 4.86 (d, 1H, J_{A,B}=10.9 Hz, CHA, OBn), 4.62 (d, 1H, J_{B,A}=10.9 Hz, CHB, OBn), 3.85-3.71 (m, 4H, H5, H5′, H5″, H5′″), 3.61-3.53 (m, 4H, H3, H4, H4′, H4″), 3.53-3.49 (m, 3H, H3′, H3″, H3′″), 3.44, 3.40, 3.39, 3.38 (4×s, 12H, OCH₃), 3.37-3.31 (m, 1H, H4′″), 2.13, 2.12 (2×s, 6H, Ac), 2.11, 2.08, 2.07 (3×s, 9H, Ac), 1.34 (d, 3H, J_{6,5}=6.2 Hz, H6), 1.31 (d, 3H, J_{6′,5′}=6.3 Hz, H6′), 1.30 (d, 3H, J_{6″,5″}=6.3 Hz, H6″), 1.28 (d, 3H, J_{6′″,5′″}=6.3 Hz, H6′″). ¹³C NMR (125 MHz, CDCl₃): δ 170.1, 170.0, 170.0, 169.9, 168.5 (C═O), 138.5, 128.4 (×2), 128.0 (×2), 127.8, (Bn), 99.5 (C1′″), 99.4 (C1″), 99.3 (C1′), 91.0 (C1), 80.0 (C3), 80.0 (C3′), 79.8 (C3″, C3′″ and C4′″), 78.3 (C4), 78.1 (C4′ and C4″), 75.4 (CH₂, Bn), 69.1 (C5), 68.6 (C2′″), 68.4 (C5′″), 67.9 (C2″), 67.8 (C2′), 67.7 (C5′), 67.7 (C5″), 66.8 (C2), 57.5, 57.5, 57.3, 57.2 (4×OCH₃), 21.1, 21.0, 21.0, 21.0, 20.9 (5×Ac), 18.3 (C6), 18.1 (C6′), 18.1 (C6″), 18.0 (C6′″). J_{C1,H1}=185 Hz, J_{C1′,H1′}=179 Hz, J_{C1″,H1″}=179 Hz, J_{C1″,H1″}=179 Hz, HRMS m/z Calcd for C₄₅H₆₆NaO₂₂ [M+Na]⁺, 981.3943; found, 981.3910.

### 2-O-Acetyl-3-O-methyl-α-D-rhamnopyranoside-(1→4)-2-O-acetyl-3-O-methyl-α-D-rhamnopyranoside-(1→4)-2-O-acetyl-3-O-methyl-α-D-rhamnopyranoside-(1→4)-1,2-O-diacetyl-3-O-methyl-α-D-rhamnopyranoside (19 tetra)

Same procedure as 17. Compound 18 tetra (110 mg, 114.7 μmol), palladium hydroxide on carbon (20.0 mg, 143 μmol), MeOH (10 mL). Compound 19 tetra (95.6 mg, 110.0 μmol, 96%) was isolated as a white solid. Rf=0.40 (EtOAc/hexane, 1/1) [α]_{D}²⁵ 52.40 (c 4.78, CHCl₃) ¹H NMR (600 MHz, CD₃OD): δ 5.96 (d, 1H, J_{1,2}=1.6 Hz, H1), 5.41 (dd, 1H, J_{2′,1′}=3.0 Hz, J_{2′,3′}=5.6 Hz, H2′), 5.40 (dd, 1H, J_{2″,1″}=3.0 Hz, J_{2″,3″}=5.1 Hz, H2″), 5.37 (s, 1H, H2′″), 5.33 (dd, J_{2,1}=3 Hz, J_{2,3}=5.8 Hz, H2), 5.08 (m, 2H, H1′, H1′″), 5.07 (m, 1H, H1″), 3.88-3.80 (m, 3H, H5, H5′, H5″), 3.80-3.73 (m, 1H, H5′″), 3.69 (dd, 1H, J_{3,2}=3.9 Hz, J_{3,4}=11.2 Hz, H3), 3.61-3.55 (m, 2H, H3′, H3″), 3.55-3.45 (m, 3H, H4, H4′, H4″), 3.42-3.32 (m, 2H, H3′″, H4′″), 3.41 (2×s, 3H, OCH₃), 3.40 (2×s, 3H, OCH₃), 2.15, 2.13, 2.11, 2.11, 2.09 (5×s, 15H, Ac), 1.34-1.26 (m, 12H, H6, H6′, H6″, H6′″). ¹³C NMR (125 MHz, CD₃OD/CDCl₃, 1/1): δ 171.0, 170.9, 170.9, 170.7, 169.3 (C═O), 99.9 (C1′″), 99.8 (C1″), 99.8 (C1′), 91.4 (C1), 80.4 (C3″), 80.3 (C3′), 80.2 (C3), 79.4 (C3′″), 78.9 (C4), 78.8 (C4′), 78.5 (C4″), 71.8 (C4′″), 69.8 (C5′″), 69.5 (C5), 68.6 (C2′″), 68.5 (C2′), 68.4 (C2″), 68.2 (C5′, C5″), 67.3 (C2), 57.7 (×2), 57.5 (×2) (4×OCH₃), 21.0, 21.0, 21.0, 20.9, 20.9 (5×Ac), 18.5 (C6′), 18.3 (C6″), 18.2 (C6′″), 17.6 (C6) J_{C1,H1}=179 Hz, J_{C1′,H1′}=177 Hz, J_{C1″,H1″}=177 Hz, J_{C1″,H1″}=176 Hz HRMS m/z Calcd for C₃₈H₆₀NaO₂₂ [M+Na]⁺, 891.3474; found, 891.3429.

### 2-O-Acetyl-4-O-benzyl-3-O-methyl-α-D-rhamnopyranoside-(1→4)-2-O-acetyl-3-O-methyl-α-D-rhamnopyranoside-(1→4)-2-O-acetyl-3-O-methyl-α-D-rhamnopyranoside-(1→4)-2-O-acetyl-3-O-methyl-α-D-rhamnopyranoside-(1→4)-1,2-O-diacetyl-3-O-methyl-α-D-rhamnopyranoside (18 penta)

Compounds 16 (37.4 mg, 89.9 μmol) and 19 tetra (48.8 mg, 56.2 μmol) were co-evaporated together with toluene (5×10 mL) and left to dry on high vacuum overnight. Anhydrous CH₂Cl₂ (5 mL) was added followed by 50 mg of activated powdered 3 Å molecular sieves. The reaction was then cooled to −78° C. under an atmosphere of nitrogen gas and N-iodosuccinimide (24.7 mg, 109.8 μmol) was added followed by triflic acid (10 μL, 113 μmol). The reaction was stirred for 3 h before reaching completion. The reaction was then filtered through a Buchner funnel and diluted with 10 mL of CH₂Cl₂, washed with 10% Na₂S₂O₃ (2×5 mL) and a saturated NaHCO₃ solution (1×5 mL). The organic layer was then dried with Na₂SO₄, filtered, and purified by flash chromatography (eluent: EtOAc/hexane) to yield compound 18 penta (35.6 mg, 30.7 μmol, 55%) as a clear oil. Rf=0.70 (EtOAc/hexane, 7/3) [α]_{D}²⁵ 22.26 (c 1.75, CHCl₃) ¹H NMR (500 MHz, CDCl₃): δ 7.36-7.26 (m, 5H, Bn), 5.98 (d, 1H, J_{1,2}=1.5 Hz, H1), 5.35-5.31 (m, 4H, H2′, H2″, H2′″, H2″″), 5.27 (s, 1H, H2), 5.10 (s, 4H, H1′, H1″, H1′″, H1″″), 4.86 (d, 1H, J_{A,B}=10.9 Hz, CHA, OBn), 4.61 (d, 1H, J_{B,A}=10.9 Hz, CHB, OBn), 3.84-3.70 (m, 5H, H5, H5′, H5″, H5′″, H5″″), 3.60-3.51 (m, 5H, H3, H4, H4′, H4″, H3″″), 3.51-3.46 (m, 4H, H3′, H3″, H3′″, H4′″), 3.43, 3.39 (2×s, 3H, OCH₃), 3.38 (s, 6H, OCH₃×2), 3.37 (s, 3H, OCH₃), 3.37-3.31 (m, 1H, H4″″), 2.12, 2.12, 2.11, 2.08 (4×s, 12H, Ac), 2.07 (s, 6H, Ac×2), 1.36-1.26 (m, 15H, H6, H6′, H6″, H6′″, H6″″). ¹³C NMR (125 MHz, CDCl₃): δ 170.2, 170.1, 170.1, 170.0, 169.9, 168.5 (C═O), 138.5, 128.4 (×2), 128.0 (×2), 127.8 (Bn), 99.5 (C1′″), 99.4 (C1″), 99.4 (C1′), 99.3 (C1″″), 91.0 (C1), 80.0 (C3″″), 80.0 (C3), 79.9 (C3′), 79.9 (C3″, C3′″, C4″″), 78.3 (C4), 78.3 (C4′), 78.2 (C4″), 78.1 (C4′″), 75.4 (CH₂, Bn), 69.1 (C5), 68.7 (C2″″), 68.4 (C5″″), 67.9 (C2″), 67.9 (C2′″), 67.8 (C2′), 67.8 (C5′), 67.7 (C5″, C5′″), 66.8 (C2), 57.5, 57.5, 57.3, 57.3, 57.2 (5×OCH₃), 21.1, 21.1, 21.0, 21.0, 21.0, 20.9 (6×Ac), 18.3 (C6), 18.1 (C6′), 18.1 (C6″), 18.1 (C6′″), 17.9 (C6″″) J_{C1,H1}=174 Hz, J_{C1′,H1′}=177 Hz, J_{C1″,H1″}=177 Hz, J_{C1′″,H1′″}=177 Hz, J_{C1″″,H1″″}=177 Hz HRMS m/z Calcd for C₅₄H₈₀NaO₂₇ [M+Na]⁺, 1183.4785; found, 1180.4753.

### 2-O-Acetyl-3-O-methyl-α-D-rhamnopyranoside-(1→4)-2-O-acetyl-3-O-methyl-α-D-rhamnopyranoside-(1→4)-2-O-acetyl-3-O-methyl-α-D-rhamnopyranoside-(1→4)-2-O-acetyl-3-O-methyl-α-D-rhamnopyranoside-(1→4)-1,2-O-diacetyl-3-O-methyl-α-D-rhamnopyranoside (19 penta)

Same procedure as 17. Compound 18 penta (35.0 mg, 30.1 μmol), palladium hydroxide on carbon (15.0 mg, 105.0 μmol), MeOH (5 mL). Compound 19 penta (32.2 mg, 30.1 μmol, 100%) was isolated as a white solid. Rf=0.45 (EtOAc/hexane, 7/3) [α]_{D}²⁵ 19.6 (c 1.66, CD₃OD) ¹H NMR (500 MHz, CD₃OD): δ 5.97 (d, 1H, J_{1,2}=1.4 Hz, H1), 5.34-5.29 (m, 4H, H2′, H2″, H2′″, H2″″), 5.26 (s, 1H, H2), 5.12 (s, 1H, H1″″), 5.09 (s, 3H, H1′, H1″, H1′″), 3.81-3.72 (m, 5H, H5, H5′, H5″, H5′″, H5″″), 3.57-3.35 (m, 4H, H3, H4′, H4″, H4′″), 3.51-3.44 (m, 5H, H4, H4″″, H3′, H3″, H3′″), 3.38 (2×s, 3H, OCH₃), 3.37 (s, 6H, OCH₃), 3.36 (s, 3H, OCH₃×2), 3.37-3.31 (m, 1H, H3″″), 2.12, 2.12, 2.10, 2.08, 2.07 (4×s, 12H, Ac), 2.06 (s, 6H, Ac×2), 1.35-1.25 (m, 15H, H6, H6′, H6″, H6′″, H6″″). ¹³C NMR (125 MHz, CD₃OD): δ 170.1 (×2), 170.1, 170.1, 170.0, 168.5 (C═O), 99.6 (C1″″), 99.6 (C1″), 99.5 (C1′), 99.4 (C1′″), 91.1 (C1), 80.1 (C3″), 80.0 (C3′), 80.0 (C3), 79.9 (C3′″), 79.4 (C3″″), 78.3 (C4′), 78.3 (C4″), 78.2 (C4′″), 78.1 (C4), 71.6 (C4″″), 69.2 (C5), 68.9 (C5″″), 68.0 (C2′″), 68.0 (C2″), 67.9 (C2″″), 67.8 (C2′), 67.8 (C5′), 67.7 (C5″), 67.5 (C5′″), 66.8 (C2), 57.6, 57.3, 57.3, 57.3, 57.2 (5×OCH₃), 21.1 (×2), 21.1, 21.0 (×2), 20.9 (6×Ac), 18.3 (C6), 18.2 (C6′, C6″, C6′″), 18.1 (C6″″) LRMS m/z Calcd for C₄₇H₇₄NaO₂₇ [M+Na]⁺, 1093.4; found, 1093.8.

### 3-O-Methyl-α-D-rhamnopyranoside-(1→4)-3-O-methyl-D-rhamnopyranose (disaccharide)

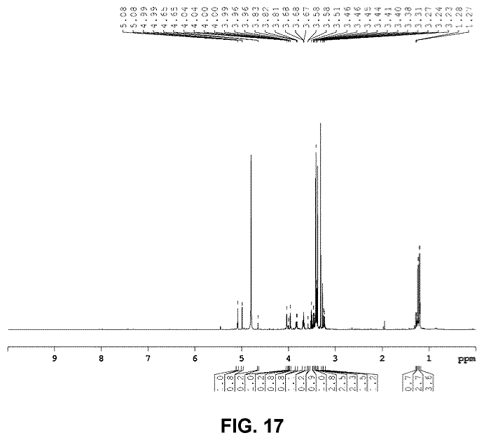

Compound 18 di (18 mg, 38.7 μmol) was dissolved in MeOH (5 mL) and sodium metal (1 mg) was added to the reaction mixture. After 16 h, rexin-H⁺ was added to the mixture until a pH of 5 was obtained. The mixture was then filtered and evaporated to give compound disaccharide without further purification (13.1 mg, 38.7 μmol, 100% yield). Rf=0.5 (MeOH/CH₂Cl₂, 1/5) ¹H NMR (600 MHz, CD₃OD): δ 5.08 (d, 1H, J_{1′,2}=1.8 Hz, H1′), 4.99 (d, 0.8H, J_{1α,2}=1.7 Hz, H1α), 4.65 (d, 0.2H, J_{1β,2}=0.9 Hz, H1β), 4.04 (dd, 1H, J_{2′,1′}=1.9 Hz, J_{2′,3′}=3.1 Hz, H2′), 3.99 (dd, 0.2H, J_{2β,1β}=1.1 Hz, J_{2β,3β}3.9 Hz, H2β), 3.96 (dd, 0.8H, J_{2α,1α}=1.9 Hz, J_{2α,3α}=2.9 Hz, H2α), 3.82 (m, 0.8H, H5α), 3.68 (m, 1H, H5′), 3.51 (dd, 0.8H, J_{4α,5α}=3.0 Hz, J_{4α,3α}=9.3 Hz, H4α), 3.48 (m, 0.2H, H4β) 3.46 (dd, 0.8H, J_{3α, 2α}=3.0 Hz, J_{3α, 4α}=9.3 Hz, H3α), 3.42-3.40 (m, 4H, H4′ and OCH₃′), 3.38 (s, 3H, OCH₃), 3.31 (m, 0.2H, H5β), 3.24 (dd, 1H, J_{3′,2′}=3.2 Hz, J_{3′,4′}=9.4 Hz, H3′), 3.23 (m, 0.2H, H3β), 1.34 (d, 0.6H, J_{6β,5β}=6.0 Hz, H6β) 1.24 (d, 2.4H, J_{6α,5α}=6.3 Hz, H6α), 1.20 (d, 3H, J_{6′,5′}=6.4 Hz, H6′). ¹³C NMR (150 MHz, CD₃OD): δ 103.2 (C1′), 95.5 (C1α), 95.4 (C1β), 85.5 (C3β), 83.0 (C3α), 82.0 (C3′), 79.8 (C4α), 79.2 (C4β), 72.6 (C4′), 72.0 (C5β), 70.5 (C5′), 68.5 (C2α), 68.4 (C2β and C2′), 67.8 (C5α), 57.3 (OCH₃′), 56.7 (OCH₃α), 56.5 (OCH₃β), 18.8 (C6α and C6β), 17.9 (C6′). J_{C1′, H1′α}=173 Hz, J_{C1, H1α}=172 Hz, J_{C1, H1β}=165 Hz. NMR spectrum is shown in FIG. 17.

### 3-O-methyl-α-D-rhamnopyranoside-(1→4)-3-O-methyl-α-D-rhamnopyranoside-(1→4)-3-O-methyl-α-D-rhamnopyranose (trisaccharide)

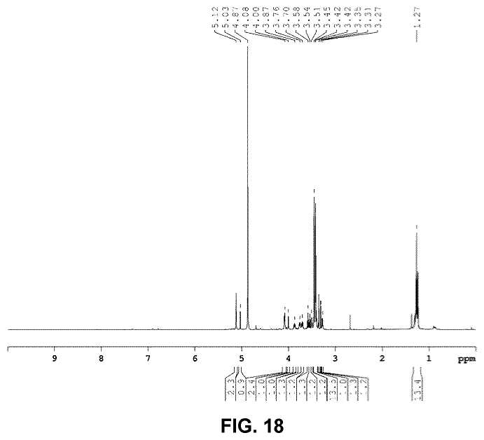

Compound 19 tri (25 mg, 37.5 μmol) was dissolved in 10 mL of anhydrous MeOH and the solution was stirred under an atmosphere of nitrogen gas. Sodium metal (10.0 mg, 0.44 mmol) was then added and the reaction was stirred until it reached completion by TLC. The solution was then neutralized with Rexin, filtered through celite, and evaporated to yield compound trisaccharide (18.1 mg, 36.3 μmol, 97%) as a clear solid. Rf=0.45 (MeOH/CH₂Cl₂, 1/4) [α]_{D}²⁵ 16.754 (c 1.4, MeOH) ¹H NMR (600 MHz, CD₃OD): δ 5.16 (m, 2H, H1′ and H1″), 5.07 (d, 0.8H, J_{1α,2}=1.6 Hz, H1α), 4.65 (d, 0.2H, J_{1β,2}=0.6 Hz, H1β), 4.12 (dd, 1H, J_{2′,1′}=2.2 Hz, J_{2′,3′}=2.6 Hz, H2′), 4.11 (dd, 1H, J_{2″,1″}=1.9 Hz, J_{2″,3″}=3.0 Hz, H2″), 4.07 (dd, 0.2H, J_{2β,1β}=0.9 Hz, J_{2β,3β}=2.9 Hz, H2β), 4.04 (dd, 1H, J_{2α,1α}=2.0 Hz, J_{2α,3α}=2.8 Hz, H2α), 3.90 (m, 0.8H, H5α), 3.80 (m, 1H, H5′) 3.74 (m, 1H, H5″), 3.62 (dd, 1H, J_{4α,5α}=J_{4α,3α}=9.2 Hz, H4′), 3.58 (dd, 0.8H, J_{4α,5α}=J_{4α,3α}=9.3 Hz, H4α), 3.56-3.49 (m, 1H, H3α and H4β), 3.45 (m, 1H, H3′), 3.49-3.48 (m, 4H, OCH₃″, H4″), 3.46 (s, 3H, OCH₃′), 3.45 (s, 3H, OCH₃), 3.39 (m, 0.2H, H5β), 3.33-3.29 (dd, 1.2H, J_{3″,2″}=3.0 Hz, J_{3″,4″}=9.5 Hz, H3″ and H3β), 1.35 (d, 0.6H, J_{6β,5β}=6.0 Hz, H6β), 1.32 (d, 3H, J_{6′,5′}=6.4 Hz, H6′), 1.29 (d, 2.4H, J_{6α,5α}=6.3 Hz, H6α), 1.28 (d, 3H, J_{6″,5″}=6.2 Hz, H6″). ¹³C NMR (150 MHz, CD₃OD): δ 103.2 (C1″), 103.2 (C1′), 95.5 (C1α), 95.4 (C1β), 85.4 (C3β), 83.1 (C3′), 82.9 (C3α), 82.0 (C3″), 80.0 (C4α), 79.4 (C4β), 79.3 (C4′), 72.6 (C4″), 71.9 (C5β), 70.5 (C5″), 69.0 (C5′), 68.6 (C2β), 68.5 (C2α), 68.3 (C2″), 68.0 (C2′), 67.7 (C5α), 57.3 (OCH₃″), 56.7 (OCH₃α), 57.6 (OCH₃′), 56.5 (OCH₃β), 18.8 (C6′ and C6β), 18.6 (C6α), 17.9 (C6″). J_{C1′, H1′α}=J_{C1″, H1″}=173 Hz, J_{C1α, H1α}=172 Hz, J_{C1β, H1β}=163 Hz J_{C1′, H1′α}=170 Hz LRMS m/z Calcd for C₂₁H₃₈NaO₁₃ [M+Na]⁺, 521.2; found, 521.2. NMR spectrum is shown in FIG. 18.

### 3-O-methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranose (tetrasaccharide)

Compound 19 tetra (22.6 mg, 26.0 μmol) was dissolved in 10 mL of anhydrous MeOH and the solution was stirred under an atmosphere of nitrogen gas. Sodium metal (10 mg, 0.44 mmol) was then added and the reaction was stirred until it reached completion by TLC. The solution was then neutralized with Rexin-H⁺ resin, filtered through celite, and evaporated to yield compound tetrasaccharide (15.9 mg, 24.1 μmol, 93%) as a clear solid. Rf=0.45 (MeOH/CH₂Cl₂, 1/4) [α]_{D}²⁵ 7.7 (c 0.63, MeOH) ¹H NMR (600 MHz, CD₃OD): δ 5.08 (m, 3H, H1′, H1″, H1′″), 5.00 (d, 0.8H, J_{1α,2}=1.8 Hz, H1α), 4.66 (d, 0.2H, J_{1β,2}=0.8 Hz, H1β), 4.05 (m, 2H, J_{2′,1′}=2.2 Hz, J_{2′,3′}=2.6 Hz, H2′ and H2″), 4.04 (dd, 1H, J_{2″,1″}=1.9 Hz, J_{2″,3″}=3.0 Hz, H2′″), 4.01 (dd, 0.2H, J_{2β,1β}=0.8 Hz, J_{2β,3β}=3.0 Hz, H2β), 3.96 (dd, 1H, J_{2α,1α}=1.9 Hz, J_{2α,3α}=2.8 Hz, H2α), 3.83 (m, 0.8H, H5α), 3.76-3.69 (m, 2H, H5′ and H5″), 3.67 (m, 1H, H5′″), 3.56-3.53 (m, 2H, H4′ and H4″), 3.51 (dd, 0.8H, J_{4α,5α}=J_{4α,3α}=9.3 Hz, H4α), 3.52-3.49 (m, 1H, H3α and H4β), 3.46 (dd, J_{3′,2′}=3.0 Hz, J_{3′,4′}=9.3 Hz, 1H, H3′), 3.42-3.40 (m, 4H, OCH₃′″, H4′″), 3.40-3.35 (s, 11H, OCH₃′, OCH₃″, OCH₃, H3″ and H3′), 3.33 (m, 0.2H, H5β), 3.25-3.22 (m, 1.2H, H3′″ and H3β), 1.28-1.21 (m, 9H, H6β, H6′, H6α and H6″), 1.20 (d, 3H, J_{6′″,5′″}=6.2 Hz, H6′″). ¹³C NMR (150 MHz, CD₃OD): δ 103.2 (C1′″), 103.0 (C1″), 103.0 (C1′) 95.5 (C1α), 95.4 (C1β), 85.4 (C3β), 83.0 (C3′), 83.0 (C3″) 82.9 (C3α), 82.0 (C3′″), 80.0 (C4α), 79.7 (C4β), 79.4 (C4′), 79.2 (C4″), 72.6 (C4′″), 71.9 (C5β), 70.5 (C5′″), 69.0 (C5′), 68.9 (C5″), 68.6 (C2β), 68.2 (C2α), 68.3 (C2′″), 68.0 (C2′ and C2″), 67.7 (C5α), 57.3 (OCH₃′″), 56.7 (OCH₃α), 56.7 (OCH₃′), 56.6 (OCH₃″), 56.5 (OCH₃β), 18.8 (C6α), 18.7 (C6β), 18.5 (C6′ and C6″), 17.9 (C6′″). J_{C1′, H1′}=J_{C1″, H1″}=J_{C1′″, H1′″}=173 Hz, J_{C1α, H1α}=174 Hz, J_{C1β, H1β}=162 Hz, LRMS m/z Calcd for C₂₈H₅₀NaO₁₇ [M+Na]⁺, 681.3; found, 680.9. NMR spectrum is shown in FIG. 19.

### 3-O-methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranose (pentasaccharide)

Compound 19 penta (33.1 mg, 30.9 μmol) was dissolved in 10.0 mL of anhydrous MeOH and the solution was stirred under an atmosphere of nitrogen gas. Sodium metal (10.0 mg, 0.44 mmol) was then added and the reaction was stirred until it reached completion by TLC. The solution was then neutralized with Rexin, filtered through celite, and evaporated to yield compound pentasaccharide (25.3 mg, 30.9 μmol, 100%) as a clear solid. Rf=0.45 (MeOH/CH₂Cl₂, 1/4) [α]_{D}²⁵ 12.6 (c 1.0, MeOH) ¹H NMR (600 MHz, CD₃OD): δ 5.12 (m, 4H, H1′, H1″, H1′″, H1″″), 5.03 (d, 0.8H, J_{1α,2}=1.8 Hz, H1α), 4.71 (s, 0.2H, H1β), 4.09 (m, 3H, H2′ and H2″, H2′″), 4.04 (dd, 1H, J_{2″″,1″″}=1.9 Hz, J_{2″″,3″″}=2.8 Hz, H2″″), 4.05 (dd, 0.2H, J_{2β,1β}=0.8 Hz, J_{2β,3β}=2.8 Hz, H2β), 4.00 (dd, 1H, J_{2α,1α}=2.0 Hz, J_{2α,3α}=2.6 Hz, H2α), 3.87 (m, 0.8H, H5α), 3.80-3.73 (m, 3H, H5′, H5″ and H5′″), 3.71 (dd, 1H, J_{5″″,4″″=}9.1 Hz, J_{5″″,6″″}=6.3 Hz, H5″″), 3.60-3.56 (m, 3H, H4′, H4″, H4′″), 3.55 (dd, 0.8H, J_{4α,5α}=J_{4α,3α}=9.3 Hz, H4α), 3.52-3.47 (m, 0.8H, H3α and H4β), 3.46-3.39 (m, 19.2H, OCH₃×5, H4″″, H3′, H3″ and H3′″, H5β), 3.29 (m, 1.2H, H3β and H3″″), 1.23-1.22 (m, 12H, H6β, H6′, H6α, H6″, H6′″), 1.24 (d, 3H, J_{6″″,5″″}=6.1 Hz, H6″″). ¹³C NMR (151 MHz, CD₃OD): δ 103.2 (C1″″), 103.1 (C1′″), 103.0 (C1″ and C1′), 95.5 (C1α), 95.3 (C1β), 85.3 (C3), 83.0 (C3′), 83.0 (C3″), 83.0 (C3′″), 82.9 (C3α), 82.0 (C3″″), 80.0 (C4α), 79.5 (C4β and C4′), 79.4 (C4″), 79.2 (C4′″), 72.6 (C4″″) 71.9 (C5β), 70.5 (C5″″), 69.0 (C5′), 69.0 (C5″), 68.9 (C5′″), 68.6 (C2β), 68.4 (C2α), 68.3 (C2″″), 68.0 (C2′, C2″ and C2′″), 67.7 (C5α), 57.3 (OCH₃″″), 56.7 (OCH₃α, OCH₃′, OCH₃″, OCH₃′″), 56.6 (OCH₃β), 18.8 (C6α), 18.6 (C6β, C6′, C6″ and C6′″), 17.9 (C6″″). J_{C1′, H1′}=J_{C1″, H1″}=J_{C1′″, H1′″}=J_{C1″″, H1″″}=173 Hz, J_{C1α,H1α}=170 Hz, J_{C1β,H1β}=160 Hz, LRMS m/z Calcd for C₃₅H₆₂NaO₂₁ [M+Na]⁺, 841.4; found, 841.3. NMR spectrum is shown in FIG. 20.

### 2-[N-(tert-Butoxycarbonyl)amino]ethyl 2-O-acetyl-4-O-benzyl-3-O-methyl-α-D-rhamnopyranoside (21)

Compound 16 (46.4 mg, 111 μmol) and N-boc-ethanolamine (19 μL, 123 μmol) were co-evaporated together with toluene (5×10 mL) and left to dry on high vacuum overnight. Anhydrous CH₂Cl₂ (5 mL) was added followed by 0.1 g of activated powdered 3 Å molecular sieves. The reaction was then cooled to −78° C. under an atmosphere of nitrogen gas and N-iodosuccinimide (33.7 mg, 150 μmol) was added followed by triflic acid (10 μL, 115 μmol). The reaction was stirred for 3 hours before reaching completion. The reaction was then filtered through a Buchner funnel and diluted with 10.0 mL of methylene chloride, washed with 10% Na₂S₂O₃ (2×10 mL) and a saturated sodium bicarbonate solution (1×10 mL). The organic layer was then dried with Na₂SO₄, filtered, and purified by flash chromatography (eluent: EtOAc/Hexane) to yield compound 21 (42.0 mg, 88 μmol, 80%) as clear oil. Rf=0.30 (EtOAc/Hexane, 3/7) [α]_{D}²⁵ 108.8 (c 22.5, CHCl₃) ¹H NMR alpha, (500 MHz, CDCl₃): δ 7.37-7.32 (m, 4H, Bn), 7.31-7.28 (m, 1H, Bn), 5.29 (dd, 1H, J_{2,1}=1.3 Hz, J_{2,3}=3.1 Hz, H2), 4.88 (d, 1H, J_{A,B}=10.9 Hz, CHA, OBn), 4.85 (br. s, 1H, NH), 4.71 (d, 1H, J_{1,2}=1.3 Hz, H1), 4.60 (d, 1H, J_{B,A}=10.9 Hz, CHB, OBn), 3.73-3.66 (m, 2H, H5 and CHA, CH₂CH₂NHBoc), 3.63 (dd, 1H, J_{3,2}=3.4 Hz, J_{3,4}=9.4 Hz, H3), 3.50-3.44 (m, 1H, CHB, CH₂CH₂NHBoc), 3.44 (s, 3H, CH₃O), 3.39-3.32 (m, 2H, H4 and CHA, CH₂CH₂NHBoc), 3.30-3.22 (m, 1H, CHB, CH₂CH₂NHBoc), 2.15 (s, 3H, Ac), 1.45 (s, 9H, C(CH₃)₃, Boc), 1.32 (d, 3H, J_{6,5}=6.3 Hz, H6). ¹³C NMR (125 MHz, CDCl₃): δ=170.5, 155.9 (C═O), 138.6, 128.5 (×2), 128.0 (×2), 127.8 (Bn), 97.8 (C1), 80.1 (C4), 79.9 (C3), 79.5 (CC(CH₃)₃), 75.4 (CH₂, Bn), 68.6 (C2), 67.8 (C5), 67.3 (CH₂CH₂NHBoc), 57.6 (OCH₃), 40.3 (CH₂CH₂NHBoc), 28.5 ((CH₃)₃, BOC), 21.1 (Ac), 18.0 (C6). HRMS m/z Calcd for C₂₃H₃₅NNaO₈ [M+Na]⁺, 476.2255; found, 476.2249.

### 2-[N-(tert-Butoxycarbonyl)amino]ethyl 2-O-acetyl-4-O-benzyl-3-O-methyl-α-D-rhamnopyranoside (22)

Compound 21 (46 mg, 101.5 μmol) was dissolved in MeOH (5 mL) and charged with palladium on carbon (40 mg, 0.38 mmol). The solution was then bubbled with a balloon of hydrogen gas for 5 minutes and continuously stirred under an atmosphere of hydrogen gas at room temperature. After 4 h, the reaction had reached completion and was therefore filtered through celite and evaporated to yield pure 22 (21.0 mg, 57.8 μmol, 62%) as a clear oil. Rf=0.35 (EtOAc/Hexane, 1/1), ¹H NMR (500 MHz, CDCl₃): δ 5.30 (dd, 1H, J_{2,1}=1.8 Hz, J_{2,3}=3.1 Hz, H2), 4.78 (br. s, 1H, NH), 4.74 (d, 1H, J_{1,2}=1.6 Hz, H1), 3.76-3.65 (m, 2H, H5 and CHA, CH₂CH₂NHBoc), 3.53-3.43 (m, 3H, H3, H4 and CHB, CH₂CH₂NHBoc), 3.41 (s, 3H, CH₃O), 3.39-3.26 (m, 2H, CH₂CH₂NHBoc), 2.34 (br. s, OH), 2.12 (s, 3H, Ac), 1.46 (s, 9H, C(CH₃)₃, Boc), 1.34 (d, 3H, J_{6,5}=6.2 Hz, H6). ¹³C NMR (125 MHz, CDCl₃): δ=170.3, 155.9 (C═O), 98.0 (C1), 79.4 (C4), 71.6 (C3), 68.3 (C2), 67.5 (C5), 67.2 (CH₂CH₂NHBoc), 57.3 (OCH₃), 40.2 (CH₂CH₂NHBoc), 28.4 ((CH₃)₃, BOC), 20.9 (Ac), 17.7 (C6). HRMS m/z Calcd for C₁₆H₂₉NNaO₈ [M+Na]⁺, 386.1791; found, 386.1784.

### 2-[N-(tert-butoxycarbonyl)amino]ethyl 2-O-Acetyl-4-O-benzyl-3-O-methyl-α-D-rhamnopyranoside-(1→4)-2-O-acetyl-3-O-methyl-α-D-rhamnopyranoside (23)

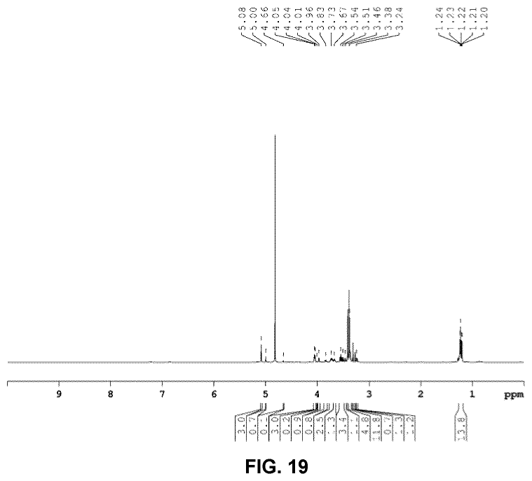

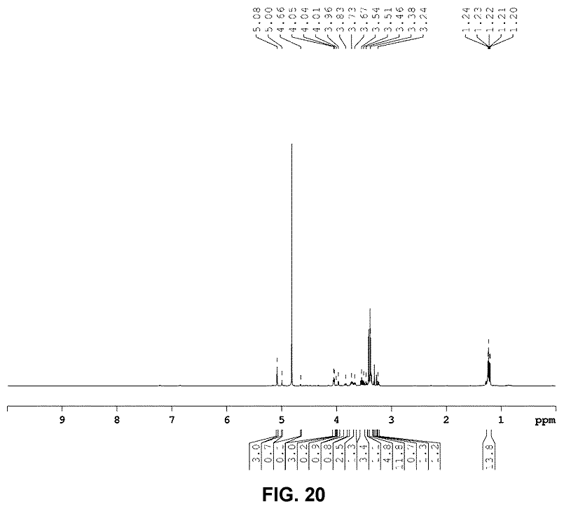

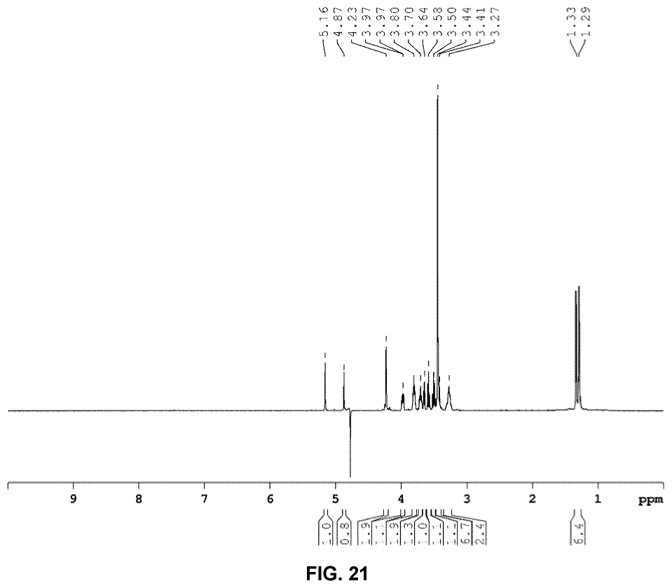

Compounds 22 (21.0 mg, 57.8 μmol) and 16 (28.9 mg, 69.4 μmol) were co-evaporated together with toluene (5×10 mL) and left to dry on high vacuum overnight. Anhydrous CH₂Cl₂ (5 mL) was added followed by 0.1 g of activated powdered 3 Å molecular sieves. The reaction was then cooled to −78° C. under an atmosphere of nitrogen gas and N-iodosuccinimide (19.1 mg, 84.9 μmol) was added followed by triflic acid (5.0 μL, 57.5 μmol). The reaction was stirred for 3 hours before reaching completion. The reaction was then filtered through a Buchner funnel and diluted with CH₂Cl₂ (10 mL), washed with 10% Na₂S₂O₃ (2×10 mL) and a saturated NaHCO₃ solution (1×10 mL). The organic layer was then dried with Na₂SO₄, filtered, and purified by flash chromatography (eluent: EtOAc/Hexane) to yield compound 23 (18 mg, 26.5 μmol, 47%) as clear oil. (Rf=0.65 EtOAc/Hexane 3/7). ¹H NMR alpha, (500 MHz, CDCl₃): δ 7.38-7.32 (m, 4H, Bn), 7.31-7.27 (m, 1H, Bn), 5.35 (m, 1H, H2′), 5.28 (m, 1H, H2), 5.12 (s, 1H, H1′), 4.88 (d, 1H, J_{A,B}=10.8 Hz, CHA, OBn), 4.83 (br. s, 1H, OH), 4.69 (s, 1H, H1), 4.62 (d, 1H, J_{B,A}=10.9 Hz, CHB, OBn), 3.85-3.77 (m, 1H, H5′), 3.75-3.68 (m, 1H, CHA, CH₂CH₂NHBoc), 3.66-3.61 (m, 1H, H5), 3.61-3.56 (m, 1H, H3′), 3.56-3.51 (m, 4H, H3 and H4), 3.51-3.42 (m, 4H, CHB, CH₂CH₂NHBoc and CH₃O), 3.41-3.34 (m, 4H, H4′ and CH₃O), 3.33-3.24 (m, 2H, CH₂CH₂NHBoc), 3.30 (bs, 1H, NH) 2.13, 2.10 (2×s, 6H, Ac×2), 1.46 (s, 9H, C(CH₃)₃, Boc), 1.33-1.27 (m, 6H, H6 and H6′). ¹³C NMR (125 MHz, CDCl₃): δ=170.4, 170.2 155.9 (C═O), 138.6, 128.4 (×2), 128.0 (×2), 127.8 (Bn), 99.4 (C1′), 97.8 (C1), 80.0 (C4′), 80.0 (C4), 79.9 (C3′), 79.6 (C(CH₃)₃), 78.3 (C3), 75.4 (CH₂, Bn), 68.7 (C2′), (C5′), 67.9 (C2), 67.3 (CH₂CH₂NHBoc), 67.1 (C5), 57.6 and 57.4 (OCH₃), 40.3 (CH₂CH₂NHBoc), 28.5 ((CH₃)₃, BOC), 21.2 and 21.1 (Ac), 18.2 and 17.9 (C6 and C6′). J_{C1′, H1′α}=171 Hz, J_{C1, H1}=168 Hz HRMS: m/z Calcd for C₃₂H₄₉NNaO₁₃ [M+Na]⁺, 678.3102; found, 678.3092. NMR spectrum is shown in FIG. 21.

### 2-Aminoethyl-3-O-methyl-α-D-rhamnopyranoside-(1→4)-3-O-methyl-α-D-rhamnopyranose (linker disaccharide)

Compound 23 (18 mg, 27 μmol) was dissolved in MeOH (2 mL) and PD/C (10 mg, 93 μmol) was added and the mixture was stirred over balloon pressure under a hydrogen atmosphere for 16 h. The compound was then diluted with MeOH (10 mL), filtered over celite and concentrated. The concentrate was then dissolved in MeOH (2 mL) and sodium solid (10 mg, 435 μmol) was added. The reaction was stirred under an atmosphere of nitrogen for 16 h, and rexin-H⁺ resin was added until a pH of 5 was reached. The mixture was filtrated and the filtrate was concentrated. The compound was then dissolved with DCM (2 mL) and TFA (160 μL) was added at 0° C. After 30 min at 0° C., the DCM and TFA mixture was evaporated to give linker disaccharide as a yellow oil. (10.3 mg, 27 μmol, 100% yield). (Rf=0.12, DCM/MeOH/Water/NH₄OH, 60/35/6/1) ¹H NMR (600 MHz, D₂O): δ 5.16 (d, 1H, J_{1,2}=1.6 Hz, H1′), 4.87 (d, 1H, J_{1,2}=1.7 Hz, H1), 4.23 (m, 2H, H2′ and H2) 3.97 (m, 1H, CHACH₂NH₂), 3.80 (m, 2H, H5′ and H5), 3.70 (m, 1H, CHBCH₂NH₂), 3.64 (dd, 1H, J_{3,2}=3.3 Hz, J_{3,4}=9.2 Hz, H3), 3.58 (dd, 1H, J_{4,3}=J_{4,5}=9.2 Hz, H4), 3.50 (dd, 1H, J_{4′,3′}=J_{4′,5′}=9.6 Hz, H4′), 3.44 (s, 3H, OCH₃), 3.44 (s, 3H, OCH₃), 3.41 (dd, 1H, J_{3′,2′}=3.3 Hz, J_{3′,4′}=9.7 Hz, H3′), 3.27 (m, 2H, CH₂NH₂), 1.33 (d, 3H, J_{6,5}=5.9 Hz, H6), 1.29 (d, 3H, J_{6′,5}′=6.4 Hz, H6′). ¹³C NMR (150 MHz, D₂O): δ 101.4 (C1′), 99.7 (C1), 80.5 (C3), 79.4 (C3′), 78.1 (C4), 70.6 (C4′), 69.5 (C5′), 67.2 (C5), 66.0 (C2′), 65.8 (C2), 63.4 (CH₂CH₂NH₂), 56.1 (2×OCH₃), 39.0 (CH₂NH₂), 17.3 (C6), 16.5 (C6′). J_{C1′, H1′α}=174 Hz, J_{C1, H1α}=173 Hz ESI-MS: m/z Calcd for C₁₆H₃₂NO₉ [M+H]⁺, 382.2072; found, 382.2071.

### Example 3A: Results & Discussion

Synthesis of 3-O-methyl D-rhamnose: D-rhamnose was first synthesized as described by Zunk et. al. and others [28-31] with slight modifications (Scheme 1, shown in FIG. 22). Acetylation of D-mannose followed by anomeric benzylation generated 1-O-Benzyl derivative 1 in 49% yield over 2 steps. Crude purification and treatment of 1 with sodium methoxide followed by iodination at C6 led to iodide 2 in excellent yields. Reduction of 2 with palladium hydroxide and hydrogen gas provided D-rhamnose derivative 3 in 100% yield. A doublet at 1.28 ppm in the ¹H NMR spectrum confirmed dehydroxylation at C6. The 2,3-O-acetonide protection of D-rhamnose derivative 3 followed by O-4 benzylation yielded to 4 in 90% yield over 2 steps. Diol 5 was obtained after acetonide deprotection. Subsequently, selective 3-O methylation with a dibutyltin oxide treatment and reaction with cesium fluoride and methyl iodide gave 6 in excellent yields (91%) over 2 steps [32]. A long range HMBC coupling between the O-methyl singlet and C3 confirmed the introduction of the methyl regioselectively. Global deprotection of intermediate 6 gave 3-O-methyl D-rhamnose 7 in 59% yield over 2 steps (HRMS m/z Calcd for C₇H₁₄NaO₅ [M+Na]⁺, 201.0739; found, 201.0734).

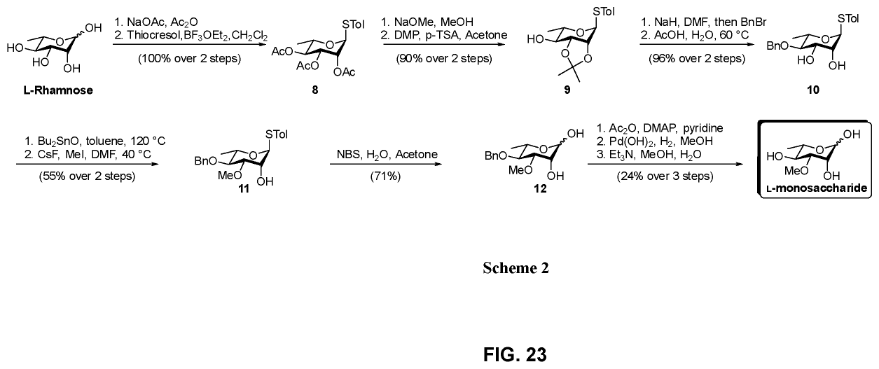

Synthesis of 3-O-methyl L-rhamnose: L-Rhamnose which is naturally abundant and inexpensive was converted to thioglycoside donor 8 in excellent yields after acetylation and reaction with thiocresol [31-36]. Intermediate 9 was obtained after deacetylation under Zemplén conditions and acetonide protection. Benzylation and acetonide deprotection yielded diol 10 in excellent yields. Selective methylation of 3-O using dibutyltin oxide followed by treatment with cesium fluoride and methyl iodide generated methylated derivative 11 in 55% yield over 2 steps. As previously described for intermediate 6, a strong long range signal between the O-methyl singlet and C3 confirmed the reaction regioselectivity. N-bromosuccinimide in acetone and water gave hemiacetal 12 in 710% yield. Acetylation followed by benzyl reduction and subsequent deacetylation gave 3-O-methyl L-rhamnose 13 in 24% yield over 3 steps (HRMS m/z Calcd for C₇H₁₄NaO₅ [M+Na]⁺, 201.0733; found, 201.0735) (Scheme 2, shown in FIG. 23).

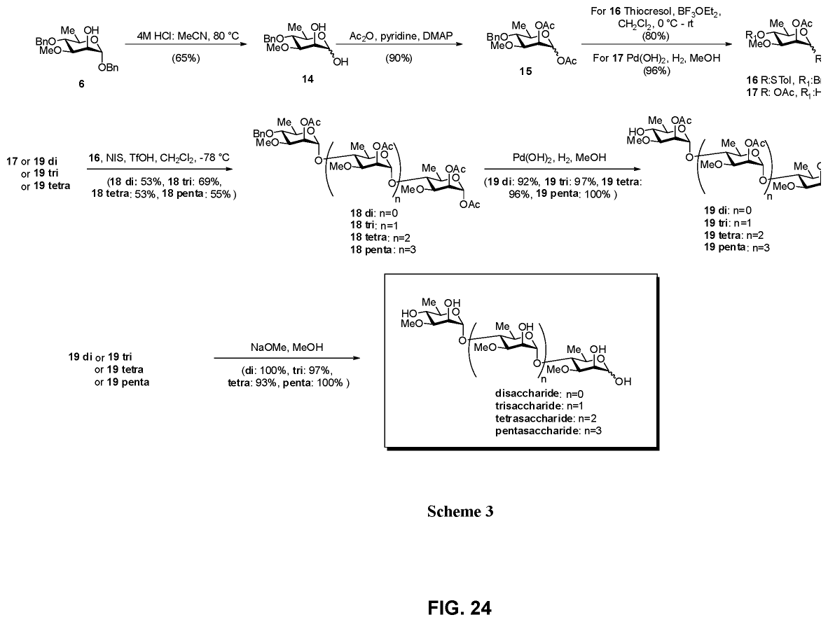

Synthesis of 3-O-methyl D-rhamnose oligosaccharides: With pure D-enantiomer intermediate 6 in hand, anomeric deprotection of 6 with an HCl, acetonitrile mixture generated hemiacetal 14, in 65% yield. 14 was then acetylated and treated with thiocresol and BF₃OEt₂ to produce donor 16 in good yields. Acceptor 17 was then generated after hydrogenation of benzylated intermediate 15 in excellent yields (96%). Subsequent glycosylation reaction between 16 and 17 activated with N-iodosuccinimide and triflic acid led to disaccharide 18 di in 53% yield (α only). A long range HMBC signal between H1′ and C4 additionally confirmed coupling. The resulting additional vicinal C1′, H1′ coupling constant (J_{C1′, H1′}=179 Hz) was indicative of an α-configuration and consistent with prior literature data [34]. The disaccharide acceptor 19 di was formed after benzyl deprotection as shown by the absence of signal in the ¹H NMR spectrum benzyl region. Pentasaccharide 18 penta was ultimately obtained after repeated iterative glycosylation and hydrogenation reactions. Each iterative glycosylation yielded slightly lower amounts of the respective oligosaccharides, as shown in Scheme 3 (shown in FIG. 24). The benzyl removal remained consistently high, yielding (90-100%) regardless of the length of the oligosaccharide. Final deprotection under Zemplén conditions of 19 penta yielded to target pentasaccharide 1 in 100% yield and 1.0% overall yield from D-mannose. Targets 19 di, 19 tri, and 19 tetra were also deprotected under Zemplén conditions to afford compounds 20 di, 20 tri and 20 tetra in excellent yields.

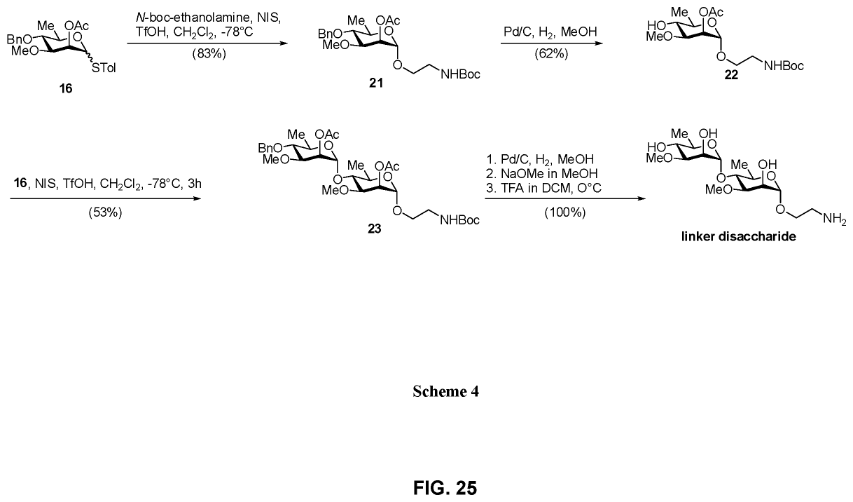

2-Aminoethyl handle glycosylation: To conjugate oligosaccharides to a protein, for the production of a conjugate vaccine, a common approach is to glycosylate the reducing end of an oligosaccharide with a handle [37, 38]. Glycosylation of intermediate 16 with N-boc ethanolamine gave intermediate 21 in good yields (Scheme 4, as shown in FIG. 25). A long range HMBC signal between H1 and the adjacent linker CH₂ additionally confirmed coupling. The resulting vicinal C1, H1 coupling constant was consistent of an α-configuration. [34] Benzyl deprotection with Pd/C yielded compound 22 in 62% yield; subsequent glycosylation of compound 22 with 16 generated 23 in 53% yield. Global deprotection of 23 produced the final target 24 in 100% yield.

### Example 4: Pseudomonas aeruginosa Methylated Rhamnan Synthetic Pentasaccharide Conjugation with HSA (without Handles or Linkers)

1.25 mg of HSA in water had 5 mg of methylated rhamnan synthetic pentasaccharide produced according to Example 3 (without handle or linker) in 300 μl of 20% methanol added to it. It was then left at room temperature for 1 h and lyophilized. Lyophilized material was immediately dissolved in 300 μl of 0.2M sodium borate containing 0.5 M sodium sulfate and 15 mg/ml of sodium cyanoborohydride and left for 72 h at 55° C. The sample was converted to water (spun 3× with water) using a Millipore ultra-15 30K MWCO spin column. An aliquot was checked by MALDI and SDS-PAGE, the remainder was lyophilized (1.1 mg) and stored at −20° C. until used to immunise mice. Six Balb/C mice were immunised via a prime and two boost schedule of the glycoconjugate with adjuvant. Sera following the second boost were examined in ELISA for cross-reactivity against LPS and whole cells from P. aeruginosa.

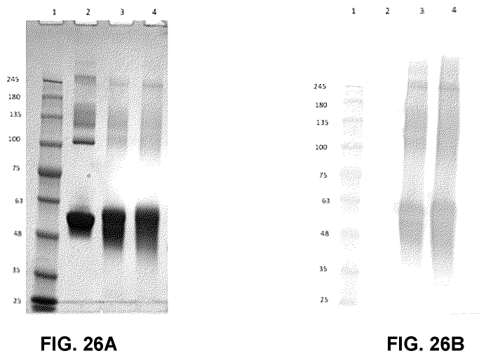

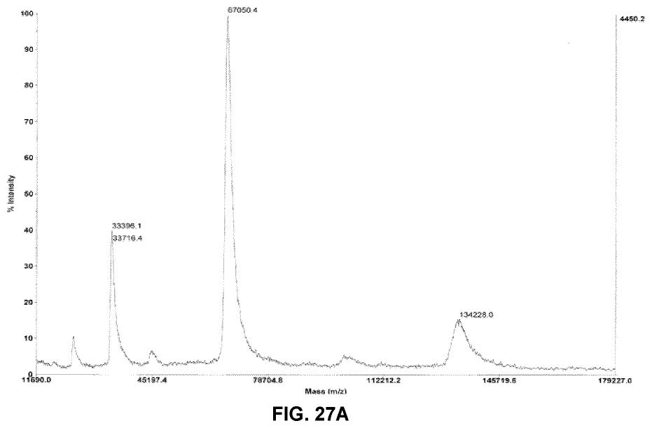

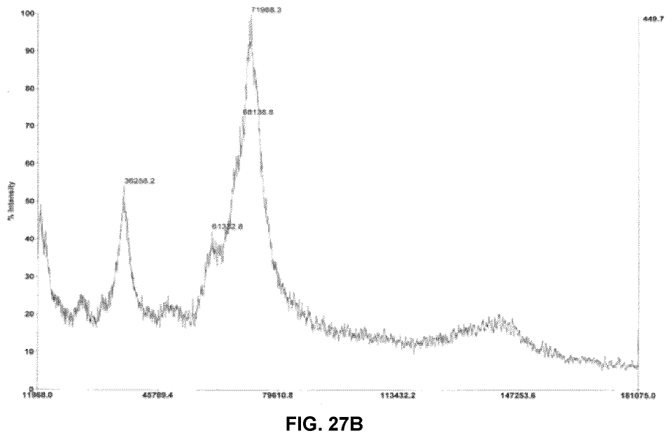

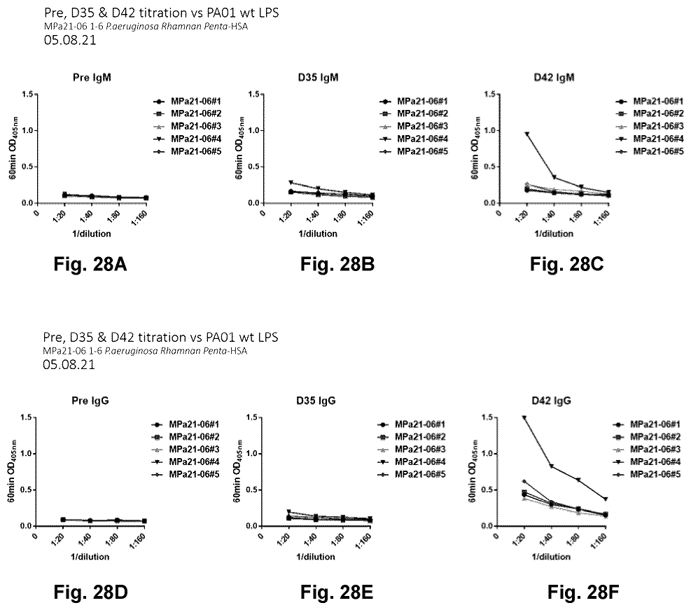

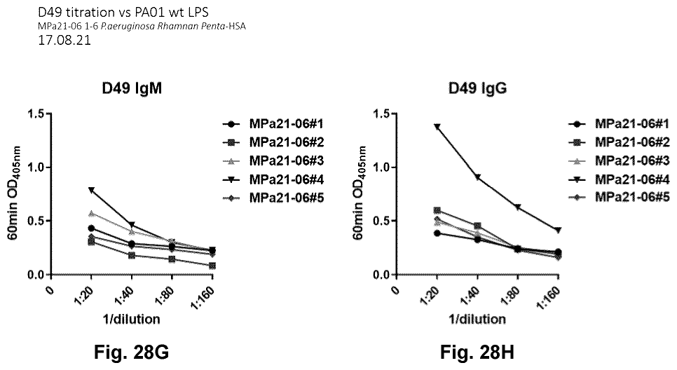

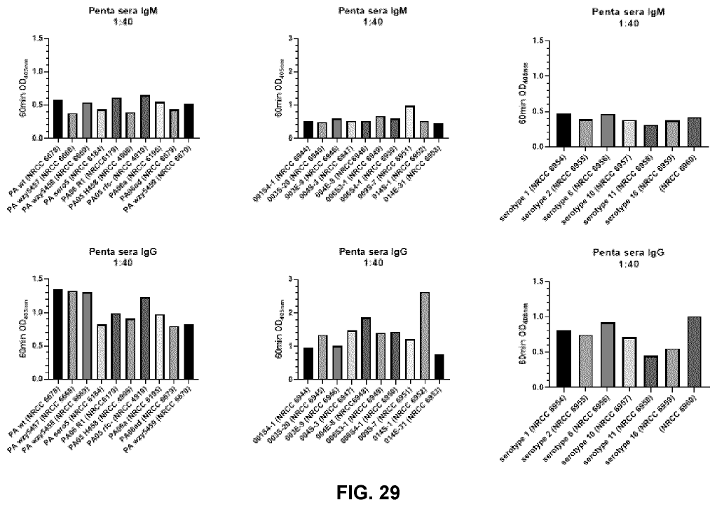

SDS-PAGE illustrated conjugation (FIGS. 26A and 26B) by virtue of the change in migration of the HSA molecule versus the conjugate. MALDI analysis indicated ˜6 pentasaccharides had been conjugated to HSA (FIG. 27) by virtue in the mass increase of ˜5 kDa as each pentasaccharide unit is 872 amu. ELISA analyses indicated that the derived sera recognised LPS (FIGS. 28A to 28F) and killed whole cells (FIG. 29) from P. aeruginosa, highlighting the ability of glycoconjugates of the synthetic pentasaccharide to achieve the required response to recognise the target P. aeruginosa epitope as elaborated on whole cells.

### Example 5: Synthesis of 3-O-Methyl Tri-, Tetra-, and Penta-Rhamnose Oligosaccharides with a Handle and Optionally a Linker

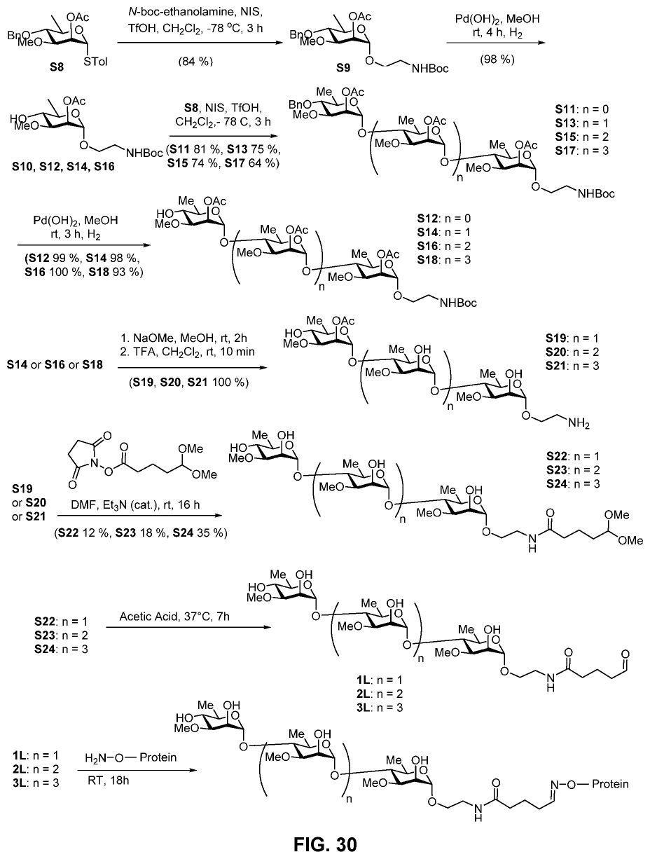

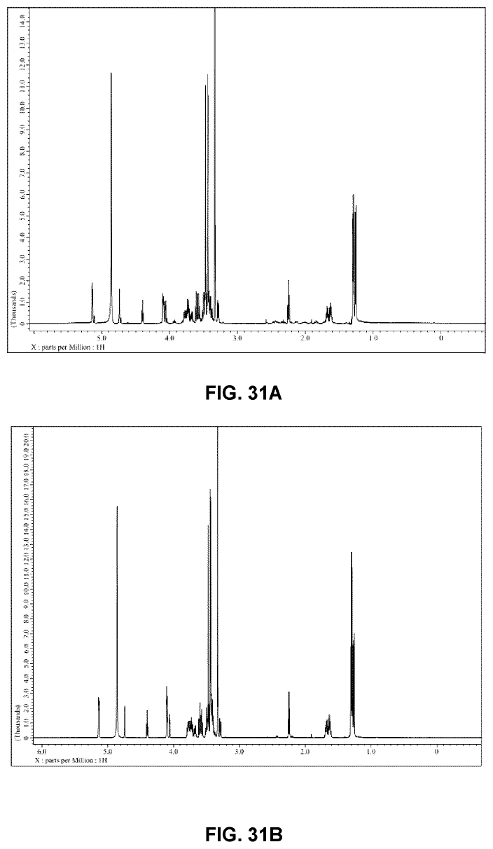

Reference is made to FIG. 30. An aminoethyl handle protected with Boc was attached to thiotoluene intermediate S8 (same as 16 above) in 84% yield. Then, removal of the benzyl protecting group under hydrogenation conditions gave donor S10 (same as 22 above) in excellent yields. Iterative 1,4 glycosylations with thiotoluene protected S8 (same as 16 above) followed by 4-O-benzyl deprotections generated S11 (same as 23 above), S13, S15, and S17. Quantitative 4-O-Benzyl deprotections of S13, S15, and S17 followed by deacetylation using sodium methoxide and Boc deprotection with TFA yielded S19 (same as 24 above), S20 and S21. Without further purification, these compounds were conjugated with a bivalent linker containing an activated N-hydroxysuccinimide and an hemiacetal protected aldehyde after semi-preparative reverse phase purification (see Scheme 5, FIG. 30).

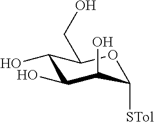

### p-Tolyl 1-thio-α-D-mannopyranoside (S1)

In a 2000 mL round bottom flask, acetylated mannose (142.5 g, 365.1 mmol) was dissolved in 800 mL of anhydrous CH₂C₁₂ and the solution was cooled to 0° C. under an atmosphere of N₂. Next, thiocresol (63.5 g, 511.0 mmol) was added, followed by boron trifluoride diethethyl etherate (63.1 mL, 511.0 mmol) and the solution was stirred at RT for 48 h at which point the TLC had shown completion. The reaction was then washed with water (2×200 mL) and NaHCO₃ (2×200 mL). The organic layer was isolated and dried with Na₂SO₄, filtered, and evaporated under reduced pressure. The crude product was then dissolved in 1000 mL of anhydrous MeOH, and cooled to 0° C. Sodium metal (0.873 g, 36.4 mmol) was then added and the reaction was stirred at RT for 30 h before reaching completion. The solution was neutralized with Dowex H⁺, filtered, and evaporated. The crude product was then dissolved in water (500 mL) and extracted with CH₂Cl₂ (3×150 mL). The water layer was then evaporated to afford S1 as a light green powder (96.86 g, 245 mmol, 67% over 2 steps). Rf=0.65 (MeOH/CH₂Cl₂, 15/85, V/V) [α]_{D}²⁵ 2.5 (c 0.16, MeOH) ¹H NMR (400 MHz, CD₃OD): δ 7.39 (d, 2H), 7.11 (d, 2H), 5.33 (s, 1H), 4.08-3.98 (m, 2H), 3.83-3.62 (m, 3H), 3.29 (s, 1H), 2.29 (s, 3H) ¹³C NMR (100 MHz, CD₃OD): δ 138.9, 133.5 (2C), 132.1, 130.8 (2C), 90.8, 75.5, 73.7, 73.1, 68.7, 62.6, 21.1 ESI-MS: m/z Calcd for C₁₃H₁₈O₅NaS [M+Na]⁺, 309.0767; found, 309.0769.

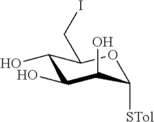

### p-Tolyl 6-iodo-1-thio-α-D-mannopyranoside (S2)

Compound S1 (96.86 g, 338.2 mmol) was dissolved in anhydrous THF (600 mL) and the solution was refluxed at 60-C under N₂. Next triphenylphosphine (133.05 g, 507.3 mmol), imidazole (46.04 g, 676.4 mmol), and I₂ (128.76 g, 507.3 mmol) was slowly added. The reaction was complete after 10 minutes when the purple color of iodine persisted. The solution was then cooled to RT and concentrated under reduced pressure. The crude mixture was dissolved in ethyl acetate (600 mL) and washed with 10% Na₂S₂O₃ (2×150 mL) and water (2×150 mL). The organic layer was then dried with Na₂SO₄, filtered, evaporated, and purified by flash chromatography (eluent: MeOH/CH₂Cl₂, 1/5, V/V) to yield compound S2 as a white solid (87.37 g, 220.5 mmol, 65%). Rf=0.65 (MeOH/CH₂Cl₂, 1/9, V/V) [α]_{D}²⁵−2.1 (c 0.041, MeOH) ¹H NMR (400 MHz, CD₃OD): δ 7.47 (d, 2H), 7.14 (d, 2H), 5.34 (s, 1H), 3.93 (dd, 1H), 3.67-3.52 (m, 3H), 3.31 (s, 1H), 2.32 (s, 3H) ¹³C NMR (100 MHz, CD₃OD): δ 138.9, 133.4 (2C), 132.2, 130.7 (2C), 91.0, 74.9, 73.8, 72.7, 72.6, 21.1, 6.0 ESI-MS: m/z Calcd for C₁₃H₁₇O₄INaS [M+Na]⁺, 418.9784; found, 418.9786.

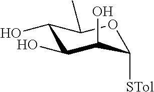

### p-Tolyl 1-thio-α-D-rhamnopyranoside (S3)

Compound S2 (1.76 g, 4.44 mmol) was dissolved in MeOH (30 mL) and the solution was charged with Pd(OH)₂ (0.39 g, 2.76 mmol) and the solution was bubbled with H2. Next, N,N-diisopropyl ethyl amine (1.8 mL, 10.3 mmol) was added and the solution was stirred under an atmosphere of H₂ for 6 h before reaching completion. The solution was then filtered through a pad of celite, evaporated, and purified by flash chromatography (eluent: MeOH/CH₂Cl₂, 1/5, V/V) to afford compound S3 as a clear oil (0.65 g, 2.40 mmol, 54%). Rf=0.50 (MeOH/CH₂C₁₂, 1/9, V/V) [α]_{D}²⁵ 0.91 (c 0.043, MeOH) ¹H NMR (400 MHz, CD₃OD): δ 7.35 (d, 2H), 7.14 (d, 2H), 5.29 (s, 1H), 4.10-4.00 (m, 2H), 3.64 (dd, 1H), 3.45 (dd, 1H), 3.31 (s, 1H), 2.31 (s, 3H), 1.26 (d, 3H) ¹³C NMR (100 MHz, CD₃OD): δ 138.8, 133.3 (2C), 132.2, 130.8 (2C), 90.6, 74.2, 73.8, 72.9, 72.6, 21.1, 17.8 ESI-MS: m/z Calcd for C₁₃H₁₈O₄NaS [M+Na]⁺, 293.0818; found, 293.0818.

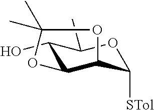

### p-Tolyl 2,3-O-isopropylidene-1-thio-α-D-rhamnopyranoside (S4)

Compound S3 (10.16 g, 37.6 mmol) was dissolved in 250 mL of anhydrous acetone. To this solution, 2,2-dimethoxypropane (8.29 mL, 67.6 mmol) and p-toluenesulfonic acid monohydrate (0.71 g, 0.376 mmol) were added and the reaction was stirred at RT for 3 h before reaching completion. The solution was evaporated under reduced pressure, dissolved in ethyl acetate (300 mL), and washed with saturated NaHCO₃ (2×100 mL). The combined organic layers were dried with Na₂SO₄, filtered, and purified by flash chromatography (eluent: EtOAc/Hexane, V/V) to afford compound S4 (10.68 g, 34.4 mmol, 92%) as a white solid. Spectral data agreed with literature values.¹

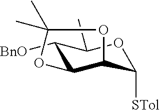

### p-Tolyl 4-O-benzyl-2,3-O-isopropylidene-1-thio-α-D-rhamnopyranoside (S5)

Compound S4 (10.68 g. 34.4 mmol) was dissolved in anhydrous DMF (150 mL) and the solution was cooled to 0° C. Next, NaH (2.06 g, 51.6 mmol) was added portion-wise and the solution was stirred for 30 mins. Benzyl bromide (4.91 mL. 41.3 mmol) was added dropwise and the solution was stirred at RT for 2 h before reaching completion. The reaction was quenched by the addition of Et₃N (10 mL) and was then poured into a saturated, cold solution of NH₄Cl (400 mL). The solution was stirred at 0° C. for 1 h, then filtered. The solids were collected and dried under high vacuum to yield S5 (13.43 g, 33.5 mmol, 98%) as a beige solid. Spectral data agreed with literature values.²

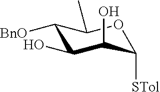

### p-Tolyl 4-O-benzyl-1-thio-α-D-rhamnopyranoside (S6)

Compound S5 (1.09 g, 2.72 mmol) was dissolved in an 80% (v/v) solution of AcOH (10 mL) and stirred overnight at RT. The solution was then heated to 55° C. in an oil bath for 3 h to drive the reaction to completion. The solution was then evaporated under reduced pressure and coevaporated with toluene (4×30 mL) to afford S6 (909 mg, 2.52 mmol, 92%) as a clear oil. Spectral data agreed with literature values.²

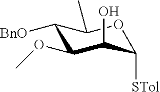

### p-Tolyl 4-O-benzyl-3-O-methyl-1-thio-α-D-rhamnopyranoside (S7)

Compound S6 (14.3 g, 39.7 mmol) was coevapporated with toluene (3×100 mL) and dried on high vacuum overnight. S6 was then dissolved in anhydrous toluene (400 mL), purged with N₂ gas and charged with dibultyltin (IV) oxide (11.85 g, 47.6 mmol). The reaction mixture was then stirred under reflux for 16 h, cooled to RT, and evaporated under reduced pressure. The crude tin acetal was left on high vacuum for 5 h, dissolved in anhydrous DMF (793 mL) and purged with N₂. Cesium fluoride (9.00 g, 59.6 mmol) and iodomethane (28.15 mL, 396.6 mmol) were added and the reaction was stirred with 40° C. for 16 h under N₂. The mixture was cooled to RT and diluted with EtOAc (350 mL), washed with water (5×150 mL), saturated NaHCO₃ (150 mL), and brine (150 mL). The combined organic layers were then dried with Na₂SO₄, evaporated and purified by flash chromatography (eluent: EtOAc/hexane, V/V) to afford S7 as a clear oil (10.87 g, 29.0 mmol, 73%). Rf=0.40 (EtOAc/Hexane, 3/7, V/V) [α]_{D}²⁵ 8.2 (c 0.38, CHCl₃) ¹H NMR (400 MHz, CD₃Cl): δ 7.40-7.27 (m, 8H), 7.11 (d, 2H), 5.47 (d, 1H), 4.86 (d, 1H). 4.64 (d, 1H), 4.30 (dd, 1H), 4.20 (dq, 1H), 3.58 (dd, 1H), 3.52 (s, 3H), 3.44 (dd, 1H), 2.32 (s, 3H), 2.28 (br. s, 1H), 1.30 (d, 3H) ¹³C NMR (100 MHz, CD₃Cl): δ 138.6. 137.8, 132.2 (2C), 130.4, 130.0 (2C), 128.6 (2C), 128.1 (2C), 127.9, 87.6, 82.1, 80.2, 75.4, 69.5, 68.6, 57.6, 21.2, 17.9 ESI-MS: m/z Calcd for C₂₁H₂₆O₄NaS [M+Na]⁺, 397.1444; found, 397.1444.

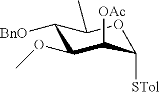

### p-Tolyl 2-O-acetyl-4-O-benzyl-3-O-methyl-1-thio-α-D-rhamnopyranoside (S8 (Same as 16 Above))

Compound S7 (10.85 g, 29.0 mmol) was dissolved in acetic anhydride (20 mL) and pyridine (20 mL) and the solution was stirred at RT for 16 h. The solution was then evaporated under reduced pressure and coevaporated with toluene (5×30 mL). The crude oil was then purified by flash chromatography (eluent: EtOAc/Hexane, V/V) to yield compound S8 (10.77 g, 25.9 mmol, 89%) as a clear oil. Spectral data agreed with literature values.³

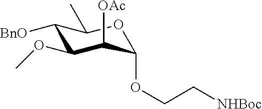

### 2-(tert-Butyloxycarbonylamido)ethyl 2-O-acetyl-4-O-benzyl-3-O-methyl-α-D-rhamnopyranoside (S9 (Same as 21 Above))

Compound S8 (46.4 mg, 0.11 mmol) and N-boc-ethanolamine were co-evaporated together with toluene (5×10 mL) and left to dry on high vacuum overnight. Anhydrous CH₂Cl₂ (5 mL) was added followed by 0.1 g of activated powdered 3 Å molecular sieves and the reaction was cooled to −78° C. under an atmosphere of N₂ gas. Next, N-iodosuccinimide (33.7 mg, 0.12 mmol) was added followed by triflic acid (10.0 μL, 0.12 mmol) and the reaction was stirred for 3 h before reaching completion. The mixture was filtered through a Buchner funnel, diluted with CH₂Cl₂ (10 mL), and washed with 10% Na₂S₂O₃ (2×10 mL) and saturated NaHCO₃ (10 mL). The organic layer was then dried with Na₂SO₄, filtered, and purified by flash chromatography (eluent: EtOAc/Hexane, V/V) to yield compound S9 as a clear oil (42.0 mg, 0.093 mmol, 84%). Rf=0.55 (EtOAc/Hexane, 1/1, V/V) [α]_{D}²⁵ 108.8 (c 2.3, CHCl₃) ¹H NMR (500 MHz, CDCl₃): δ 7.45-7.36 (m, 5H), 4.89 (d, 1H), 4.84 (br. s, 1H), 4.70 (s, 1H), 4.61 (d, 1H), 3.72-3.65 (m, 2H), 3.63 (dd, 1H), 3.49-3.44 (m, 1H), 3.42 (s, 3H), 3.36-3.30 (m, 1H), 3.35 (dd, 1H), 3.30-3.21 (m, 1H), 2.14 (s, 3H), 1.44 (s, 9H), 1.31 (d, 3H) ¹³C NMR (150 MHz, CDCl₃): δ 170.5, 155.9, 138.6, 128.5 (2C), 128.0 (2C), 127.8, 97.8, 80.1, 79.9, 75.4, 68.6, 67.8, 67.3, 57.6, 40.3, 28.5, 21.1, 18.0 ESI-MS: m/z Calcd for C₂₃H₃₅NO₈H [M+H]⁺, 454.2435; found, 454.2435.

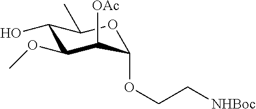

### 2-(tert-Butyloxycarbonylamido)ethyl 2-O-acetyl-3-O-methyl-α-D-rhamnopyranoside (S10 (Same as 22 Above))

Palladium hydroxide on carbon (1.00 g, 7.00 mmol) was added to MeOH (20 mL) and the solution was bubbled with H2 for 30 minutes. Next, compound S9 (1.37 g, 3.02 mmol) was added and the solution was stirred under a continuous flow of H2 for 3 h at RT. The reaction was then filtered through a pad of celite and evaporated to afford S10 (1.07 g, 2.94 mmol, 98%) as a clear oil. Rf=0.35 (EtOAc/Hexane, 1/1, V/V) [α]_{D}²⁵ 9.6 (c 0.28, CHCl₃) ¹H NMR (500 MHz, CDCl₃/CD₃OD): δ 5.29 (s, 1H), 4.78 (br. s, 1H), 4.74 (s, 1H), 3.76-3.65 (m, 2H), 3.54-3.47 (m, 2H), 3.46 (dd, 1H), 3.41 (s, 3H), 3.38-3.36 (m, 2H), 2.34 (br. s, 1H), 2.11 (s, 3H), 1.46 (s, 9H), 1.33 (d, 3H) ¹H NMR (125 MHz, CDCl₃/CD₃OD): δ 170.4, 155.9, 98.0, 79.5, 71.6, 68.3, 67.5, 67.2, 57.3, 40.3, 28.4, 20.9, 17.7 ESI-MS: m/z Calcd for C₁₆H₂₉NO₈H [M+H]⁺, 364.1966; found, 364.1963.

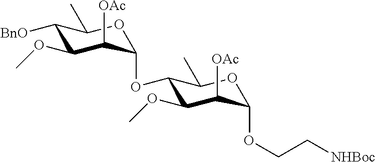

### 2-O-Acetyl-4-O-benzyl-3-O-methyl-α-D-rhamnopyranoside-(1→4)-2-(tert-butoxycarbonyl)amino]ethyl 2-O-acetyl-3-O-methyl-α-D-rhamnopyranoside (S11 (Same as 23 Above))

Compounds S10 (1.07 g, 2.93 mmol) and S8 (1.47 g, 3.53 mmol) were coevapporated together with toluene (5×20 mL) and left to dry on high vacuum overnight. Anhydrous methylene chloride (50 mL) was added followed by 0.500 g of activated powdered 3 Å molecular sieves. The reaction was then cooled to −78° C. under an atmosphere of N₂ and N-iodosuccinimide (1.13 g, 4.11 mmol) was added followed by triflic acid (0.50 mL, 5.66 mmol). The reaction was stirred for 3 h before reaching completion. The reaction was then filtered through a Buchner funnel, diluted with CH₂Cl₂ (100 mL), washed with 10% Na₂S₂O₃ (2×100 mL) and saturated NaHCO₃ (100 mL). The organic layer was then dried with Na₂SO₄, filtered, and purified by flash chromatography (eluent: EtOAc/Hexane, V/V) to yield compound S11 (1.56 g, 2.38 mmol, 81%) as a clear oil. Rf=0.65 (EtOAc/Hexane, 1/1, V/V) [α]_{D}²⁵ 66.8 (c 1.0, CHCl₃) ¹H NMR (400 MHz, CDCl₃): δ 7.38-7.27 (m, 5H), 5.35 (dd, 1H), 5.28 (s, 1H), 5.12 (d, 1H), 4.89 (d, 1H), 4.82 (br. s, 1H), 4.69 (d, 1H), 4.61 (d, 1H), 3.86-3.77 (m, 1H), 3.75-3.66 (m, 1H), 3.66-3.55 (m, 1H), 3.55-3.45 (m, 3H), 3.44 (s, 3H), 3.39 (s, 3H), 3.39-3.33 (m, 2H), 3.33-3.23 (m, 1H), 2.13 (s, 3H), 2.10 (s, 3H), 1.46 (s, 9H), 1.34-1.28 (m, 6H) ¹³C NMR (125 MHz, CDCl₃): δ 170.4, 170.2, 155.9, 138.6, 128.4 (2C), 128.0 (2C), 127.8, 99.4, 97.8, 80.1, 80.0, 79.9, 78.3, 75.4, 68.7, 68.5, 67.9, 67.3, 67.1, 57.6, 57.4, 40.3, 28.5, 21.2, 21.0, 18.2, 17.9 ESI-MS: m/z Calcd for C₃₂H₄₉NNaO₁₃ [M+Na]⁺, 678.3096; found, 678.3092.

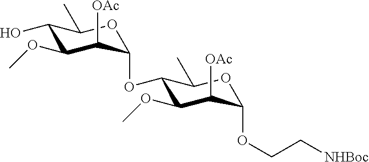

### 2-O-Acetyl-3-O-methyl-α-D-rhamnopyranoside-(1→4)-2-(tert-butoxycarbonyl)amino]ethyl 2-O-acetyl-3-O-methyl-α-D-rhamnopyranoside (S12)

Palladium hydroxide on carbon (1.20 g, 8.55 mmol) was added to MeOH (50 mL) and the solution was bubbled with H2 for 30 minutes. Next, compound S11 (1.71 g, 2.61 mmol) was added and the solution was stirred under a continuous flow of H2 for 3 h at RT. The reaction was then filtered through a pad of celite and evaporated to afford S12 (1.47 g, 2.59 mmol, 99%) as a clear oil. Rf=0.20 (EtOAc/Hexane, 1/1, V/V) [α]_{D}²⁵ 1.07 (c 0.33, CHCl₃) ¹H NMR (400 MHz, CDCl₃): δ 5.36 (dd, 1H), 5.31-5.26 (m, 1H), 5.14 (s, 1H), 4.82 (br. s, 1H), 4.70 (s, 1H), 3.84-3.75 (m, 1H), 3.75-3.62 (m, 1H), 3.58-3.44 (m, 4H), 3.43-3.34 (m, 8H), 3.34-3.21 (m, 1H), 2.38 (br. s, 1H), 2.12-2.08 (m, 6H), 1.46 (s, 9H), 1.35-1.29 (m, 6H) ¹³C NMR (100 MHz, CDCl₃): δ 170.5, 170.2, 155.9, 99.8, 97.9, 80.1, 79.7, 79.5, 78.4, 77.4, 71.7, 69.0, 67.9, 67.5, 67.4, 67.2, 57.5, 57.3, 40.4, 28.6, 21.2, 21.1, 18.3, 17.7 ESI-MS: m/z Calcd for C₃₂H₄₃O₁₃NH [M+H]⁺, 566.2807; found, 566.2788.

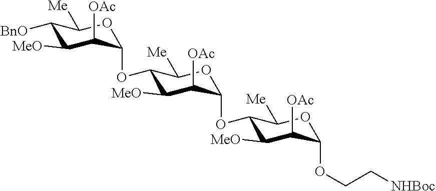

### 2-O-Acetyl-4-O-benzyl-3-O-methyl-α-D-rhamnopyranoside-(1→4)-2-O-acetyl-3-O-methyl-α-D-rhamnopyranoside-(1→4)-2-(tert-butoxycarbonyl)amino]ethyl 2-O-acetyl-3-O-methyl-α-D-rhamnopyranoside (S13)

Compounds S12 (1.46 g, 2.57 mmol) and S8 (1.29 g, 3.10 mmol) were coevapporated together with toluene (5×20 mL) and left to dry on high vacuum overnight. Anhydrous methylene chloride (50 mL) was added followed by 0.400 g of activated powdered 3 Å molecular sieves. The reaction was then cooled to −78° C. under an atmosphere of N₂ and N-iodosuccinimide (990.0 mg, 3.60 mmol) was added followed by triflic acid (0.40 mL, 4.52 mmol). The reaction was stirred for 3 h before reaching completion. The reaction was then filtered through a Buchner funnel, diluted with CH₂Cl₂ (100 mL), washed with 10% Na₂S₂O₃ (2×100 mL) and saturated NaHCO₃ (100 mL). The organic layer was then dried with Na₂SO₄, filtered, and purified by flash chromatography (eluent: EtOAc/Hexane, V/V) to yield compound S13 (1.65 g, 1.92 mmol, 75%) as a clear oil. Rf=0.45 (EtOAc/Hexane, 1/1, V/V) [α]_{D}²⁵ 2.0 (c 0.46, CHCl₃) ¹H NMR (600 MHz, CDCl₃): δ 7.37-7.26 (m, 5H), 5.35 (s, 1H), 5.33 (s, 1H), 5.27 (s, 1H), 5.10 (s, 1H), 5.09 (s, 1H), 4.88 (d, 1H), 4.83 (br. s, 1H), 4.61 (d, 1H), 3.85-3.71 (m, 1H), 3.77-3.65 (m, 3H), 3.59 (dd, 1H), 3.56-3.45 (m, 5H), 3.44 (s, 3H), 3.40 (s, 3H), 3.38 (s, 3H), 3.38-3.33 (m, 2H), 3.32-3.26 (m, 1H), 2.13 (s, 3H), 2.10 (s, 3H), 2.08 (s, 3H), 1.46 (s, 9H), 1.33-1.30 (m, 6H), 1.28 (d, 3H) ¹³C NMR (150 MHz, CDCl₃): δ 170.4, 170.2, 170.1, 155.9, 138.6, 128.5 (2C), 128.1 (2C), 127.8, 99.5, 97.9, 80.1, 80.0, 80.0, 78.7, 78.4, 75.5, 68.7, 68.5, 68.0, 67.9, 67.8, 67.3, 67.1, 57.6, 57.5, 57.3, 40.4, 28.5, 21.2, 21.1, 18.3, 18.1, 17.9 ESI-MS: m/z Calcd for C₄₁H₆₃O₁₈NNa [M+Na]⁺, 880.3937; found, 880.3943.

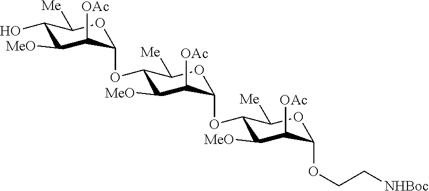

### 2-O-Acetyl-3-O-methyl-α-D-rhamnopyranoside-(1→4)-2-O-acetyl-3-O-methyl-α-D-rhamnopyranoside-(1→4)-2-(tert-butoxycarbonyl)amino]ethyl 2-O-acetyl-3-O-methyl-α-D-rhamnopyranoside (S14)

Palladium hydroxide on carbon (807.0 mg, 5.67 mmol) was added to MeOH (50 mL) and the solution was bubbled with H2 for 30 minutes. Next, compound S13 (1.65 g, 1.92 mmol) was added and the solution was stirred under a continuous flow of H₂ for 3 h at RT. The reaction was then filtered through a pad of celite and evaporated to afford S13 (1.57 g, 1.89 mmol, 98%) as a clear oil. Rf=0.20 (EtOAc/Hexane, 1/1, V/V) [α]_{D}²⁵ 2.3 (c 0.51, CHCl₃) ¹H NMR (600 MHz, CD₃OD): δ 5.40 (dd, 1H), 5.36 (s, 1H), 5.32 (s, 1H), 5.10 (d, 1H), 5.06 (d, 1H), 4.75 (s, 1H), 3.88-3.82 (m, 1H), 3.81-3.75 (m, 2H), 3.75-3.71 (m, 1H), 3.69 (dd, 1H), 3.57 (dd, 1H), 3.53-3.45 (m, 3H), 3.43 (s, 3H), 3.40 (s, 3H), 3.39 (s, 3H), 3.35-3.32 (m, 4H), 3.32-3.24 (m, 2H), 2.13-2.11 (m, 6H), 2.10 (s, 3H), 1.50 (s, 9H), 1.33-1.30 (m, 6H), 1.29 (d, 3H) ¹³C NMR (150 MHz, CD₃OD): δ 171.7 (2C), 171.6, 158.5, 101.0, 100.8, 98.7, 81.5, 81.3, 80.8, 80.4, 79.6, 72.9, 72.9, 70.8, 69.6, 69.3, 69.2, 69.0, 68.0, 67.5, 58.0, 57.7, 41.1, 28.9, 20.7, 18.6, 18.5, 17.9 ESI-MS: m/z Calcd for C₃₄H₅₇O₁₈NNa [M+Na]⁺, 790.3468; found, 790.3462.

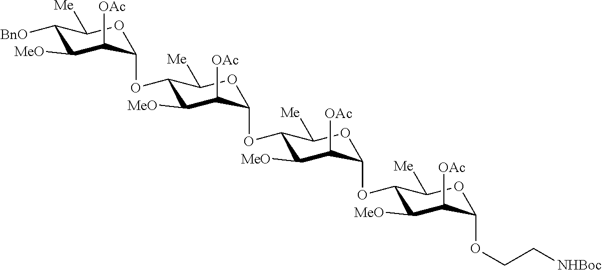

### 2-O-Acetyl-4-O-benzyl-3-O-methyl-α-D-rhamnopyranoside-(1→4)-2-O-acetyl-3-O-methyl-α-D-rhamnopyranoside-(1→4)-2-O-acetyl-3-O-methyl-α-D-rhamnopyranoside-(1→4)-2-(tert-butoxycarbonyl)amino]ethyl 2-O-acetyl-3-O-methyl-α-D-rhamnopyranoside (S15)

Compounds S14 (1.45 g, 1.89 mmol) and S8 (943.9 mg, 2.27 mmol) were coevapporated together with toluene (5×20 mL) and left to dry on high vacuum overnight. Anhydrous methylene chloride (50 mL) was added followed by 0.400 g of activated powdered 3 Å molecular sieves. The reaction was then cooled to −78° C. under an atmosphere of N₂ and N-iodosuccinimide (727.0 mg, 2.64 mmol) was added followed by triflic acid (0.35 mL, 3.96 mmol). The reaction was stirred for 3 h before reaching completion. The reaction was then filtered through a Buchner funnel, diluted with CH₂Cl₂ (100 mL), washed with 10% Na₂S₂O₃ (2×100 mL) and saturated NaHCO₃ (100 mL). The organic layer was then dried with Na₂SO₄, filtered, and purified by flash chromatography (eluent: EtOAc/Hexane, V/V) to yield compound S15 (1.49 g, 1.41 mmol, 74%) as a clear oil. Rf=0.40 (EtOAc/Hexane, 1/1, V/V) [α]_{D}²⁵ 1.7 (c 0.31, CHCl₃) ¹H NMR (600 MHz, CDCl₃): δ 7.37-7.26 (m, 5H), 5.35 (s, 3H), 5.28 (s, 1H), 5.13-5.06 (m, 3H), 4.89 (d, 1H), 4.83 (br. s, 1H), 4.70 (s, 1H), 4.61 (d, 1H), 3.87-3.63 (m, 5H), 3.63-3.57 (m, 1H), 3.57-3.46 (m, 7H), 3.44 (s, 3H), 3.41-3.24 (m, 13H), 2.15-2.04 (m, 12H), 1.46 (s, 9H), 1.35-1.26 (m, 12H) ¹³C NMR (150 MHz, CDCl₃): δ 170.1, 170.3, 170.2, 170.1, 155.9, 138.6, 128.5 (2C), 128.1 (2C), 127.9, 99.6, 99.5, 97.9, 80.1, 80.0, 79.9, 79.7, 78.9, 78.4, 77.4, 75.5, 68.7, 68.5, 68.1, 68.0, 67.9, 67.8, 67.7, 67.3, 67.1, 40.4, 28.6, 21.3, 21.2, 18.4, 18.2, 18.1, 18.0 ESI-MS: m/z Calcd for C₅₀H₇₇NO₂₃H [M+H]⁺, 1060.4959; found, 1060.4977.

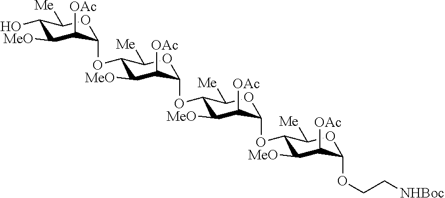

### 2-O-Acetyl-3-O-methyl-α-D-rhamnopyranoside-(1→4)-2-O-acetyl-3-O-methyl-α-D-rhamnopyranoside-(1→4)-2-O-Acetyl-3-O-methyl-α-D-rhamnopyranoside-(1→4)-2-(tert-butoxycarbonyl)amino]ethyl 2-O-acetyl-3-O-methyl-α-D-rhamnopyranoside (S16)

Palladium hydroxide on carbon (720.0 mg, 5.13 mmol) was added to MeOH (50 mL) and the solution was bubbled with H2 for 30 minutes. Next, compound S15 (1.42 g, 1.34 mmol) was added and the solution was stirred under a continuous flow of H₂ for 3 h at RT. The reaction was then filtered through a pad of celite and evaporated to afford S16 (1.30 g, 1.34 mmol, 100%) as a clear oil. Rf=0.15 (EtOAc/Hexane, 3/2, V/V) [α]_{D}²⁵ 3.4 (c 0.67, CHCl₃) ¹H NMR (600 MHz, CD₃OD): δ 5.42 (dd, 1H), 5.40 (dd, 1H), 5.38 (dd, 1H), 5.33 (dd, 1H), 5.10 (d, 1H), 5.08 (d, 1H), 5.06 (d, 1H), 4.75 (d, 1H), 3.90-3.82 (m, 2H), 3.82-3.76 (m, 2H), 3.76-3.72 (m, 1H), 3.72-3.66 (dd, 1H), 3.62-3.55 (m, 2H), 3.55-3.46 (m, 4H), 3.46-3.43 (s, 3H), 3.43-3.36 (m, 11H), 3.33-3.22 (m, 2H), 2.14-2.11 (m, 9H), 2.11 (s, 3H), 1.51 (s, 9H), 1.36-1.31 (m, 9H), 1.30 (d, 3H) ¹³C NMR (150 MHz, CD₃OD): δ 171.7 (2C), 171.6 (2C), 158.5, 101.0, 100.9, 100.8, 98.7, 81.4, 81.3, 81.2, 80.9, 80.3, 80.2, 80.0, 79.6, 72.9, 70.7, 69.6, 69.3, 69.2, 69.1, 69.0, 68.9, 68.0, 67.6, 58.0, 57.6, 41.1, 29.0, 20.7, 18.6, 18.5, 18.4, 17.9 ESI-MS: m/z Calcd for C₄₃H₇₁O₂₃NNa [M+Na]⁺, 992.4309; found, 992.4313.

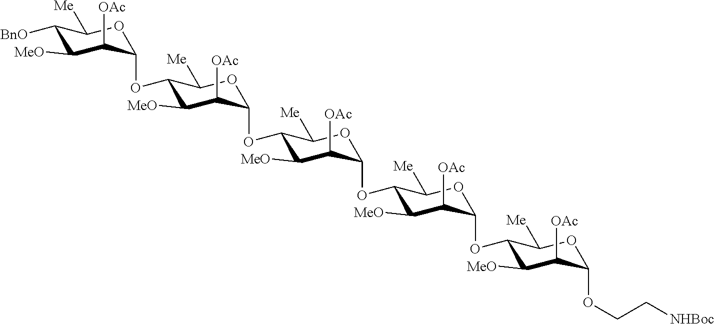

### 2-O-Acetyl-4-O-benzyl-3-O-methyl-α-D-rhamnopyranoside-(1→4)-2-O-acetyl-3-O-methyl-α-D-rhamnopyranoside-(1→4)-2-O-acetyl-3-O-methyl-α-D-rhamnopyranoside-(1→4)-2-O-acetyl-3-O-methyl-α-D-rhamnopyranoside-(1→4)-2-(tert-butoxycarbonyl)amino]ethyl 2-O-acetyl-3-O-methyl-α-D-rhamnopyranoside (S17)

Compounds S16 (878.9 mg, 0.91 mmol) and S8 (452.9 mg, 1.09 mmol) were coevapporated together with toluene (5×20 mL) and left to dry on high vacuum overnight. Anhydrous methylene chloride (50 mL) was added followed by 0.200 g of activated powdered 3 Å molecular sieves. The reaction was then cooled to −78° C. under an atmosphere of N₂ and N-iodosuccinimide (348.8 mg, 1.27 mmol) was added followed by triflic acid (0.15 mL, 1.70 mmol). The reaction was stirred for 3 h before reaching completion. The reaction was then filtered through a Buchner funnel, diluted with CH₂Cl₂ (50 mL), washed with 10% Na₂S₂O₃ (2×50 mL) and saturated NaHCO₃ (50 mL). The organic layer was then dried with Na₂SO₄, filtered, and purified by flash chromatography (eluent: EtOAc/Hexane, V/V) to yield compound S17 (693.0 mg, 0.55 mmol, 64%) as a clear oil. Rf=0.50 (EtOAc/Hexane, 3/2, V/V) [α]_{D}²⁵ 1.5 (c 0.27, CHCl₃) ¹H NMR (600 MHz, CDCl₃): δ 7.39-7.27 (m, 5H), 5.33 (s, 4H), 5.28 (s, 1H), 5.10 (s, 4H), 4.88 (d, 1H), 4.83 (br. s, 1H), 4.70 (s, 1H), 4.61 (d, 1H), 3.86-3.54 (m, 6H), 3.62-3.46 (m, 1 OH), 3.44 (s, 3H), 3.41-3.34 (m, 13H), 3.34-3.23 (m, 2H), 2.13 (s, 3H), 2.10 (s, 3H), 2.08 (s, 9H), 1.46 (s, 9H), 1.36-1.22 (m, 15H) ¹³C NMR (150 MHz, CDCl₃): δ 170.4, 170.3, 170.2, 170.1, 170.0, 155.9, 138.6, 128.5 (2C), 128.1 (2C), 127.9, 99.7, 99.6, 99.5, 99.4, 97.9, 80.1, 80.0, 79.9, 79.7, 78.8, 78.5, 78.3, 78.3, 77.4, 75.5, 68.7, 68.5, 68.0, 67.9, 67.8, 67.7, 67.3, 67.0, 57.6, 57.5, 57.4 (2C), 57.3, 40.4, 28.5, 21.2, 21.1, 18.4, 18.3, 18.2, 18.1, 17.9 ESI-MS: m/z Calcd for C₅₉H₉₁O₂₈NH [M+H]⁺, 1262.5800; found, 1262.5806.

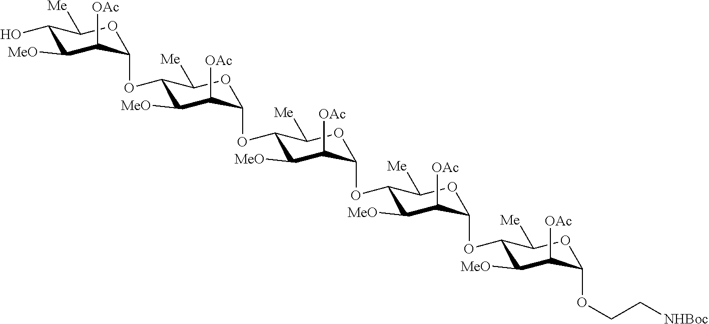

### 2-O-Acetyl-3-O-methyl-α-D-rhamnopyranoside-(1→4)-2-O-acetyl-3-O-methyl-α-D-rhamnopyranoside-(1→4)-2-O-acetyl-3-O-methyl-α-D-rhamnopyranoside-(1→4)-2-O-acetyl-3-O-methyl-α-D-rhamnopyranoside-(1→4)-2-(tert-butoxycarbonyl)amino]ethyl 2-O-acetyl-3-O-methyl-α-D-rhamnopyranoside (S18)

Palladium hydroxide on carbon (300.0 mg, 2.14 mmol) was added to MeOH (30 mL) and the solution was bubbled with H2 for 30 minutes. Next, compound S17 (623.3 mg, 0.49 mmol) was added and the solution was stirred under a continuous flow of H2 for 3 h at RT. The reaction was then filtered through a pad of celite and evaporated to afford S18 (537.0 mg, 0.46 mmol, 93%) as a clear oil. Rf=0.25 (EtOAc/Hexane, 3/2, V/V) [α]_{D}²⁵ 0.12 (c 0.033, CHCl₃) ¹H NMR (600 MHz, CD₃OD): δ 5.44-5.41 (m, 3H), 5.39 (s, 1H), 5.34 (dd, 1H), 5.11 (s, 1H), 5.09 (s, 2H), 5.07 (s, 1H), 4.76 (s, 1H), 3.92-3.83 (m, 3H), 3.83-3.77 (m, 2H), 3.77-3.73 (m, 1H), 3.70 (dd, 1H), 3.62-3.56 (m, 3H), 3.56-3.46 (m, 6H), 3.45 (3H), 3.45-3.38 (m, 15H), 3.34-3.24 (m, 2H), 2.14-2.12 (m, 12H), 2.11 (s, 3H), 1.52 (s, 9H), 1.36-1.32 (m, 12H), 1.31 (d, 3H) ¹³C NMR (150 MHz, CDCl₃): δ 171.7 (2C), 171.6 (3C), 158.4, 101.0, 100.8, 98.6, 81.4, 81.3, 81.2, 80.9, 80.3, 80.2, 81.1, 80.0, 79.6, 72.9, 70.7, 69.6, 69.3, 69.2, 69.1, 69.0, 68.9, 68.0, 67.5, 58.0, 57.6, 41.1, 29.0, 20.7, 18.6, 18.5 (3C), 17.9 ESI-MS: m/z Calcd for C₅₂H₈₅O₂₈NNa [M+Na]⁺, 1194.5150; found, 1194.5153.

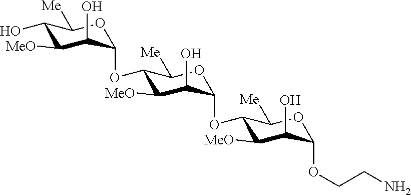

### 3-O-Methyl-α-D-rhamnopyranoside-(1→4)-3-O-methyl-α-D-rhamnopyranoside-(1→4)-2-(tert-butoxycarbonyl)amino]ethyl 3-O-methyl-α-D-rhamnopyranoside (S19)

Compound S14 (755.0 mg, 0.98 mmol) was dissolved in MeOH (30.0 mL) and sodium metal (15.0 mg, 0.65 mmol) was added. The reaction was stirred at RT for 16 h, neutralized with Dowex H⁺, filtered, and evaporated. The crude product was then dissolved in CH₂Cl₂ (100 mL) and trifluoroacetic acid (5.8 mL) and stirred at RT for 10 mins before being evaporated under reduced pressure to generate S19 (532.4 mg, 0.98 mmol, 100%) as a beige powder. [α]_{D}²⁵ 1.5 (c 0.52, CH₃OH) ¹H NMR (600 MHz, CD₃OD): δ 5.13 (s, 2H), 4.80 (s, 1H), 4.12 (s, 1H), 4.09 (s, 1H), 4.07 (s, 1H), 3.96-3.90 (m, 1H), 3.80-3.74 (m, 2H), 3.67-3.51 (m, 4H), 3.52-3.45 (m, 1H), 3.46 (s, 3H), 3.44 (s, 3H), 3.42 (s, 3H), 3.41-3.35 (m, 1H), 3.29-3.24 (m, 1H), 3.25-3.15 (m, 2H), 1.32 (d, 3H), 1.30 (d, 3H), 1.26 (d, 3H) ¹³C NMR (150 MHz, CD₃OD): δ 103.2, 103.1, 101.7, 83.1, 82.8, 82.0, 79.5, 79.3, 72.6, 70.5, 69.1, 68.7, 68.3, 68.0, 67.5, 64.6, 57.3, 56.7, 56.6, 40.5, 18.7, 18.6, 17.9 ESI-MS: m/z Calcd for C₂₃H₄₃O₁₃NH [M+H]⁺, 542.2807; found, 542.2804.

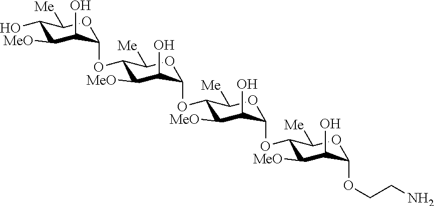

### 3-O-Methyl-α-D-rhamnopyranoside-(1→4)-3-O-methyl-α-D-rhamnopyranoside-(1→4)-3-O-methyl-α-D-rhamnopyranoside-(1→4)-2-(tert-butoxycarbonyl)amino]ethyl 3-O-methyl-α-D-rhamnopyranoside (S20)

Compound S16 (390 mg, 0.40 mmol) was dissolved in MeOH (30.0 mL) and sodium metal (10 mg, 0.44 mmol) was added. The reaction was stirred at RT for 16 h, neutralized with Dowex H⁺, filtered, and evaporated. The crude product was then dissolved in CH₂Cl₂ (50 mL) and trifluoroacetic acid (2.4 mL) and stirred at RT for 10 mins before being evaporated under reduced pressure to generate S20 (282.1 mg, 0.40 mmol, 100%) as a beige powder. [α]_{D}²⁵ 1.1 (c 0.13, CH₃OH) ¹H NMR (600 MHz, CD₃OD): δ 5.14 (m, 3H), 4.81 (s, 1H), 4.13 (s, 1H), 4.09 (s, 3H), 3.96-3.91 (m, 1H), 3.82-3.75 (m, 2H), 3.75-3.67 (m, 2H), 3.67-3.57 (m, 4H), 3.57-3.52 (m, 1H), 3.51-3.42 (m, 12H), 3.42-3.36 (m, 3H), 3.31-3.26 (m, 1H), 3.25-3.15 (m, 2H), 1.34 (d, 3H), 1.32-1.28 (m, 6H), 1.27 (d, 3H) ¹³C NMR (150 MHz, CD₃OD): δ 103.2, 103.1, 103.0, 101.7, 83.1, 83.0, 82.8, 79.4, 79.3, 79.2, 72.6, 70.5, 69.1, 68.7, 68.4, 68.4, 68.0, 67.5, 64.6, 57.3, 56.7, 56.6, 40.4, 18.7, 18.5 (2C), 17.9 ESI-MS: m/z Calcd for C₃₀H₅₅O₁₇NH [M+H]⁺, 702.3543; found, 702.3532.

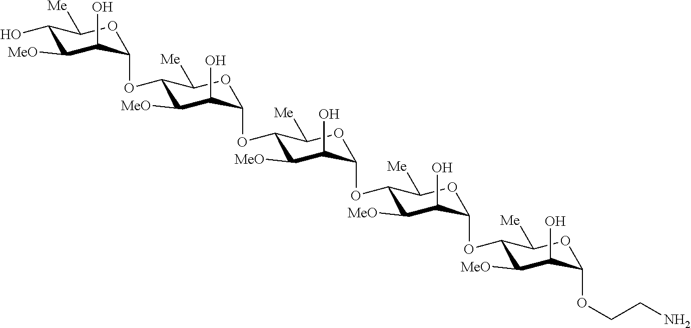

### 3-O-Methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranoside-(1→4)-2-(tert-butoxycarbonyl)amino]ethyl 3-O-methyl-α-D-rhamnopyranoside (S21)

Compound S18 (500 mg, 0.47 mmol) was dissolved in MeOH (30.0 mL) and sodium metal (10 mg, 0.44 mmol) was added. The reaction was stirred at RT for 16 h, neutralized with Dowex H⁺, filtered, and evaporated. The crude product was then dissolved in CH₂Cl₂ (50 mL) and trifluoroacetic acid (2.7 mL) and stirred at RT for 10 mins before being evaporated under reduced pressure to generate S21 (402 mg, 0.47 mmol, 100%) as a beige powder. [α]_{D}²⁵ 1.0 (c 0.26, CH₃OH) ¹H NMR (600 MHz, CD₃OD): δ 5.15 (s, 4H), 4.80 (s, 1H), 4.14-4.12 (m, 1H), 4.12-4.08 (m, 4H), 3.96-3.91 (m, 1H), 3.81-3.74 (m, 3H), 3.75-3.67 (m, 2H), 3.67-3.58 (5H), 3.57-3.52 (dd, 1H), 3.50-3.38 (m, 19H), 3.31-3.28 (dd, 1H), 3.26-3.15 (m, 2H), 1.34 (d, 3H), 1.32-1.28 (m, 9H), 1.27 (d, 3H) ¹³C NMR (150 MHz, CD₃OD): δ 103.2, 103.1, 103.0 (2C), 101.7, 83.1 (3C), 82.8, 82.0, 79.4, 79.3, 79.2 (2C), 72.6, 70.5, 69.1 (2C), 68.7, 68.4, 67.4 (2C), 67.5, 64.6, 57.3, 56.7, 56.6 (2C), 40.4, 18.7, 18.6 (3C), 17.9 ESI-MS: m/z Calcd for C₃₇H₆₇O₂₁NH [M+H]⁺, 862.4278; found, 862.4265.

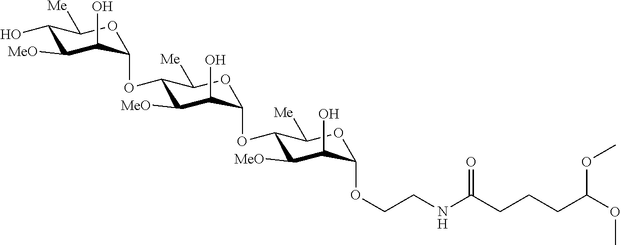

### 3-O-Methyl-α-D-rhamnopyranoside-(1→4)-3-O-methyl-α-D-rhamnopyranoside-(1→4)-2-(2,2-dimethoxybutylcarbonyl)amino]ethyl 3-O-methyl-α-D-rhamnopyranoside (S22)

2,5-Dioxopyrrolidin-1-yl 5,5-dimethoxypentanoate (239 mg, 0.92 mmol) was dissolved in anhydrous DMF (9.2 mL) and compound S19 (50 mg, 92 μmol) was added to this solution. The reaction was stirred for 16 h at RT and pushed to completion by the addition of 1 drop of Et₃N. The sample was evaporated under reduced pressure and coevaporated with toluene (5×10 mL). The crude sample was then suspended in H₂O (2 mL), extracted with CHCl₃ (5×1 mL). The aqueous layer was collected, evaporated, and purified by HPLC (C-18, H₂O/MeOH) to generate S22 (7.4 mg, 10.8 μmol, 12%) as a white powder. ¹H NMR (600 MHz, CD₃OD): δ 5.16-5.13 (m, 2H), 4.75 (d, 1H), 4.41 (t, 0.73H), 4.13-4.08 (m, 2H), 4.08-4.06 (m, 1H), 3.84-3.71 (m, 3.5H), 3.71-3.65 (m, 1H), 3.64-3.55 (m, 3H), 3.55-3.37 (m, 17H), 3.36-3.32 (m, 4H), 3.29 (dd, 1H), 2.26 (t, 1.6H), 1.73-1.60 (m, 3.3H), 1.33-1.25 (m, 9H) ¹³C NMR (150 MHz, CD₃OD): δ 176.2, 105.8, 103.2, 103.1, 101.3, 83.2, 83.1, 83.0, 82.0, 79.7, 79.2, 72.6, 70.5, 69.0, 68.3, 68.0, 67.6, 67.0, 57.3, 56.7, 53.5, 53.4, 40.2, 36.6, 33.1, 22.0, 18.7, 18.6, 17.8.

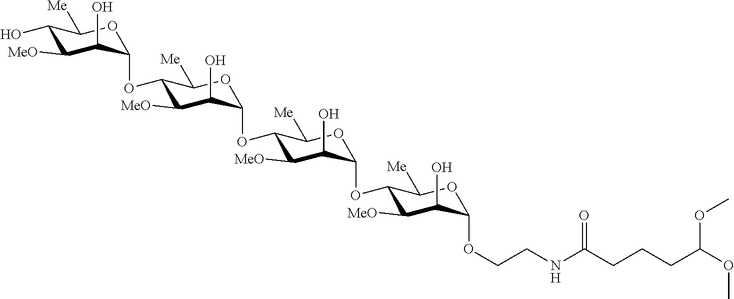

### 3-O-Methyl-α-D-rhamnopyranoside-(1→4)-3-O-methyl-α-D-rhamnopyranoside-(1→4)-3-O-methyl-α-D-rhamnopyranoside-(1→4)-2-(2,2-dimethoxybutylcarbonyl)amino]ethyl 3-O-methyl-α-D-rhamnopyranoside (S23)

2,5-Dioxopyrrolidin-1-yl 5,5-dimethoxypentanoate (185 mg, 0.71 mmol) was dissolved in anhydrous DMF (7.1 mL) and compound S20 (50.0 mg, 71.2 μmol) was added to this solution. The reaction was stirred for 16 h at RT and pushed to completion by the addition of 1 drop of Et₃N. The sample was evaporated under reduced pressure and coevaporated with toluene (5×10 mL). The crude sample was then suspended in H₂O (2 mL), extracted with CHCl₃ (5×1 mL). The aqueous layer was collected, evaporated, and purified by HPLC (C-18, eluent: H₂O/MeOH, V/V) to generate S23 (11.0 mg, 13.0 μmol, 18%) as a white powder. ¹H NMR (600 MHz, CD₃OD): δ 5.16-5.13 (m, 3H), 4.75 (d, 1H), 4.41 (t, 1H), 4.13-4.09 (m, 3H), 4.07 (dd, 1H), 3.82-3.71 (m, 4H), 3.71-3.65 (m, 1H), 3.64-3.56 (m, 3H), 3.54-3.47 (m, 6H), 3.47-3.36 (m, 14H), 3.35-3.32 (m, 6H), 3.31 (dd, 1H), 2.26 (t, 2H), 1.72-1.61 (m, 4H), 1.34-1.25 (m, 12H) ¹³C NMR (150 MHz, CD₃OD): δ 176.0, 105.8, 103.2, 103.1, 103.0, 101.3, 83.1, 83.0, 82.9, 82.0, 79.8, 79.4, 79.2, 72.6, 70.5, 69.1, 69.0, 68.4, 68.3, 68.0, 67.7, 67.0, 57.3, 56.7, 53.5, 53.4, 40.2, 36.6, 33.1, 22.0, 18.7, 18.6 (2C), 17.9.

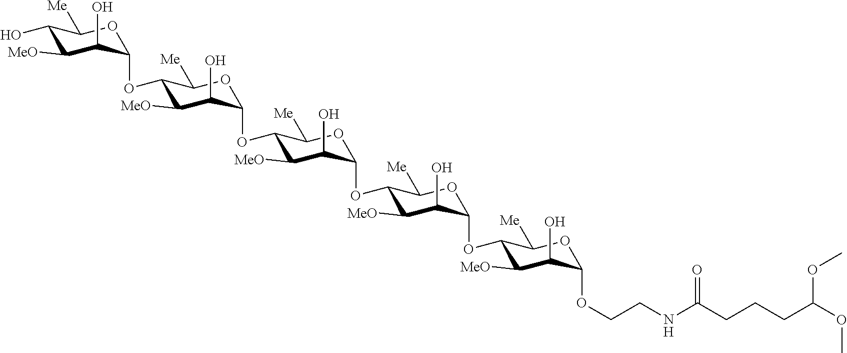

### 3-O-Methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranoside-(1→4)-2-(2,2-dimethoxybutylcarbonyl)amino]ethyl 3-O-methyl-α-D-rhamnopyranoside (S24)

2,5-Dioxopyrrolidin-1-yl 5,5-dimethoxypentanoate (150 mg, 0.58 mmol) was dissolved in anhydrous DMF (5.8 mL) and compound S21 (50 mg, 58.0 μmol) was added to this solution. The reaction was stirred for 16 h at RT and pushed to completion by the addition of 1 drop of Et₃N. The sample was evaporated under reduced pressure and coevaporated with toluene (5×10 mL). The crude sample was then suspended in H₂O (2 mL), extracted with CHCl₃ (5×1 mL). The aqueous layer was collected, evaporated, and purified by HPLC (C-18, eluent:H₂O/MeOH) to generate S24 (20.3 mg, 20.2 μmol, 35%) as a white powder. ¹H NMR (600 MHz, CD₃OD): δ 5.17-5.13 (m, 4H), 4.75 (d, 1H), 4.41 (t, 1H), 4.13-4.10 (m, 4H), 4.07 (dd, 1H), 3.82-3.71 (m, 5H), 3.71-3.65 (m, 1H), 3.65-3.56 (m, 4H), 3.52-3.38 (m, 22H), 3.36-3.33 (m, 7H), 3.31 (dd, 1H), 2.26 (t, 1H), 1.73-1.61 (m, 4H), 1.34-1.25 (m, 15H) ¹³C NMR (150 MHz, CD₃OD): δ 176.0, 105.8, 103.2, 103.1, 101.3, 83.1, 83.0, 82.0, 79.8, 79.4, 79.2, 72.7, 70.5, 69.0 (2C), 68.3, 68.0 (2C), 67.6, 67.1, 57.3, 56.7, 53.5, 53.4, 40.2, 36.6, 33.1, 22.0, 18.7, 18.6 (3C), 17.9.

### Example 6: Screening of New Oligosaccharide Linkers in Inhibition ELISA Vs mAb 1B1

The oligosaccharides with the linkers attached (from Example 5) are shown to effectively mimic the methyl rhamnan tip epitope. 1B1 mAb previously identified as specific for the methyl rhamnan tip at a constant concentration of 10 ug/ml in PBS-Tween was incubated at a ratio of 1:1 with dilutions of P. aeruginosa (Pa) PAO1 BAA-47 (wt) lipopolysaccharide (LPS) (positive control for inhibition) and N. meningitidis (Nm) galE lpt3 LPS (negative control for inhibition) and the synthetic oligosaccharides with linkers (S19-21). The final concentration of mAb 1B1 was 5 ug/ml. Pa and Nm LPS and the synthetic oligosaccharides were titrated starting at a concentration of 3 mg/ml (final 1.5 mg/ml) and diluted 2-fold, 12 times in PBS-Tween. This mixture was incubated together for 1h at room temp before adding to Pa wt LPS coated ELISA plates for 1h at room temp.

If enough LPS/oligosaccharide is present in the inhibition step to bind all of the available 1B1, then there will be no free 1B1 to bind the LPS on the ELISA plate and thus there will be no colour reaction in the ELISA. However, if the LPS/oligosaccharide either does not bind the antibody, or there is not enough LPS/oligosaccharide to block the 1B1 binding then the intensity of the colour generated in the ELISA will be similar to incubation with PBS alone.

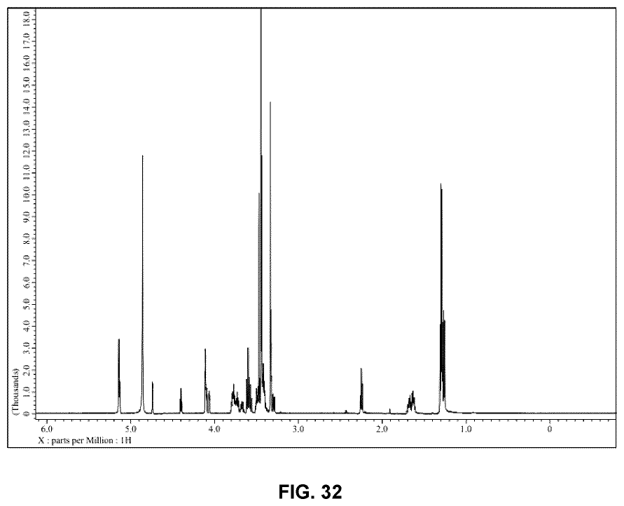

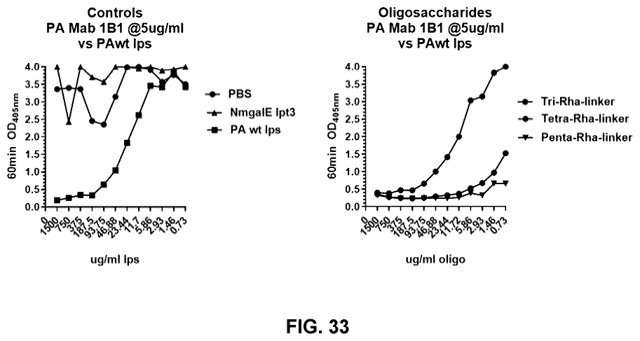

As shown in FIG. 33 Pa wt LPS binds and blocks 1B1, as no colour is generated in ELISA until the LPS is diluted to approximately 100 g/ml. The irrelevant Nm LPS does not block 1B1 from binding, nor does the PBS.

The oligosaccharides block 1B1 binding with the tetra- and penta-saccharide behaving similarly and only being titered out at approximately 5 g/ml, whereas the tri-saccharide also blocks binding but titers out earlier at approximately 100 g/ml.

These results therefore corroborate with the data from earlier prepared oligosaccharides (without linkers). The addition of the linker in these oligosaccharides does not alter the conformation of the oligosaccharide nor affect the oligosaccharide from binding and blocking mAb 1B1. Thus, the synthesised oligosaccharides with linkers effectively mimic the epitope recognised by mAb 1B1 on Pa.

### Example 7: Conjugation of Oligosaccharide Linkers to Activated CRM (for Immunisation) and Activated BSA (for Screening) to Prepare Glycoconjugates

Aminooxy activation of CRM& BSA: Initially the lysines of the protein (CRM or BSA) were activated by dissolving it at 10 mg/ml in 200 mM sodium phosphate buffer pH 7.4 and cooling to 4° C. Then an approximate 85× molar excess of bromoacetic acid N-hydroxysuccinimide ester dissolved in DMSO at 3 mg/ml was added and left 18 hrs at 4° C. The bromine activated protein was then desalted using an amicon ultra-10 30K MWCO spin column against water (three times) to an approximate volume of 500 μl. To this 500 μl of 200 mM sodium phosphate buffer pH 7.4 was added and cooled to 4° C. An approximate 75× molar excess of 3-(Aminooxy)-1-propanethiol Hydrochloride dissolved in 100 μl of in 200 mM sodium phosphate buffer pH 7.4 was added and left at room temperature for 2 hrs. The resulting aminooxy activated protein was then desalted using an amicon ultra-10 30K MWCO spin column against water (three times). Activation levels were determined by MALDI MS (Table 6)

Activation of linker to create aldehyde functionality: To convert the linkers on S22, S23 & S24 to the active aldehyde function, the oligosaccharides (S22-24) were dissolved at 3 mg/ml in 50% acetic acid and left at 37° C. for 7 hrs. Once cool the reaction mixture was then lyophilized.

Conjugation and characterisation of conjugates: 1 mg of the activated oligosaccharides (tri-, tetra- or penta-) dissolved in 50 μl of in 200 mM sodium phosphate buffer pH 6 was added to the activated protein at 0 hrs, 3 hrs, and 6 hrs. The amount of activated protein used was adjusted in each case to keep an approximate 8× molar excess of oligosaccharide per aminooxy of the protein. The reactions were left 18 hrs at room temperature. The product was then isolated using an amicon ultra-10 30K MWCO spin column against PBS (three times) and stored at 4° C.

3 mg of each of the activated oligosaccharides (tri-, tetra- and penta-) representing an approximate 8× molar ratio per aminooxy group on the activated CRM (2.1 mg/1.7 mg/1.5 mg CRM-oxy for the tri-/tetra-/penta- respectively) were conjugated. The degree of conjugation was determined by MALDI MS (Table 6)

TABLE 6
Summary of activation and oligosaccharide
loading of CRM & BSA conjugates

| | Amino-oxy activation | Conjugation |
|---|---|---|
| Conjugate | (# of amino-oxy/protein) | (# of oligos/protein) |
| CRM-tri | 15 | 10 |
| CRM-tetra- | 15 | 9 |
| CRM-penta | 15 | 8 |
| BSA-tri | 20 | 6 |
| BSA-tetra- | 20 | 5 |
| BSA-penta- | 20 | 6 |

### Example 8: Immunisation of Mice and Rabbits with Conjugates

Female BALB/c mice, 6- to 8-weeks-old, were immunised three times intraperitoneally. Each mouse received the same amount of oligosaccharide conjugate, as well as SIGMA adjuvant, and PBS buffer at each time point. The mice were primed on day 0 and received boosters on days 21 and 42, and blood samples were taken on day 0, day 35, and day 56.

Each mouse in group MRha3V received 3 ug of the trisaccharide conjugate resulting in 28 ug of CRM, along with 50% v/v SIGMA adjuvant, and PBS buffer totalling 100 ul, administered intraperitoneally. Each mouse in group MRha4V received 3 ug of the tetrasaccharide conjugate resulting in 25.5 ug of CRM, along with 50% v/v SIGMA adjuvant, and PBS buffer totalling 100 ul, administered intraperitoneally. And finally each mouse in group MRha5V received 3 ug of the pentasaccharide resulting in 23 ug of CRM, along with 50% v/v SIGMA adjuvant, and PBS buffer totalling 100 ul, administered intraperitoneally. Blood samples were obtained by submandibular vein collection method to yield approximately 100 ul of serum after blood separation.

### Example 9: Screening of Derived Mice Sera Vs. BSA-Conjugates and LPS

Individual sera from mice that had received a prime and two boost immunisation schedule were screened for their ability to recognise the BSA-oligosaccharide conjugates and Pa wt LPS in ELISA.

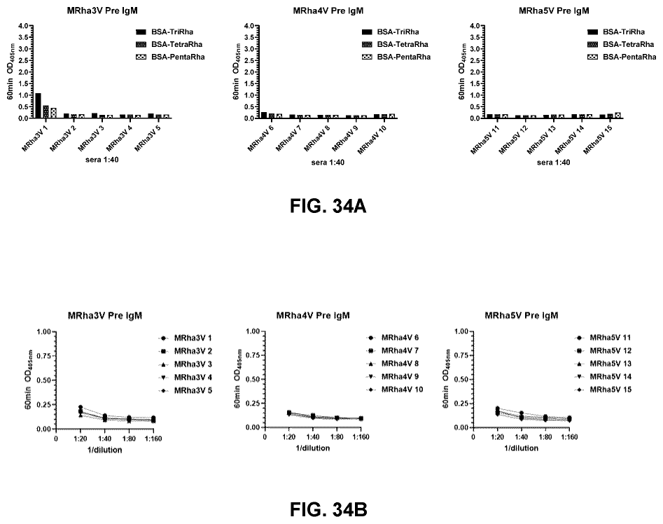

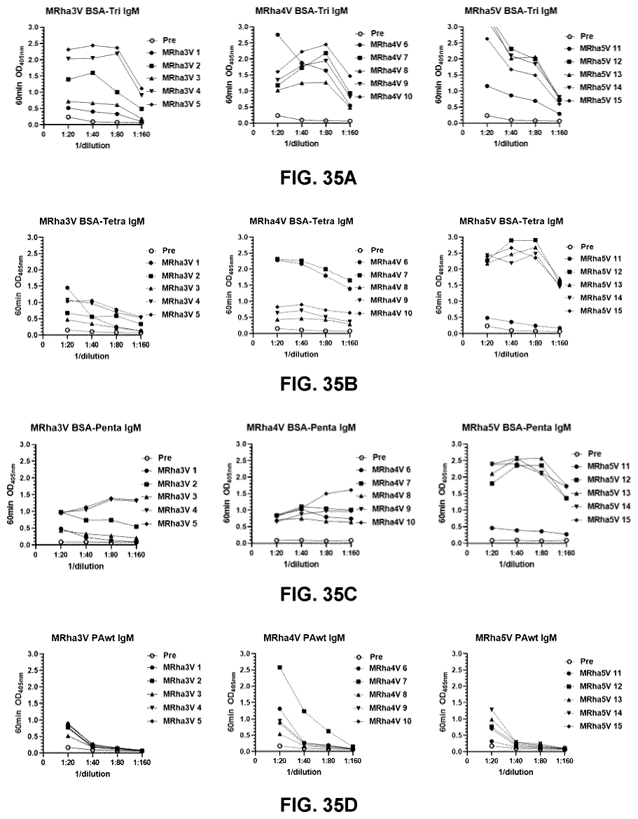

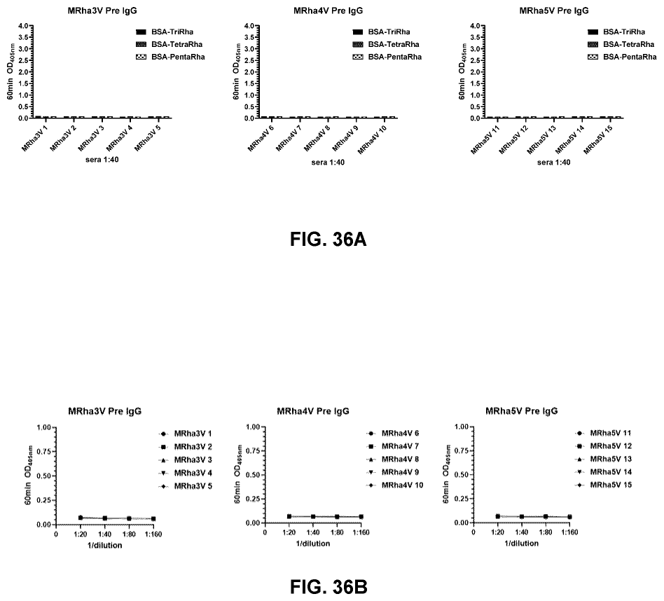

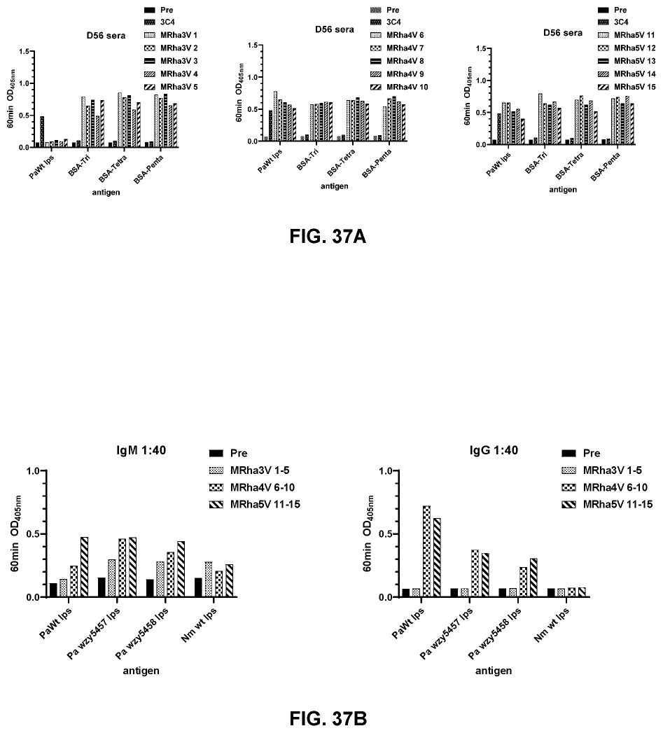

All mice produced a good IgM response to the conjugates as illustrated by their recognition of the BSA-oligosaccharide conjugates (FIG. 35A-C) in ELISA relative to the pre-immune sera (FIG. 34A) and a moderate response to the LPS (FIG. 35D) relative to the pre-immune sera (FIG. 34B). All mice produced a moderate IgG response to the conjugates as illustrated by their recognition of the BSA-oligosaccharide conjugates (FIG. 37A) in ELISA relative to the pre-immune sera (FIG. 36A). Similarly, all mice that received the tetra- and penta-saccharide conjugates produced a moderate IgG response to the conjugates as illustrated by their recognition of the LPS (FIG. 37B) in ELISA relative to the pre-immune sera (FIG. 36B). Since mice that received immunisations with the CRM-tetra- and pentasaccharide conjugates showed an improved IgG response to Pa wt LPS in ELISA relative to mice that received the tri-saccharide conjugate (FIG. 37B), it may be suggested from the mice data that the minimum length of oligosaccharide required to effectively mimic the natural antigen is a tetra-saccharide. Killed whole cells as detailed in Table 7 were screened in ELISA for recognition by the generated sera.

### Example 10: Screening of Derived Rabbit Sera Vs. BSA-Conjugates and LPS

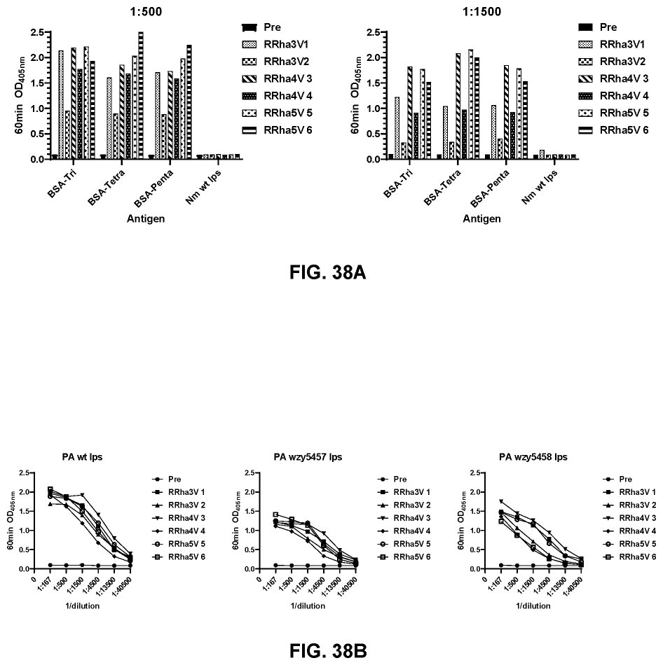

All rabbits produced a good immune response to the conjugates as illustrated by their recognition of the BSA-oligosaccharide conjugates in ELISA relative to the pre-immune sera (FIG. 38A). Similarly, all rabbits produced a good immune response to the conjugates as illustrated by their recognition of the LPS in ELISA relative to the pre-immune sera (FIG. 38B). All rabbits produced a strong response (end-point titers in the 1:10,000 range) that were capable of recognising several different LPS molecules.

### Example 11: Screening of Derived Mice and Rabbit Sera Vs. Killed Whole Cells

Whole cell ELISA was performed on a range of P. aeruginosa killed cells (Table 7) including a wild type strain, strains with mutations in genes thought to be related to the A-band methyl rhamnan and serotype strains most commonly encountered in a clinical setting.

TABLE 7
List of screened in whole cell ELISA.

| Species | Strain | NRCC # | Serotype | Details | Source |
|---|---|---|---|---|---|
| P. aeruginosa | PAO1 BAA-47 | 6678 | 5 | wild type | ATCC |
| | PAO1 | 6667 | — | PAO1 (wzy::Gm) | Lam lab |
| | PAO1 | 6668 | — | PAO1 (wzy::Gm)(Δpa5457) | Lam lab |
| | PAO1 | 6669 | — | PAO1 (wzy::Gm)(Δpa5458) | Lam lab |
| | PAO1 | 6670 | — | PAO1 (wzy::Gm)(Δpa5459) | Lam lab |
| | 5933 | 6954 | 1 | 33348 | ATCC |
| | 5934 | 6955 | 2 | 33349 | ATCC |
| | 5939 | 6956 | 6 | 33354 | ATCC |
| | 5943 | 6957 | 10 | 33357 | ATCC |
| | 5944 | 6958 | 11 | 33358 | ATCC |
| | 170003 | 6959 | 16 | 33363 | ATCC |
| | 5937 | 6960 | 5 | 33352 | ATCC |
| | 001S4-1 | 6944 | — | Clinical isolate | Sad lab |
| | 003S-20 | 6945 | — | Clinical isolate | Sad lab |
| | 003E-9 | 6946 | — | Clinical isolate | Sad lab |
| | 004S-3 | 6947 | — | Clinical isolate | Sad lab |
| | 004E-8 | 6948 | — | Clinical isolate | Sad lab |
| | 006S3-1 | 6949 | — | Clinical isolate | Sad lab |
| | 006S4-1 | 6950 | — | Clinical isolate | Sad lab |
| | 009S-7 | 6951 | — | Clinical isolate | Sad lab |
| | 014S-1 | 6952 | — | Clinical isolate | Sad lab |
| | 014E-31 | 6953 | — | Clinical isolate | Sad lab |
| M. catarrhalis | lgt2/lgt4 | 6541 | — | Negative control | Cox lab |
| N. meningitidis | 8047 lpt3 | 6263 | — | Negative control | Cox lab |

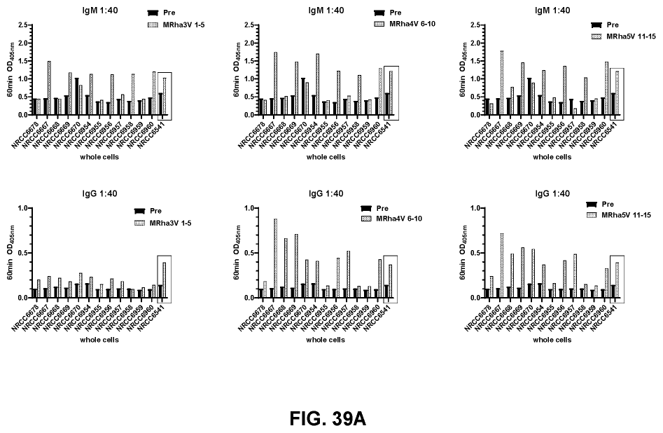

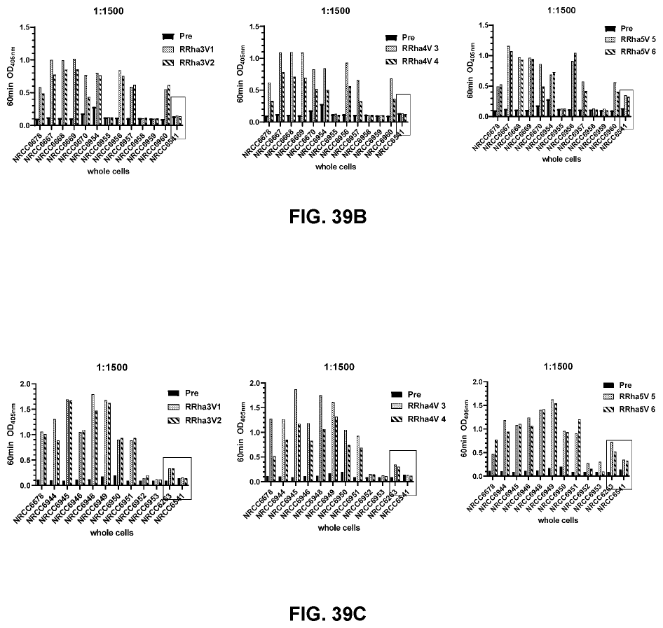

Post-immune mice sera pooled by oligosaccharide vaccine that they received recognised the cells as shown in FIG. 39A. Once again post-immune sera from the trisaccharide conjugates a weaker response when compared to the clear-cut response provoked by the tetra- and pentasaccharide conjugate illustrating that the methyl rhamnan tip epitope is visible in the context of whole cells and that the oligosaccharide conjugate was able to generate an immune response capable of recognising this epitope.

Post-immune individual rabbit sera recognised the cells as shown in FIG. 39B illustrating that the methyl rhamnan tip epitope is visible in the context of whole cells and that the oligosaccharide conjugates (tri-, tetra- and penta-) were able to generate an immune response capable of recognising this epitope. Furthermore, when the rabbit sera were screened (FIG. 39C) against a range of clinical isolates in our collection (Table 7) once again good cross-reactivity was observed against most of the strains, illustrating further the conservation of this epitope and the ability of oligosaccharidebased conjugates being capable of generating the required immune response.

Consistent with the earlier data with LPS and BSA conjugates, whole cell ELISA corroborated the requirement in mice for a tetrasaccharide as the minimum length of oligosaccharide necessary to facilitate an appropriate response. However, rabbit derived sera did not exhibit this same minimum length requirement, as all conjugates were able to elicit a similar cross-reactive response

Taken together these results indicate that CRM conjugates of at least the synthetic tetra- and pentasaccharides representative of the methyl rhamnan A-band tip epitope are capable of provoking a specific immune response that recognises the Pa wt LPS and whole cells representing the most commonly encountered serotypes in a clinical setting illustrating their potential as viable alternatives to the isolated antigens as vaccine immunogens.

The particular embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this specification are indicative of the level of skill those skilled in the art to which this invention pertains and are herein incorporated by reference to the same extent as if each individual publication, patent, or patent application was specifically and individually indicated to be incorporated by reference.

The invention being thus described, it will be obvious that the same may be varied in many ways. Such variations are not to be regarded as a departure from the spirit and scope of the invention, and all such modifications, as would be obvious to one skilled in the art, are intended to be included within the scope of the following claims.

### REFERENCES

The content of each of the following references is hereby incorporated by reference in its entirety.
1. Plackett, B., No money for new drugs, Nature 586 (2020) S50-S52.
2. Jansen, K. U., Anderson, A. S., The role of vaccines in fighting antimicrobial resistance, Hum. Vaccines Immunother. 14 (2018) 2142-2149.
3. Grimwood et al., Vaccination against respiratory Pseudomonas aeruginosa infection, Hum. Vaccines Immunother. 11 (2015) 14-20.
4. Adlbrecht et al., Efficacy, immunogenicity, and safety of IC43 recombinant Pseudomonas aeruginosa vaccine in mechanically ventilated intensive care patients—a randomized clinical trial, Crit. Care, 24 (2020) 74.
5. Merakou, C., Schaefers, M. M., Priebe, G. P., Progress toward the elusive Pseudomonas aeruginosa vaccine, Surg. Infect. 8 (2018) 757-768.
6. V. L. Campodónico et al., Efficacy of a conjugate vaccine containing polymannuronic acid and flagellin against experimental Pseudomonas aeruginosa lung infection in mice, Infect. Imun. 79 (2011) 3455-3464.
7. Hegerie et al., Development of a broad spectrum glycoconjugate vaccine to prevent wound and disseminated infections with Klebsiella pneuoniae and Pseudomonas aeruginosa, PLOS ONE (2018) https://doi.org/10.1371/journal.pone.0203143.
8. DiGiandomenico et al., Identification of broadly protective human antibodies to Pseudomonas aeruginosa exopolysaccharide Psl by phenotypic screening. J Exp Med 209 (2012) 1273-1287
9. DiGiandomenico et al., A multifunctional bispecific antibody protects against Pseudomonas aeruginosa. Sci Transl Med 6 (2014) 262ra155
10. Horn et al., Preclinical in vitro and in vivo characterization of the fully human monoclonal IgM antibody KBPA101 specific for Pseudomonas aeruginosa serotype IATS-O11. (2010) Antimicrob. Ag. Chemother. 54:2338-2344.
11. Loos et al., Pre-clinical and phase I safety data for anti-Pseudomonas aeruginosa human monoclonal antibody AR-105. (2019) Open Forum Infect. Dis. 6: 5307-5308.
12. AstraZeneca. 2019. AstraZeneca pipeline update. April 2020. https://www.astrazeneca.com/content/dam/az/PDF/2020/q12020/Q1_2020_results_clinical_trials_appendix.pdf
13. Patel et al., An engineered bispecific DNA encoded IgG antibody protects against Pseudomonas aeruginosa in a pneumonia challenge model. (2017) Nat. Commun. 8: 637-648.
14. Dasgupta et al., Characterization of lipopolysaccharide-deficient mutants of Pseudomonas aeruginosa derived from serotypes 03, 05, and 06. Infect. Immun (1994) 62:809-817
15. Byrd et al., Genetic and biochemical analyses of the Pseudomonas aeruginosa Psl exopolysaccharide reveal overlapping roles for polysaccharide synthesis enzymes in Psl and LPS production. Mol. Microbiol. (2009) 73:622-638
16. Sadovskaya et al., High-level antibiotic resistance in Pseudomonas aeruginosa biofilm: the ndvB gene is involved in the production of highly glycerol-phosphorylated beta-(1->3)-glucans, which bind aminoglycosides. Glycobiology 20 (2010) 895-904.
17. Knirel, Y. A. and Kocharova, N.A., Structure and properties of the common polysaccharide antigen of the bacterium Pseudomonas aeruginosa. Biochemistry (Moscow) 60 (1995) 1499-1507.
18. Yokota et al., Characterization of a polysaccharide component of lipopolysaccharide from Pseudomonas aeruginosa IID 1008 (ATCC 27584) as D-rhamnan. Eur. J. Biochem. 167 (1987) 203-209.
19. Vinogradov et al., Structures of lipopolysaccharides from Klebsiella pneumoniae. Eluicidation of the structure of the linkage region between core and polysaccharide O chain and identification of the residues at the non-reducing termini of the O chains. J Biol Chem. (2002) 277:25070-25081.
20. Wang et al., Biosynthesis of the Common Polysaccharide Antigen of Pseudomonas aeruginosa PAO1: Characterization and Role of GDP-D-Rhamnose: GlcNAc/GalNAc-Diphosphate-Lipid α1,3-D-Rhamnosyltransferase WbpZ. J. Bacteriol. (2015) 197: 2012-2019.
21. Arsenault et al., Structural studies on the polysaccharide portion of “A-band” lipopolysaccharide from a mutant (AK1401) of Pseudomonas aeruginosa strain PAO1. Can. J. Chem. 69 (1991) 1273-1280
22. Cox et al., Investigating the candidacy of a capsular polysaccharide-based glycoconjugate as a vaccine to combat Haemophilus influenzae type a disease: a vaccine solution for an unmet public health need. Vaccine (2017) 35:6129-36.
23. St. Michael et al., Investigating the candidacy of the serotype specific rhamnan polysaccharide based glycoconjugates to prevent disease caused by the dental pathogen Streptococcus mutans. Glycoconj. J. (2018) 35:53-64.
24. Cairns et al., Development and characterisation of mouse monoclonal antibodies specific for Clostridiodes (Clostridium) difficile lipoteichoic acid, utilising synthetic molecules to define a minimal epitope. ACS Chem. Biol. (2020) 15:1050-1058.
25. Makarenko et al., Immunological studies of an artificial antigen with specificity of a common polysaccharide antigen of Pseudomonas aeruginosa. FEMS Imm. Med. Microbiol. (1993) 7:251-256.
26. Lam et al., Occurrence of a common lipopolysaccharide antigen in standard and clinical strains of Pseudomonas aeruginosa. (1989) J. Clin. Microbiol. 27:962-967.
27. Richard et al., Antibody binding to the O-specific antigen of Pseudomonas aeruginosa O6 inhibits cell growth. Antimrob. Ag. Chemother. (2020) 64: e02168-19
28. Zunk, M. and Kiefel, M. J., An efficient synthesis of selectively functionalized d-rhamnose derivatives, Tetrahedron Lett., 52 (2011) 1296-1299.
29. Faure et al., Practical synthesis of valuable d-rhamnoside building blocks for oligosaccharide synthesis, Tetrahedron. Lett. 48 (2007) 2385-2388.
30. Seepersaud et al., Ceric ammonium nitrate/acetic anhydride: A tunable system for the O-acetylation and mononitration of diversely protected carbohydrates, Synth. Commun. 47 (2017) 853-871.
31. Blattner et al., Syntheses of oligomannosides in solution and on a soluble polymer support: a comparison, Carbohydr. Res. 341 (2006) 299-321.
32. Nagashima, N. and Ohno, M., An efficient O-monoalkylation of dimethyl 1-tartrate via O-stannylene acetal with alkyl halides in the presence of cesium fluoride, Chem. Lett., 16 (1987) 141-144.
33. Leontein, K. and Lonhgren, J., Determination of the absolute configuration of sugars by gas-liquid chromatography of their acetylated 2-octyl glycosides, in: J. N. BeMiller, R. L. Whistler, D. H. Shaw (Eds.), Methods in Carbohydrate Chemistry, Stockholm, John Wiley & Sons, Inc., 1993, pp. 87-89.
34. Wang, L. and Lowary, T. L., Synthesis of rhamnolipid derivatives containing ester isoteres, Org. Lett. 22 (2020) 9633-9637.
35. Domon, B. and Costello, C. E., A systematic nomenclature for carbohydrate fragments in FAB-MS/MS spectra of glycoconjugates, Glycoconjugate J, 5 (1988) 397-409.
36. Dass, C., Fundamentals of Contemporary Mass Spectrometry, Hoboken, New Jersey, John Wiley & Sons, Inc., 2007, pp. 17-26.
37. Crawford, C. J. and Oscarson, S., Convergent total synthesis of Cryptococcus neoformans serotype B capsule repeating motif, Carbohydr. Res. 497 (2020), https://doi.org/10.1016/j.carres.
38. Qin et al., Chemical synthesis of Pseudomonas aeruginosa O11 O-antigen trisaccharide based on neighboring electron-donating effect, J. Carbohydr. Chem. 39 (2020) 374-397.
39. X. Zhang, D. Wang, G. Jin, L. Wang, Z. Guo, G. Gu, Synthesis of a tetrasaccharide repeating unit of the exopolysaccharide from Burkholderia multivorans, J. Carb. Chem. 36 (2017) 189-204.
40. D. Wang, W. Zhuge, Z. Guo, G. Gu, Synthesis of a disaccharide repeating unit of the O-antigen from Burkholderia ambifaria and its oligomers, Carb. Res. 442 (2017) 41-51.
41. M. P. Jamshidi, C. Cairns, S. Chong, F. St. Micheal, E. V. Vinogradov, A. D. Cox, J. Sauvageau, Synthesis and Immunogenicity of a Methyl Rhamnan Pentasaccharide Conjugate from Pseudomonas aeruginosa A-Band Polysaccharide, ACS Infect. Dis. 8 (2022) 1347-1355.
42. Munneke S, Dangerfield E M, Stocker B L, Timmer M S M. The versatility of N-alkyl-methoxyamine bi-functional linkers for the preparation of glycoconjugates. Glycoconj J. 2017 October; 34(5):633-642. doi: 10.1007/s10719-017-9785-4. Epub 2017 Jul. 19. PMID: 28725972.
43. Bethell et al., 1979, J. Biol. Chem. 254:2572-4; Hearn et al., 1981, J. Chromatogr. 218:509-18.

What is claimed is:

1. An antigenic compound comprising the oligosaccharide moiety of Formula A:
α-Rha3OMe(-4α-Rha3OMe)ₙ- Formula A
wherein n is 1-5, preferably 2-4, and
wherein the 2-position in each Rha3OMe saccharide moiety is independently substituted with —OAc or —OH.

2. The antigenic compound of claim 1, having Formula A1:
α-Rha3OMe(-4α-Rha3OMe)ₙ-X Formula A1
wherein
n is 1-5, and
X is —H or -(4α-Man3OMe)ₘ-handle; and
m is 0, 1, or 2, preferably 0 or 1;
wherein the 2-position in each Rha3OMe saccharide moiety is independently substituted with —OAc or —OH.

3. The antigenic compound of claim 2, wherein the handle is 2-glyceraldehyde when m is 1 or 2, wherein the handle is —(CH2)_{z}NH₂ when m is 0, and wherein z is an integer selected from the group consisting of 1-5.

4. The compound of claim 1, selected from the group consisting of:
α-D-Rha3OMe-4-(α-D-Rha3OMe-4)₄-4-α-D-Man3OMe-2-glyceraldehyde-1d (OS2);
3-O-methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranose (pentasaccharide);
3-O-methyl-α-D-rhamnopyranoside-(1→4)-3-O-methyl-α-D-rhamnopyranoside-(1→4)-3-O-methyl-α-D-rhamnopyranoside-(1→4)-3-O-methyl-α-D-rhamnopyranose (tetrasaccharide)
3-O-methyl-α-D-rhamnopyranoside-(1→4)-3-O-methyl-α-D-rhamnopyranoside-(1→4)-3-O-methyl-α-D-rhamnopyranose (trisaccharide)

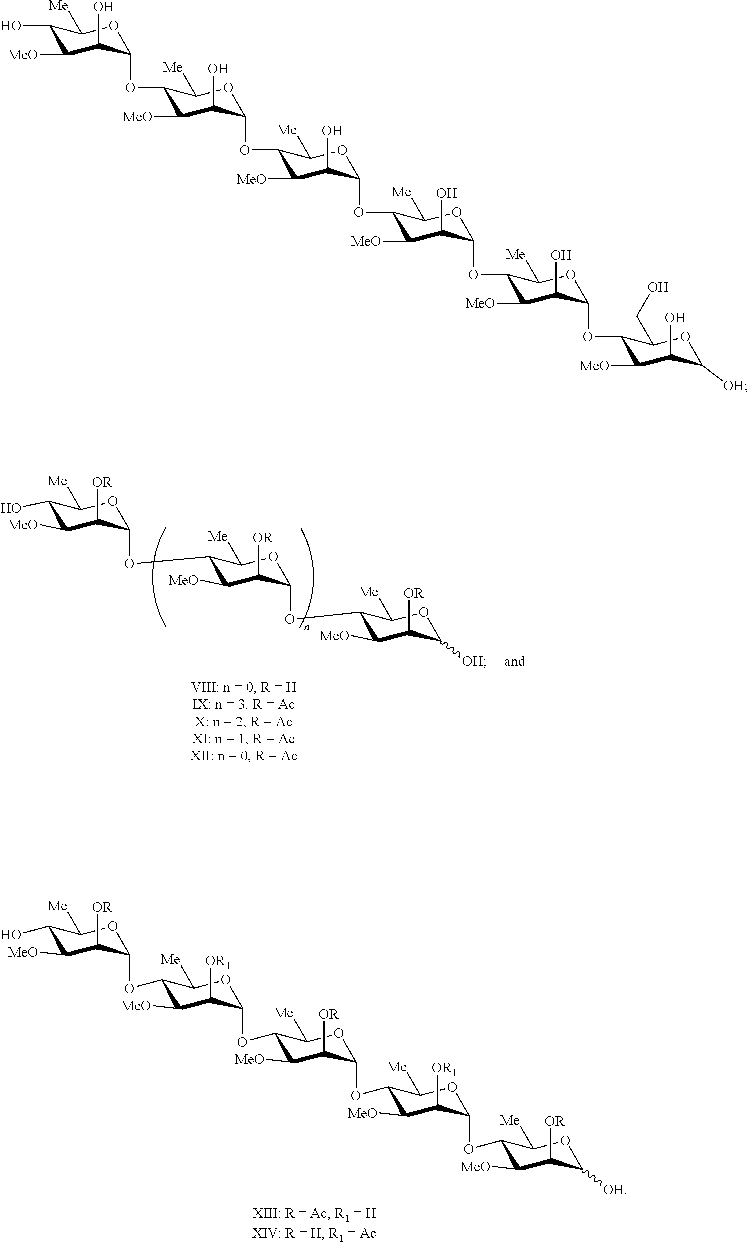

5. The antigenic compound of any one of claims 1 to 4, further comprising a linker for linkage to a carrier protein, and having Formula A2:
α-Rha3OMe(-4α-Rha3OMe)ₙ-X-Linker Formula A2
wherein
n is 1-5, and
X is -(4α-Man3OMe)ₘ-(handle)ₚ-;
m is 0, 1, or 2, preferably 0 or 1; and
p is 0 or 1
wherein the 2-position in each Rha3OMe saccharide moiety is independently substituted with —OAc or —OH.

6. A conjugate comprising the compound of any one of claims 1 to 5 conjugated to a carrier protein.

7. The conjugate of claim 6, wherein the carrier protein comprises CRM197, tetanus toxoid (TT), a Pseudomonas aeruginosa protein, human serum albumin (HSA), bovine serum albumin (BSA), diphtheria toxin fragment B (DTFB), DTFB C8, Diphtheria toxoid (DT), fragment C of TT, pertussis toxoid, cholera toxoid, E. coli LT, E. coli ST, or exotoxin A from Pseudomonas aeruginosa.

8. A pharmaceutical composition comprising
the compound of any one of claims 1 to 5 or the conjugate of claim 6 or 7; and
a pharmaceutically acceptable diluent, adjuvant, carrier, or excipient.

9. A vaccine comprising the compound of any one of claims 1 to 5, the conjugate of any one of claims 6 to 7, or the pharmaceutical composition of claim 8.

10. A method of raising an immune response in a subject, comprising administering to the subject: the compound of any one of claims 1 to 5, the conjugate of any one of claims 6 to 7, the pharmaceutical composition of claim 8, or the vaccine of claim 9.

11. A method of preventing a P. aeruginosa infection in a subject, the method comprising administering to the subject: the compound of any one of claims 1 to 5, the conjugate of any one of claims 6 to 7, the pharmaceutical composition of claim 8, or the vaccine of claim 9.

12. The compound of any one of claims 1 to 5, the conjugate of any one of claims 6 to 7, the pharmaceutical composition of claim 8, or the vaccine of claim 9 for use in preventing a P. aeruginosa infection.

13. An antibody, or an antigen binding fragment thereof, that selectively binds to
the compound of any one of claims 1 to 5,
the conjugate of any one of claims 6 to 7,
LPS of P. aeruginosa, and/or
a cell of P. aeruginosa.

14. An antibody, or an antigen binding fragment thereof, that selectively binds to an isolated oxidized A-band terminal epitope antigen (OS2) of formula:
α-D-Rha3OMe-4-(α-D-Rha3OMe-4)₄-4-α-D-Man3OMe-2-glyceraldehyde-1d (OS2).

15. The antibody or antigen binding fragment thereof of claim 13 or 14, wherein the antibody or antigen binding fragment thereof is a monoclonal antibody or antigen binding fragment thereof.

16. The antibody or antigen binding fragment thereof of any one of claims 13 to 15, which is a chimeric or humanized antibody.

17. The antibody or antigen binding fragment thereof of any one of claims 13 to 16, wherein the antibody or antigen binding fragment thereof comprises a heavy chain variable domain comprising a variable heavy chain CDR1, a variable heavy chain CDR2, and a variable heavy chain CDR3,
wherein the variable heavy chain CDR1 comprises an amino acid sequence selected from the group consisting of SEQ ID NO: 1, SEQ ID NO: 10, and SEQ ID NO: 19;
wherein the variable heavy chain CDR2 comprises an amino acid sequence selected from the group consisting of SEQ ID NO: 2, SEQ ID NO: 11, and SEQ ID NO: 20; and
wherein the variable heavy chain CDR3 comprises an amino acid sequence selected from the group consisting of SEQ ID NO: 3, SEQ ID NO: 12, and SEQ ID NO: 21.

18. The antibody or antigen binding fragment thereof of claim 17, further comprising a light chain variable domain comprising a variable light chain CDR1, a variable light chain CDR2, and a variable light chain CDR3,
wherein the variable light chain CDR1 comprises an amino acid sequence selected from the group consisting of SEQ ID NO: 6: SEQ ID NO: 15, and SEQ ID NO: 24;
wherein the variable light chain CDR2 comprises an amino acid sequence selected from the group consisting of GTS and RVS; and
wherein the variable light chain CDR3 comprises an amino acid sequence selected from the group consisting of SEQ ID NO: 7, SEQ ID NO: 16, and SEQ ID NO: 25.

19. The antibody or antigen binding fragment thereof of any one of claims 13 to 16, comprising a combination of a heavy chain variable domain (VH) and light chain variable domain (VL), wherein the combination is selected from the group consisting of:
a VH comprising the amino acid sequence of SEQ ID NO:4 and a VL comprising the amino acid sequence SEQ ID NO: 8;
a VH comprising the amino acid sequence of SEQ ID NO:13 and a VL comprising the amino acid sequence SEQ ID NO:17; and
a VH comprising the amino acid sequence of SEQ ID NO:22 and a VL comprising the amino acid sequence SEQ ID NO: 26.

20. The antibody or antigen binding fragment thereof of any one of claims 13 to 19, for use in the treatment of a P. aeruginosa infection.

21. The antibody or antigen binding fragment thereof of any one of claims 13 to 19, for use in the diagnosis of a P. aeruginosa infection.

22. A method for treating a P. aeruginosa infection, said method comprising administering the antibody or antigen binding fragment thereof of any one of claims 13 to 19 to a subject.

23. A method for the diagnosis of a P. aeruginosa bacterial infection in an animal, preferably human, comprising contacting a test sample with the antibody or antigen binding fragment thereof of any one of claims of any one of claims 13 to 19, and detecting specific binding thereto.

24. A synthetic process to produce the compound of Formula A1 of claim 2, wherein m is 0, said process comprising:
anomeric deprotection of a 3-O-methylated rhamnopyranoside to form a 3-O-methylated rhamnopyranose;
acetylating the 3-O-methylated rhamnopyranose to form an acetylated 3-O-methylated rhamnopyranoside;
partially deprotecting O-4 of the acetylated 3-O-methylated rhamnopyranoside to form a deprotected acetylated 3-O-methylated rhamnopyranoside;
coupling the O-4 deprotected acetylated 3-O-methylated rhamnopyranoside to form an acetylated 3-O-methylated oligosaccharide;
deprotecting O-4 of the acetylated 3-O-methylated oligosaccharide to form a O-4 deprotected acetylated 3-O-methylated oligosaccharide; and
deacetylating the partially deprotected acetylated 3-O-methylated oligosaccharide to form the compound of Formula A1.

25. A synthetic process to produce the compound of Formula A1 of claim 2, wherein X is a handle, said process comprising the following steps:
glycosylating an activated O-3 methylated rhamnopyranoside intermediate at 1-O with a handle comprising a protected amine, wherein the activated monorhamnopyranoside intermediate comprises a protecting group at glycosylation site 4-O, and forming a protected 1-O glycosidic intermediate;
removing the protecting group from 4-O and forming a deprotected 1-O glycosidic intermediate; and coupling the deprotected 1-O glycosidic intermediate to an activated O-3 methylated rhamnopyranoside intermediate, wherein the activated rhamnopyranoside intermediate comprises a protecting group at 4-O, and forming a protected methylated disaccharide, trisaccharide, tetrasaccharide or pentasaccharide; and removing all protecting groups from the protected disaccharide, trisaccharide, tetrasaccharide or pentasaccharide.
